(19) United States
(12) Patent Application Publication
(10) Pub. No.: US 2024/0216321 A1
(43) Pub. Date: Jul. 4, 2024

(54) FERMENTATIVELY-PRODUCED RETINOID CONTAINING COMPOSITIONS, AND THE METHDOS OF MAKING AND USING THE SAME

(71) Applicant: DSM IP Assets B.V., TE Heerlen (NL)

(72) Inventors: Adriana CARVALHO DE SOUZA, Columbia, MD (US); Peter Louis HOUSTON, Boston, MA (US); Valmik Kanubhai VYAS, Winchester, MA (US); Angela WILDERMANN, Bad Säckingen (DE)

(21) Appl. No.: 18/527,530

(22) Filed: Dec. 4, 2023

Related U.S. Application Data
(60) Provisional application No. 63/429,802, filed on Dec. 2, 2022.

(51) Int. Cl. A61K 31/23; A61K 31/01; A61K 31/045; A61K 31/07; A61K 31/11; A61K 31/231

(52) U.S. Cl. CPC A61K 31/23; A61K 31/01; A61K 31/045; A61K 31/07; A61K 31/11; A61K 31/231

(57) ABSTRACT

Compositions of matter suitable for use in food, feed, pharmaceutical, and beauty care products comprising a retinoid component and a fermentative residue thereof are disclosed herein. In an embodiment, the retinoid component is present in a ratio by weight relative to the fermentative residue of 4:1 or greater, wherein the retinoid contains cis- and trans-isomers, wherein the cis-isomers are present by weight relative to the entire mixture of isomers in an amount less than 3 wt. %. Preferably, the compositions are bio-based. Also described are biotechnological methods of producing the compositions elsewhere described, along with further methods for processing such compositions into more stabilized, more commercially acceptable forms. Finally, also disclosed are food, feed, pharmaceutical, and beauty care products incorporating the compositions herein described.

### CROSS REFERENCE TO RELATED APPLICATIONS

This application claims priority to U.S. provisional patent application No. 63/429,802, filed on 2 Dec. 2022, the entire contents of which is hereby incorporated by reference in its entirety as if fully set forth herein.

### REFERENCE TO SEQUENCE LISTING XML

Pursuant to the EFS-Web legal framework and 37 CFR §§ 1.821-825 (see MPEP § 2442.03(a)), a Sequence Listing in eXtensible Markup Language (XML) format (entitled “Sequence_Listing 34547-US-NP.xml” created on 4 Dec. 2023, and 79,488 bytes in size) is submitted concurrently with the instant application, and the entire contents of the Sequence Listing are incorporated herein by reference.

### TECHNICAL FIELD

The present invention relates to fermentatively-produced retinoid containing compositions, methods of producing the same, methods for formulating such fermentatively-produced retinoid containing compositions, and the formulations and (pre)products resulting therefrom.

### BACKGROUND

Retinoids are a class of chemical compounds that are vitamers of vitamin A or are chemically related thereto. Several retinoids have found use in a number of applications due to a variety of health benefits associated therewith. Specifically, retinoids have many important functions throughout the body including roles in vision, cell-proliferation regulation and differentiation, growth of bone tissue, immune function, and even activation of genes which may suppress tumors.

One particularly preferred retinoid, vitamin A, is a fat-soluble vitamin and an essential nutrient for humans. The vitamin encompasses several chemically related naturally occurring compounds or metabolites, i.e., vitamers, that all contain a β-ionone ring. It thus includes a group of organic compounds that includes retinol, retinal, retinoic acid, and retinyl acetate. Vitamin A has a multitude of uses, including for embryo development and growth, maintenance of the immune system, and vision.

Vitamin A occurs in a few principal forms in foods, such as retinol. It is also found in animal-sourced foods, either as retinol or bound to a fatty acid to become a retinyl ester, or the carotenoids alpha-carotene, β-carotene, gamma-carotene, and the xanthophyll beta-cryptoxanthin (all of which contain β-ionone rings) that function as provitamin A in herbivore and omnivore animals which possess the enzymes that cleave and convert provitamin carotenoids to retinal and then to retinol.

Vitamin A deficiency is common in developing countries. Deficiency can occur at any age but is most common in pre-school-age children and pregnant women, the latter due to a need to transfer retinol to the fetus. Vitamin A deficiency is estimated to affect approximately one-third of children under the age of five around the world, resulting in hundreds of thousands of cases of blindness and deaths from childhood diseases because of immune system failure.

Although vitamin A and vitamers thereof are produced biosynthetically in most animal species from the breakdown of β-carotene, industrial methods for its production rely on chemical synthesis. The first industrialized synthesis of retinol was achieved by the Hoffmann-La Roche in 1947. In the following decades, many other companies developed their own processes. β-ionone, synthesized from acetone, is the essential starting point for all industrial syntheses. Each process involves elongating the unsaturated carbon chain. Such chemical synthesis methods nonetheless tend to be energy-intensive and typically utilize raw materials from petroleum-based sources.

Biosynthesis alternatives are beginning to proliferate. Such methods which rely on fermentation tend to impart a significantly lower (i.e. on the order of 30-70%) carbon footprint than their chemical analogues, and further can incorporate starting materials of a bio-based—and therefore more circular—source.

Some methods utilize genetically engineered yeast species such as Saccharomyces cerevisiae to synthesize retinal and retinol, using xylose as a starting substrate. This was accomplished by having the yeast first synthesize β-carotene and then the cleaving enzyme β-carotene 15,15′-dioxygenase to yield retinal.

Yet other attempts to produce isoprenoids, and specifically retinoids, of biosynthetic origin (such as via oleaginous yeasts including, e.g. Yarrowia lipolytica) and using bio-based carbon sources during fermentation, are described by DSM IP Assets B.V. in, e.g. WO2022090548.

Regardless of the manufacturing method used, retinoids, and especially vitamin A and its vitamers, especially retinol, are extremely sensitive to oxidization and become self-heating in an oxygen-rich environment. In addition to causing degradation in the potency, bioavailability, and/or efficacy of the product with which they are associated, oxidization of retinoids can impart fire and other safety concerns. Such industrially-produced materials must therefore be prepared and transported at low temperatures and in oxygen-free atmospheres. When prepared as a dietary supplement or food additive, retinol is often stabilized as the ester derivatives retinyl acetate or retinyl palmitate, which tend to be slightly more oxidatively stable. Further, it is highly desirable to minimize the amount of impurities which by themselves or in conjunction with various retinoids would accelerate the degradation and/or oxidation process.

Despite the foregoing, a need continues to exist to configure and provide retinoid-containing compositions which are at least one of: contributing to greater circularity on account of a higher bio-based content, imparting a lower carbon footprint (especially when compared to existing industrially chemically-synthesized such compositions), and which can offer excellent oxidative stability, preferably comparable to or even superior to current industrially chemically-synthesized alternatives. Additionally or alternatively, a need exists for the provision of fermentatively-produced retinoid-containing compositions which are tuned for excellent and/or improved performance in their end-use application. One such example of this is configuration for excellent and/or improved antimicrobial efficacy, which can be important in a variety of cosmetic applications including but not limited to the prevention/treatment/lessening of symptoms of acne or malodor caused by sweating. The foregoing assures such retinoid-containing compositions may be commercially suitable for use in various products in a variety of food and beverage (hereinafter “food”), animal feed (hereinafter “feed”), pharmaceutical, and personal care applications.

### BRIEF SUMMARY

Inventors have herein discovered that one or more of the aforementioned problems can be solved when inventions according to various aspects and/or embodiments described herein are employed. Accordingly, described herein are several aspects and embodiments of the invention. A first aspect is a composition of matter for use in a food, feed, pharmaceutical, or beauty care product including (i) a retinoid component comprising a mixture of cis-isomers and trans-isomers; and (ii) a fermentative residue thereof; wherein, the retinoid component is present, relative to the fermentative residue thereof, in a ratio by weight of greater than 4:1; wherein the cis-isomers are present by weight, relative to the weight of the entire retinoid component, in an amount of less than 3 wt. %; optionally also wherein a bio-based carbon content of (i) and (ii) is greater than 50%, or greater than 60%, or greater than 70%, or greater than 90%. According to further embodiments of the first aspect, the composition of matter is configured to include one or more specific retinoids, including retinyl acetate, retinol, or retinyl palmitate, and/or the fermentative residue is configured to include one or more of fatty acid retinyl ester (FARE), retinal, retinol, farnesol, a fermentation carbon source, and/or β-carotene as the fermentative residue in varying amounts. In another embodiment of the first aspect, the composition is configured such that it possesses specified quantities of FARE and cis-isomers of the retinoid mixture. In yet further embodiments, the ratio of retinoid component (i) to fermentative residue (ii) is tuned to ranges other than that specified according to the first embodiment and/or the composition is configured to possess varying levels of bio-based carbon content. In still other embodiments, the composition is present in a crystalline form optionally also with specified average crystal lengths, or it may alternatively be present with a solvent as a “wet crystal” or even as an emulsion in an oil.

A second aspect of the invention is a method for fermentatively producing a retinoid-containing composition, the method comprising the steps of: (a) cultivating a microorganism under conditions that allow for production of a fermentation product; (b) isolating the fermentation product to yield an isolated retinoid-containing composition; wherein the isolated retinoid-containing composition comprises (i) a retinoid component comprising a mixture of cis-isomers and trans-isomers, and (ii) and a fermentative residue thereof; wherein, the retinoid component is present, relative to the fermentative residue thereof, in a ratio by weight of greater than 4:1, wherein the cis-isomers are present by weight, relative to the weight of the entire retinoid component, in an amount of less than 3 wt. %.

A third aspect of the invention is a method for preparing a food, feed, pharmaceutical, or personal care (pre)product comprising the steps of: (1) providing a composition of matter comprising, consisting of, or consisting essentially of (i) a retinoid component comprising a mixture of cis-isomers and trans-isomers; and (ii) a fermentative residue thereof; wherein, the retinoid component is present, relative to the fermentative residue thereof, in a ratio by weight of greater than 4:1; wherein the cis-isomers are present by weight, relative to the weight of the entire retinoid component, in an amount of less than 3 wt. %; optionally also wherein a bio-based carbon content of (i) and (ii) is greater than 50%, or greater than 60%, or greater than 70%, or greater than 90%; and (2) formulating the composition of matter to produce a formulation.

A fourth aspect of the invention is a beadlet or extrudate produced by the method of any of the embodiments of the third aspect of the invention and/or which incorporate the composition of matter according to any of the embodiments of the first aspect of the invention and/or made by any of the embodiments according to the second aspect of the invention.

A fifth aspect of the invention is a food, feed, pharmaceutical, or personal care product comprising any of the extrudates and/or beadlets according to the fourth aspect and/or any which were made according to any of the embodiments of the third aspect, and/or which include the composition of matter according to any of the embodiments of the first aspect and/or which were made by any of the methods of the second aspect.

### BRIEF DESCRIPTION OF THE DRAWINGS

FIG. 1 shows a micrograph image of a crystalline form of a fermentatively-produced retinoid-containing composition according to one or more aspects and/or embodiments of the current invention (Example 1) with select crystal length measurements indicated thereon; and

FIG. 2 shows a micrograph image of a crystalline form of a fermentatively-produced retinoid-containing composition according to one or more aspects and/or embodiments of the current invention (Example 2) with select crystal length measurements indicated thereon;

FIG. 3 shows a micrograph image of a crystalline form of a fermentatively-produced retinoid-containing composition according to one or more aspects and/or embodiments of the current invention (Example 3) with select crystal length measurements indicated thereon;

FIG. 4 shows a micrograph image of a crystalline form of a fermentatively-produced retinoid-containing composition according to one or more aspects and/or embodiments of the current invention (Example 4) with select crystal length measurements indicated thereon; and

FIG. 5 shows a micrograph image of a crystalline form of a chemically-synthesized retinoid-containing composition (Example 5) with select crystal length measurements indicated thereon.

### DETAILED DESCRIPTION

A first aspect of the current invention is a composition of matter for use in a food, feed, pharmaceutical, or beauty care product comprising, consisting of, or consisting essentially of
(i) a retinoid component comprising a mixture of cis-isomers and trans-isomers; and
(ii) a fermentative residue thereof;
wherein, the retinoid component is present, relative to the fermentative residue thereof, in a ratio by weight of greater than 4:1;
wherein the cis-isomers are present by weight, relative to the weight of the entire retinoid component, in an amount of less than 3 wt. %;
optionally also wherein a bio-based carbon content of (i) and (ii) is greater than 50%, or greater than 60%, or greater than 70%, or greater than 90%.

Compositions according to the first aspect of the invention contain both (i) a retinoid component and (ii) a fermentative residue thereof. As used herein, “retinoid component” signifies the specific retinoid compound that is the predominantly occurring retinoid which results after the fermentation process. The term “retinoid” as used herein preferably refers to compounds of formula (I) (without any indication of the stereochemistry):

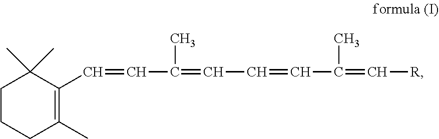

wherein
R is —CHO, —CH₂OH, —COOH, —CH(R¹)₂, —CH₂OR², —COOR³, —CONHR⁴, or —CO(NR⁴)₂,
R1 are independently lower alkoxy or R¹′ and R¹″ taken together are lower alkylenedioxy,
R² is alkanoyl or aroyl,
R³ is alkyl, aryl or aralkyl; and
R⁴, R^{4′} and R^{4″} are independently hydrogen, alkyl, aryl or aralkyl;
wherein the term “lower alkoxy” is an alkoxy group with 1 to 6 carbon atoms, such as, for example, methoxy, ethoxy or propoxy;
the term “lower alkylenedioxy” is such a group which likewise contains 1 to 6 carbon atoms, e.g., methylenedioxy or ethylenedioxy and wherein the alkyl or alkylene part can be straight-chain or branched depending on the number of carbon atoms;
the term “alkanoyl” refers to either straight-chain or branched alkanoyl groups with 1 to 18 carbon atoms, such as, for example, formyl, acetyl, propionyl, butyryl, stearoyl and palmitoyl;
the term “aroyl” refers to aromatic carboxylic acids with 7 and 11 carbon atoms, including benzoyl or naphthoyl, respectively;
the term “alkyl” is a straight-chain or branched alkyl group with 1 to 18 carbon atoms, e.g., methyl, ethyl, propyl, butyl, decyl, dodecyl, hexadecyl or octadecyl;
the term “aryl” as such or as part of “aralkyl” is phenyl or naphthyl; and
the term “aralkyl” embraces such groups with 1 to 4 carbon atoms in the aliphatic part, e.g., benzyl and phenylpropyl.

Particularly, compounds according to formula (I) as defined herein are selected from vitamin A and/or derivatives thereof including but not limited to one or more retinoids selected from retinol, retinal, retinoic acid, as well as derivatives such as esters, acetals and/or amides, particularly esters, more particularly retinyl acetate. Particularly suitable retinyl esters according to the present invention are retinyl acetate, retinyl propionate, retinyl linoleate, retinyl palmitate, retinyl retinoate and/or retinyl formyl aspartamate. Preferably, the compounds according to formula (I) are selected from retinol and/or retinyl esters, particularly retinol and/or retinyl acetate. In a preferred embodiment, the retinoid component consists of retinol. In another preferred embodiment, the retinoid component consists of retinyl acetate.

It is understood that the retinoid component may yield a retinoid of multiple isoforms, or isomers. In such instance, the retinoid component will include all such isomers. If a fermentation process yields multiple retinoid species, it is meant herein that only the most predominantly occurring retinoid (by weight %) is construed to be the retinoid component.

By extension, it will be understood that all other retinoid(s) produced will not, for purposes of the present invention, be construed as part of the “retinoid component”. Rather, such retinoids will form part of the “fermentative residue.” In addition to this, the fermentative residue will be construed herein also to include any other biologically-synthesized non-retinoid substance which is nonetheless produced by the microorganisms utilized during the fermentation process, such as precursor compounds like β-carotene, or derivative compounds such as a fatty acid retinyl ester (FARE) of a variety of types. Such substances can be referred to herein as “internal” fermentation residue, in the sense that they are produced by the microorganism itself, and not via introduction of a foreign substance to the process.

In addition to other retinoids not produced by the micro-organisms, however, the fermentative residue may additionally include any other substance or material that is the result of the process used to cultivate, ferment, and isolate the desired retinoid. This includes, therefore, any “external” impurities or byproducts which are introduced into the composition by virtue of the broader fermentation and extraction process, including, but not limited to: residual quantities of the carbon source used to feed the retinoid-producing microorganisms, any chemical additives used in the fermentation vessel or process, filtration media, washing fluid, solvents, biomass, or any other distillate. When construing the composition in the foregoing way, the fermentatively produced and isolated active retinoid—along with all impurities related thereto—can form a predefined composition of matter of an “active” ingredient, which can subsequently be incorporated via formulation process(s) into one or more commercially viable (pre)products.

In an embodiment, the fermentative residue of the composition comprises one or more of FARE, retinal, retinol, farnesol, a fermentation carbon source, and β-carotene.

It was well-known that fermentative residues were generally undesired “impurities” in the fermentation process. Specifically, it has been largely recognized that, with regards to the fermentative production of retinoids, particularly production of retinyl acetate, undesired “side-products” include but are not limited to formation of retinal, retinol, FAREs or dihydro-forms such as e.g. dihydro-retinol or dihydro-retinyl acetate, particularly FAREs, which should be reduced or abolished. Inventors have surprisingly discovered that certain fermentative residues, such as FAREs, may not be as undesired as previously thought, and may actually contribute to oxidative stability, if tuned in combination with one or more other factors. Specifically, inventors have surprisingly discovered that FARE content may tend to be far less detrimental to decreasing oxidative stability—the order of 10 times less important—than the corresponding quantity of cis-isomers of retinoid present in the retinoid component. Potentially more than this, even, inventors have observed that retinoid-containing compositions having small quantities of FARE are associated—or at least correlated—with increased oxidative stability relative to those which do not.

Therefore, in an embodiment the composition of the first aspect is configured so as to possess a so-called “weighted” select fermentative residue values (WSFRV), wherein WSFRV is determined according to the following formula:

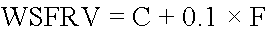

wherein
C=the percentage by weight of cis-isomers present relative to the total amount of retinoid component (i) and fermentative residue (ii), and
F=the percentage by weight of FARE present relative to the total amount of retinoid component (i) and fermentative residue (ii).

In certain embodiments, the WSFRV of the composition is configured to be between 0.1 and 2.5, or between 0.1 and 2.0, or between 0.1 and 1.0, or between 0.1 and 0.9, or between 0.4 and 2.5, or between 0.4 and 2.0, or between 0.4 and 1.0, or between 0.4 and 0.9, or between 0.6 and 2.5, or between 0.6 and 2.0, or between 0.6 and 1.0, or between 0.5 and 0.9.

As used herein, FARE is meant to include generally, without necessary limitation, all fatty acid retinyl esters as is understood generally in the art to which this invention applies. Three commonly-produced FAREs in fermentative production of various retinoids include retinyl acetate, retinyl palmitate, and retinyl oleate. As used herein, if the retinoid component is a retinoid other than retinyl acetate, retinyl palmitate, or retinyl oleate (such as retinol), then each of retinyl acetate, retinyl palmitate, and retinyl oleate will be included as FAREs in the fermentative residue. If, however, for example, retinyl acetate is produced as the retinoid component (i), then it will not also be counted as a FARE. In such case, retinyl palmitate and retinyl oleate will still be so-counted.

In other embodiments, the fermentative residue comprises one or more FAREs, wherein, relative to the total weight of retinoid component present, the one or more FAREs are present in an amount of less than 4 wt. %, or less than 3 wt. %, or less than 2 wt. %, or less than 1 wt. %. In still other alternative embodiments, the FARE is present, relative to the total retinoid component, in an amount between 0.05-4 wt. %, or between 0.1-4 wt. %, or between 0.15-4 wt. %, or between 0.2-4 wt. % of FARE, or between 0.05-3 wt. %, or between 0.1-3 wt. %, or between 0.15-3 wt. %, or between 0.2-3 wt. % of FARE; or between 0.05-2 wt. %, or between 0.1-2 wt. %, or between 0.15-2 wt. %, or between 0.2-2 wt. % of FARE; or between 0.05-1 wt. %, or between 0.1-1 wt. %, or between 0.15-1 wt. %, or between 0.2-1 wt. % of FARE.

In other embodiments, the composition is configured such that, instead of a WSFRV as specified above, it possesses both a mixture of cis-isomers and trans-isomers of the retinoid component, preferably wherein the retinoid component consists of retinyl acetate or retinol, and wherein the cis-isomers are present by weight, relative to the weight of the entire mixture of cis- and trans-isomers, in an amount of less than 1 wt. %;
and/or
wherein, relative to the total weight of the retinoid component, FARE is present in an amount of less than 3.5 wt. %, or less than 1.5 wt. %.

In various embodiments, the retinoid component comprises a mixture of cis- and trans-isomers, wherein the cis-isomers are present by weight, relative to the weight of the entire mixture, between
0.1-3 wt. %, or between 0.2-3 wt. %, or between 0.4-3 wt. %, or between 0.5-3 wt. %; or 0.1-2.5 wt. %, or between 0.2-2.5 wt. %, or between 0.4-2.5 wt. %, or between 0.5-2.5 wt. %; or
0.1-2 wt. %, or between 0.2-2 wt. %, or between 0.4-2 wt. %, or between 0.5-2 wt. %; or 0.1-1.5 wt. %, or between 0.2-1.5 wt. %, or between 0.4-1.5 wt. %, or between 0.5-1.5 wt. %; or
0.1-1 wt. %, or between 0.2-1 wt. %, or between 0.4-1 wt. %, or between 0.5-1 wt. %; or
0.1-0.9 wt. %, or between 0.2-0.9 wt. %, or between 0.4-0.9 wt. %, or between 0.5-0.9 wt. %.

With regards to the retinoid component (i), as stated it is understood that both cis- and trans-isomers of the specific retinoid will be present. As used herein, the term “trans-retinyl acetate”, “trans-retinol”, “trans-retinal” or the like are known to the skilled person and in line with the IUPAC-IUB nomenclature and mean that all double bonds in such retinyl acetate, retinol, retinal compound, including the compound according to formula (I), are in trans configuration. The term “trans-retinyl ester”, particularly “trans-retinyl acetate”, and “all-trans-retinyl ester”, particularly “all-trans-retinyl acetate”, are used interchangeably herein.

Thus, if any one of several double bonds is not in the trans configuration but in the cis configuration, it will be understood herein that all such isomers are considered herein to be cis-isomers (whether, e.g. cis-retinyl acetate or cis-retinol or cis-retinal, depending upon the retinoid component (i) desired) and are therefore included in the determination of “cis-isomer %” or “cis-isomer content.” Conversely, only those isomer(s) which possess all double bounds in the trans configuration will be construed herein to be trans-isomers.

By way of example, retinoid isomers in cis-configuration that are covered by the present invention include, but are not limited, to 9-cis-retinol, 11-cis retinol, 13-cis retinol, 9,13-di-cis-retinol, 11,13-di-cis retinol, 13-cis-3,4-didehydro retinol, 9-cis-3,4-didehydro retinol, 9,13-di-cis-3,4-didehydro retinol, 13-cis retinal, 11-cis-retinal, 11,13-di-cis retinal, 9,13-di-cis-retinal, 9-cis retinal, 13-cis-3,4-didehydro retinal, 11,13-di-cis-3,4-didehydro retinal, as well as the respective cis-forms of retinyl acetate (for review, see e.g. Table 1 in Gundersen and Blomhoff, J. Chromatogr. A 935, 13-43, 2001).

Inventors have discovered that retinoid-containing compositions with retinoid components configured to possess certain contents of cis- and trans-isomers, respectively, can impart certain advantages to the composition or (pre)product with which they are associated. As a non-limiting potential advantage, such compositions can be used as antimicrobial agents with cis/trans ratios as defined herein. Specifically, with retinyl acetate as retinoid component, the composition has been observed to be particularly suitable as anti-microbial agent against Cutibacterium acnes, Corynebacterium xerosis and Malassezia, preferably M. furfur, while retinol is particularly suitable as antimicrobial agent against Cutibacterium acnes.

The cis/trans ratio as given herein refers to the wt.-% ratio of the respective all trans isomers to the sum of all cis-isomers of retinoids/mix of retinoids according to formula (I), as determined by HPLC, assuming the same response factor for all isomers. In preferred embodiments of the first aspect of the present invention, the cis/trans ratio of compounds according to formula (I) is less than 0.03, or less than 0.02, or less than 0.01, or a cis/trans ratio of 0.0099, 0.0095, 0.009, 0.0085, 0.008, 0.0075, 0.007, 0.0065, 0.006, 0.0055, 0.005, 0.0049, 0.0045, 0.0043, 0.004, 0.0035, 0.003, 0.0028, 0.0025, 0.0022, 0.002, 0.0018, 0.0015, 0.001, 0.0005 or less, such as 0.0001 or less, more preferably ranges of 0.0099 to 0.0001, 0.008 to 0.001, 0.007 to 0.002, 0.0099 to 0.001, 0.005 to 0.001, 0.005 to 0.0001, 0.005 to 0.0005, 0.006 to 0.001, 0.009 to 0.0001, 0.008 to 0.0001, 0.006 to 0.0001, 0.0055 to 0.0001, 0.005 to 0.002, 0.007 to 0.0001, 0.0045 to 0.0001, 0.004 to 0.0001, 0.0035 to 0.0001, 0.003 to 0.0001, 0.005 to 0.0025, 0.004 to 0.0005, 0.006 to 0.0005, 0.006 to 0.0008, 0.006 to 0.0015, 0.006 to 0.002, 0.006 to 0.003, 0.006 to 0.0004, 0.006 to 0.0025, most preferably a cis/trans ratio of 0.0049 to 0.0025.

In one preferred embodiment, the retinoid according to formula (I) is retinyl acetate, particularly with a cis/trans ratio of less than 0.03, or less than 0.02, or less than 0.01, preferably 0.0099, 0.0095, 0.009, 0.0085, 0.008, 0.0075, 0.007, 0.0065, 0.006, 0.0055, 0.005, 0.0049, 0.0045, 0.0043, 0.004, 0.0035, 0.003, 0.0028, 0.0025, 0.0022, 0.002, 0.0018, 0.0015, 0.001, 0.0005 or less, such as 0.0001 or less, more preferably ranges of 0.0099 to 0.0001, 0.008 to 0.001, 0.007 to 0.002, 0.009 to 0.0001, 0.008 to 0.0001, 0.005 to 0.001, 0.005 to 0.0001, 0.005 to 0.0005, 0.006 to 0.001, 0.006 to 0.0001, 0.0055 to 0.0001, 0.007 to 0.0001, 0.0045 to 0.0001, 0.005 to 0.002, 0.004 to 0.0001, 0.0035 to 0.0001, 0.003 to 0.0001, 0.005 to 0.0025, 0.006 to 0.0005, 0.006 to 0.0008, 0.006 to 0.0015, 0.006 to 0.002, 0.006 to 0.003, 0.006 to 0.0004, 0.006 to 0.0025, 0.004 to 0.0005, most preferably a cis/trans ratio of 0.0049 to 0.0025.

In alternative embodiments, the cis/trans ratio can be stated differently, such as by total percentage of cis-isomer content. Accordingly, in various embodiments, the retinoid component comprises a mixture of cis- and trans-isomers, wherein the cis-isomers are present by weight, relative to the weight of the entire mixture, between
0.1-3 wt. %, or between 0.2-3 wt. %, or between 0.4-3 wt. %, or between 0.5-3 wt. %; or
0.1-2.5 wt. %, or between 0.2-2.5 wt. %, or between 0.4-2.5 wt. %, or between 0.5-2.5 wt. %; or
0.1-2 wt. %, or between 0.2-2 wt. %, or between 0.4-2 wt. %, or between 0.5-2 wt. %; or
0.1-1.5 wt. %, or between 0.2-1.5 wt. %, or between 0.4-1.5 wt. %, or between 0.5-1.5 wt. %; or
0.1-1 wt. %, or between 0.2-1 wt. %, or between 0.4-1 wt. %, or between 0.5-1 wt. %; or
0.1-0.9 wt. %, or between 0.2-0.9 wt. %, or between 0.4-0.9 wt. %, or between 0.5-0.9 wt. %.

Compositions comprising retinyl acetate with a cis/trans ratio as above may be useful for prevention, treatment or lessening the symptoms of acne, itching skin, Pityriasis versicolor, dandruff formation, seborrheic dermatitis atopic dermatitis, psoriasis and/or malodor caused by sweat, particularly useful as antimicrobial agents against Cutibacterium acnes, Corynebacterium xerosis and/or Malassezia, preferably M. furfur.

In one preferred embodiment, the retinoid according to formula (I) is retinol, particularly with a cis/trans ratio of less than 0.03, or less than 0.02, or less than 0.01, preferably a cis/trans ratio of 0.0099, 0.0095, 0.009, 0.0085, 0.008, 0.0075, 0.007, 0.0065, 0.006, 0.0055, 0.005, 0.0049, 0.0045, 0.0043, 0.004, 0.0035, 0.003, 0.0028, 0.0025, 0.0022, 0.002, 0.0018, 0.0015, 0.001, 0.0005 or less, such as 0.0001 or less, more preferably ranges of 0.0099 to 0.0001, 0.008 to 0.001, 0.007 to 0.002, 0.0099 to 0.001, 0.005 to 0.001, 0.005 to 0.0001, 0.005 to 0.0005, 0.006 to 0.001, 0.009 to 0.0001, 0.008 to 0.0001, 0.006 to 0.0001, 0.0055 to 0.0001, 0.005 to 0.002, 0.007 to 0.0001, 0.0045 to 0.0001, 0.004 to 0.0001, 0.0035 to 0.0001, 0.003 to 0.0001, 0.005 to 0.0025, 0.004 to 0.0005, 0.006 to 0.0005, 0.006 to 0.0008, 0.006 to 0.0015, 0.006 to 0.002, 0.006 to 0.003, 0.006 to 0.0004, 0.006 to 0.0025, most preferably a cis/trans ratio of 0.003. Compositions comprising retinol with a cis/trans ratio as above may be useful for prevention, treatment and/or lessening the symptoms of acne, particularly useful as antimicrobial agents against Cutibacterium acnes.

The mixture of cis and trans isomers of retinoids according to formula (I), particularly retinyl esters, preferably retinyl acetate, and/or retinol according to the present invention can either be prepared by admixing the respective all trans isomers with one or more cis isomers obtained by chemical or biological processes. Methods to prepare such all trans and/or cis isomers are well known to a person skilled in the art. Alternatively, the mixture can be prepared in said isomer ratio by adjusting the processes accordingly.

Advantageously, in preferred embodiments of the first aspect of the present invention, the retinoids, particularly retinyl esters such as e.g. retinyl acetate, and/or retinol are biologically produced through a fermentation process.

In a particular embodiment, the mixture and/or composition comprising said mixture of cis and trans isomers of retinoids as defined herein, particularly retinyl esters, preferably retinyl acetate, and/or retinol are produced in a fermentation process using suitable retinol producing host cells, such as e.g. bacterial or fungal cells, (see e.g. Sun et al, ACS Synth. Biol. 2019 Sep. 20; 8(9):2131-2140; Jang et al., Microbial Cell Factories 2011, 10:59), wherein the cells are expressing the respective enzymes, such as e.g. trans-selective enzymes, i.e. beta-carotene oxidase (BCOs) involved in biosynthesis of retinol from conversion of beta-carotene into retinal, that might be further enzymatically converted into retinol and/or retinyl esters, particularly retinyl acetate. The fermentation is fed ethanol, corn sugar or corn oil all derived from agricultural production. The fermentation products comprising retinyl esters, particularly retinyl acetate, might be extracted in an aliphatic phase and subsequently purified to crystalline forms.

Advantageously, trans retinyl esters, particularly trans retinyl acetate, and/or trans-retinol produced by fermentation can be treated with heat to form cis retinyl esters, particularly cis retinyl acetate, and/or cis-retinol to achieve the proper levels mentioned in all the embodiment of the present invention embodiment (see e.g. McBee et al., JBC, Vol. 276, No. 51, pp. 48483-48493, 2001).

The term “biologically produced” or “fermentatively produced” as used herein means that the retinoids, particularly retinyl esters, particularly retinyl acetate, and/or retinol is produced by the help of biotechnological process, such as a fermentation process including cultivation of a suitable (carotenoid and/or retinoid producing) host cell expressing the respective enzymes involved in conversion of a suitable carbon source into retinyl esters, particularly retinyl acetate, and/or retinol as defined herein, wherein the host cell might be selected from bacteria, fungi, particularly yeast, plant or algae. “Bio-produced”, “biologically-derived” and “biologically produced” are used synonymously herein. Accordingly, said biologically produced retinoids according to formula (I) is composed of carbon from atmospheric carbon dioxide (also referred to as carbon of atmospheric origin) converted to sugars and starches by green plants. It also includes the use of isolated and/or immobilized enzymes in a process for generation of the retinoid mixtures as defined herein, such as specific enzymes capable of selectively catalyzing the formation of the specific trans/cis ratio of retinoids, particularly retinyl esters, preferably retinyl acetate, and/or retinol in a mixture as defined herein.

In an embodiment, a “bio-based” compound has a C-14/C-12 isotope ratio in the range of from 1:0 to greater than 0:1, as in contrast to a fossil-based compound with a C-14/C-12 isotope ratio of 0:1. Bio-based content of a compound can be measured by known radiocarbon and isotope ratio mass spectrometry analysis or accelerator mass spectrometry, such as e.g. the ASTM test method D6866-05, wherein the C-14/C-12 isotope ratio in a sample is measured and compared to a standard 100% biobased material to give percent biobased content of the sample.

“Carbon of atmospheric origin” as used herein refers to carbon atoms from carbon dioxide molecules that have recently, in the last few decades, been free in the earth's atmosphere. Such carbons in mass are identifiable by the presence of particular radioisotopes as described herein. “Green carbon”, “atmospheric carbon”, “environmentally friendly carbon”, “life-cycle carbon”, “non-fossil fuel-based carbon”, “non-petroleum based carbon”, “carbon of atmospheric origin”, and “biobased carbon” are used interchangeably herein.

According to certain preferred embodiments of the present invention, advantageously the retinoids, particularly retinol and/or retinyl esters, preferably retinyl acetate, are produced by merely organic, renewable, bio-based feedstock, particularly fermentatively produced, as such retinol and/or retinyl esters, preferably retinyl acetate, has an anthropogenic CO₂ emission profile of zero upon biodegradation because all of the CO₂ molecules released during degradation from such “fermentatively-derived” or “fermentatively-produced” retinoids have an atmospheric origin. Thus, the net release of CO₂ to the atmosphere is zero. In a preferred embodiment, therefore, the fermentation carbon source comprises, consists of, or consists essentially of a biogenic carbon source.

In various embodiments of the first aspect, the the bio-based carbon content of the composition of matter is greater than 60%, or greater than 70%, or greater than 80%, or greater than 90%, or greater than 99%, or even approximately 100%; in yet other embodiments, the bio-based carbon content of the retinoid component (i) and the fermentative residue thereof (ii) is greater than 60%, or greater than 70%, or greater than 80%, or greater than 90%, or greater than 99%, or even approximately 100%. The bio-based content may be determined according to any suitable means, such as via ASTM D6866-20 or by way of appropriate ¹⁴C isotope characterization, such as the method described elsewhere herein, infra.

As stated, the fermentative residue can contain a variety of substances, such as one or more of FARE, beta-carotene, retinol, retinal, and/or a fermentation carbon source. In other embodiments, particularly where the retinoid component is retinyl acetate, the fermentative residue may comprise each of FARE, beta-carotene, retinol, retinal, and a fermentation carbon source.

A variety of retinol isotopes may be included as the retinol, such as, for example E-retinol. Similarly, a variety of retinal isotopes may be used as well, such as 9Z-retinal. Inventors have surprisingly observed that compositions containing 9Z-retinal can exhibit improved oxidative stability when compared with those not containing such fermentative residue, although the oxidative stability interestingly seems to be inversely correlated to the amount of 9Z-retinal present in the retinoid-containing composition.

Therefore, in various embodiments of the first aspect, the fermentative residue comprises 9Z-retinal, but wherein, relative to the total weight of retinoid component present, 9Z-retinal is present in an amount of less than 10 wt. %, or less than 8 wt. %, or less than 6 wt. %, or less than 5 wt. %, or less than 3 wt. %. In other embodiments, the composition definitively includes 9Z-retinal, wherein, relative to the total weight of retinoid component present, between 0.1-10 wt. %, or between 0.5 to 10 wt. %, or between 1 to 10 wt. %, or between 1.5 to 10 wt. % of 9Z-retinal; or between 0.1-8 wt. %, or between 0.5 to 8 wt. %, or between 1 to 8 wt. %, or between 1.5 to 8 wt. % of 9Z-retinal; or between 0.1-6 wt. %, or between 0.5 to 6 wt. %, or between 1 to 6 wt. %, or between 1.5 to 6 wt. % of 9Z-retinal; or between 0.1-5 wt. %, or between 0.5 to 5 wt. %, or between 1 to 5 wt. %, or between 1.5 to 5 wt. % of 9Z-retinal; or between 0.1-3 wt. %, or between 0.5 to 3 wt. %, or between 1 to 3 wt. %, or between 1.5 to 3 wt. % of 9Z-retinal.

Yet another potential constituent of the fermentative residue is β-carotene. The presence of β-carotene is believed to contribute to the more orange or peach-colored hue observed in fermentatively-produced retinoids when compared with their chemically-synthesized analogues. 3-carotenes impart health benefits of their own and as such their presence as fermentative residue in retinoid-containing compositions is not necessarily considered to be undesirable and may be desirable depending on the intended end-use application. In an embodiment, therefore, the fermentative residue comprises β-carotenes, wherein, relative to the total weight of retinoid component present, the β-carotenes are present in an amount of less than 4 wt. %, or less than 3 wt. %, or less than 2 wt. %, or less than 1 wt. %, or less than 0.5 wt. %.

In other embodiments of the first aspect, however, the composition comprises, relative to the total weight of retinoid component present, between 0-4 wt. %, or between 0.2-4 wt. %, or between 0.5-4 wt. %, or between 1-4 wt. % of β-carotenes, or
between 0-3 wt. %, or between 0.2-3 wt. %, or between 0.5-3 wt. %, or between 1-3 wt. % of β-carotenes, or
between 0-2 wt. %, or between 0.2-2 wt. %, or between 0.5-2 wt. %, or between 1-2 wt. % of β-carotenes, or
between 0-1 wt. %, or between 0.2-1 wt. %, or between 0.5-1 wt. % of β-carotenes, or
between 0-0.5 wt. %, or between 0.2-0.5 wt. % of β-carotenes.

Another potential fermentative residue constituent is farnesol. Farnesol has been identified as a potential skin irritant and as such, its presence in retinoid-containing compositions is not desirable. Therefore, it is preferable to limit the quantity of farnesol present in the retinoid-containing composition as much as is practical, such as below the limit of detection of many standard analytical techniques (such as less than 0.1 wt. % relative to the entire composition with which they are associated). In an embodiment, therefore, the composition comprises, relative to the total weight of retinoid component present, between 0-10,000 ppm, or between 2-10,000 ppm, or between 5-10,000 ppm, or between 10-10,000 ppm, or between 100-10,000 ppm of farnesol; or
between 0-5,000 ppm, or between 2-5,000 ppm, or between 5-5,000 ppm, or between 10-5,000 ppm, or between 100-5,000 ppm of farnesol; or
between 0-1,000 ppm, or between 2-1,000 ppm, or between 5-1,000 ppm, or between 10-1,000 ppm, or between 100-1,000 ppm of farnesol; or
between 0-100 ppm, or between 2-100 ppm, or between 5-100 ppm, or between 10-100 ppm of farnesol. In a particularly preferred embodiment, the composition is free from or substantially free from farnesol.

Another potential fermentative residue constituent includes dihydro-retinoids, including dihydro-retinol and/or dihydro-retinyl acetate, particularly in a range of 0.2 to 0.01 wt % or less, such as e.g. 0.2, 0.18, 0.16, 0.15, 0.14, 0.12, 0.1, 0.05, 0.01% or less, preferably a range of 0.2 to 0.1, 0.17 to 0.06, 0.1 to 0.05, 0.04 to 0.01, more preferably a percentage of 0.1% or less, all based on total ingredients within said total fermentatively-produced retinoid-containing composition. Also preferably, the percentage of dihydro-retinyl acetate is about 0.05% or less, particularly about 0.01% or less based on total ingredients within said composition.

Yet a further potential fermentative residue constituent is unconsumed feedstock or fermentation carbon source. As stated above, because it is desirable to maximize the circularity of the resulting retinoid composition from which such carbon source is produced, it is desirable to utilize biogenic carbon sources. A variety of carbon sources suitable in the fermentation of retinoids is described in WO2022090548A1, which is hereby incorporated by reference in its entirety as if set forth fully herein.

Specifically, suitable carbon sources to be used for the present invention might be selected from linear alkanes, free fatty acids, including triglycerides, particularly vegetable oil, such as e.g. selected from the group consisting of oil originated from corn, soy, olive, sunflower, canola, cottonseed, rapeseed, sesame, safflower, grapeseed or mixtures thereof, including the respective free fatty acids, such as e.g. oleic acid, palmitic acid or linoleic acid. Suitable carbon sources might furthermore be selected from ethanol, glycerol or glucose and mixtures of one or more of the above-listed carbon sources. In one embodiment, the present invention is directed to a process for the production of retinoids, in particular production of retinyl acetate, i.e. a two-phase culture system in the presence of a lipophilic solvent as defined herein, wherein a retinyl acetate producing host cell, preferably oleaginous yeast cell such as e.g. Yarrowia, is cultivated under suitable culture conditions, wherein the lipophilic solvent, preferably selected from solvents that are commercially available as Isopar™ fluids, particular selected from Isopar M, Isopar N, Isopar K, Isopar L, Isopar H or solvents with equivalent or identical properties but from other suppliers, is not consumed or evaporated during the fermentation process.

In an embodiment, the amount of fermentative carbon source is present, relative to the total weight of retinoid component present, in an amount of less than 2 wt. %, or less than 1 wt. %, or less than 0.75 wt. %. In various other embodiments, the fermentative carbon source is present, relative to the weight of retinoid component, between 0.05 to 2 wt. %, or between 0.1 to 2 wt. %, or between 0.2 to 2 wt. %; or between 0.05 to 1 wt. %, or between 0.1 to 1 wt. %, or between 0.2 to 1 wt. %; or between 0.05 to 0.5 wt. %, or between 0.1 to 0.5 wt. %, or between 0.2 to 0.5 wt. %.

As an additional source of fermentative residue, a so-called second phase solvent may be present. Otherwise known as lipophilic solvents, these may include isoparaffins comprising mixtures of alkanes, cyclo paraffin, isoalkanes, cycloalkanes, or dodecanes. The solvents might be natural or synthetic ones. Examples of commercially available useful solvents might be selected from Total, e.g. Isane® solvents, Shell, e.g. ShellSolTD or ShellSolT, Exxon Mobile, e.g. Isopar™ fluids, particularly such as e.g. Isopar M, Isopar N, Isopar H, Isopar K, Isopar L, or mixtures thereof or mixtures with isododecane isomers, as e.g. commercially available under the tradename AC365770010 (Acros Organics). Preferably, the second phase solvent is selected from isoparaffins comprising, e.g. Isopar M, Isopar N, Isopar H, Isopar K, Isopar L, and mixtures thereof, more preferably comprising Isopar N, Isopar L and/or Isopar M.

Further suitable lipophilic solvents, i.e. second phase solvents, with minimal loss and/or disappearance during the fermentation as defined herein and to be used for the present invention might be selected from lipophilic solvents comprising mixtures of n-alkanes, isoalkanes, hydrocarbons. The solvents might be natural or synthetic ones. Examples of commercially available useful solvents might be selected from Exxon Mobil, as e.g. commercially available under the tradenames Exxsol D60, D80, D95 or D110.

It is understood that useful solvents include the above listed commercially available solvents as well as the respective solvents with the same or equivalent properties but known/available from other suppliers.

As used herein, a solvent has equivalent or identical properties as Isopar fluids, including Isopar M, Isopar H, Isopar K, Isopar L, and is defined as branched-chain isomers, preferably terminally methylated form of a straight-chain alkane, which contain six to twenty six carbons, perhaps chemically coupled from smaller alkane precursors in strong acid, hydrogenated with H2 and catalyst, such as nickel or platinum, to remove unsaturation and trace aromatics. These are known in current industrial suppliers as Isopar (Exxon Mobil Chemical), Soltrol (Chevron Phillips Chemical Company), Shellsol OMS (Royal Dutch Shell), isooctane and iso-dodecane. Specifically, the use of Isopar M is preferred. The use of these in consumer products is outlined in a review by Johnson et al., Int J Toxicol. 2012 November-December; 31(6 Suppl):269S-95S. As used herein, a solvent has equivalent or identical properties as Exxsol D60, D80, D95, D110. These Exxsol Ds are narrow boiling distillation cuts from cracked hydrocarbons that have been reduced by catalytic hydrogenation to remove aromatics and unsaturation.

In an embodiment, therefore, the fermentative residue comprises an isoparaffinic fluid. In various related embodiments, the isoparaffinic fluid is present, relative to the weight of the entire retinoid component (i) and fermentative residue thereof (ii), in an amount of less than 3 wt. %, or less than 2 wt. %, or less than 1.5 wt. %, or less than 1.25 wt. %, or wherein the isoparaffinic fluid is present, relative to the weight of the entire retinoid component (i) and fermentative residue thereof (ii), in an amount of 0.1-2 wt. %, or between 0.2-2 wt. %, or between 0.5-2 wt. %, or between 0.1-1.5 wt. %, or between 0.2-1.5 wt. %, or between 0.5-1.5 wt. %. In a preferred embodiment, if present, the isoparaffinic fluid comprises, consists of, or consists essentially of Isopar M.

Fermentatively-produced compositions of the first aspect are further tuned so as to have certain quantities of (i) and (ii) relative to each other. That is, in all embodiments of the first aspect, the retinoid-containing compositions are configured such that the retinoid component is present, relative to the fermentative residue thereof, in a ratio by weight of greater than 4:1. Thus it is desirable that the “active” ingredient; i.e. the retinoid component—which is preferably retinol, retinyl acetate, or retinyl palmitate—is maintained at a very high level relative to the fermentative residue thereof. Therefore, in other embodiments, that ratio by weight of retinoid component to fermentative residue thereof is tuned to be greater than 5:1, or greater than 6:1, or greater than 10:1, or greater than 20:1, or even greater than 25:1.

However, inventors have still surprisingly identified that the presence of the fermentative residue, and in particular specific types, quantities, and ratios of fermentative residue constituents as described elsewhere herein, supra, are still associated with beneficial effects into the composition with which they are associated, such as excellent oxidative stability and/or antimicrobial efficacy. As will be described below, such benefits make the associated fermentatively-produced retinoid-containing compositions particularly suitable for use in a variety of formulation techniques, such as spray-drying or extrusion. This then facilitates their use in a variety of food, feed, pharmaceutical, or personal care products or pre-products (hereinafter “(pre)products”). Therefore, in other embodiments, the retinoid component—which is preferably retinyl acetate, retinol, or retinyl palmitate—is present, relative to the fermentative residue thereof,
in a ratio by weight between 4:1 to 100:1, or between 5:1 to 100:1, or between 6:1 to 100:1, or between 10:1 to 100:1, or between 20:1 to 100:1, or between 25:1 to 100:1; or
in a ratio by weight between 4:1 to 50:1, or between 5:1 to 50:1, or between 6:1 to 50:1, or between 10:1 to 50:1, or between 20:1 to 50:1, or between 25:1 to 50:1; or
in a ratio by weight between 4:1 to 35:1, or between 5:1 to 35:1, or between 6:1 to 35:1, or between 10:1 to 35:1, or between 20:1 to 35:1, or between 25:1 to 35:1; or
in a ratio by weight between 4:1 to 30:1, or between 5:1 to 30:1, or between 6:1 to 30:1, or between 10:1 to 30:1, or between 20:1 to 30:1, or between 25:1 to 30:1.

Although it has not previously been appreciated that the content, ratios, and types of fermentative residue profiles as described herein are associated with one or more beneficial effects, one appreciated, the skilled artisan to which this invention relates can tune or configure such fermentative residue profiles as appropriate by way of a number of known methods and techniques. Specifically, the micro-organism(s) utilized in the fermentation process can be selected and/or engineered to produce certain desired retinoid components and/or fermentative residues, as is described elsewhere herein. Also, the fermentation conditions can also be modified accordingly, which further includes the selection and quantity of various additives (i.e. anti-foaming agents) or even the nature, type, and quantity of the carbon source and/or lipophilic solvents used. Yet further, the skilled artisan can readily tune the fermentative profile by virtue of the nature and number of downstream processing steps, such as conditions for distillation, crystallization, filtration, and or washing. The fermentation and isolation conditions are further described elsewhere herein, infra.

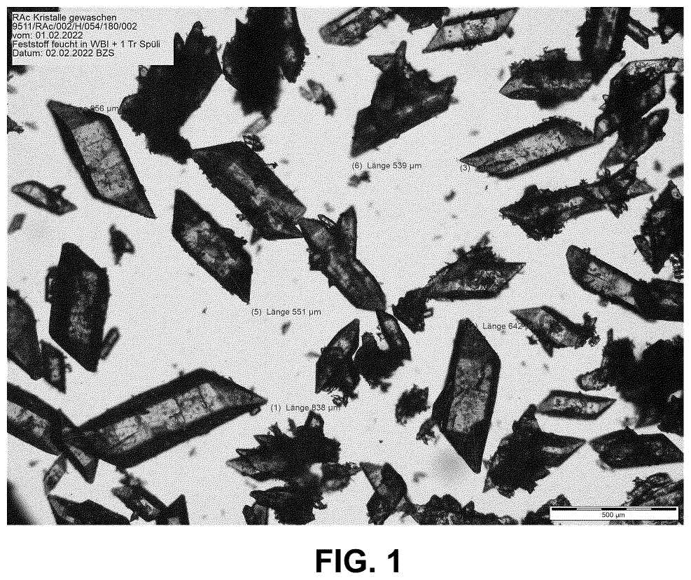

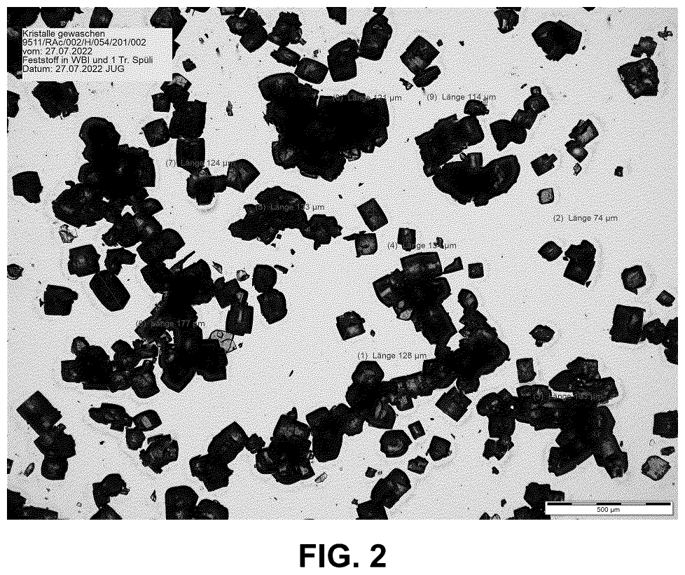

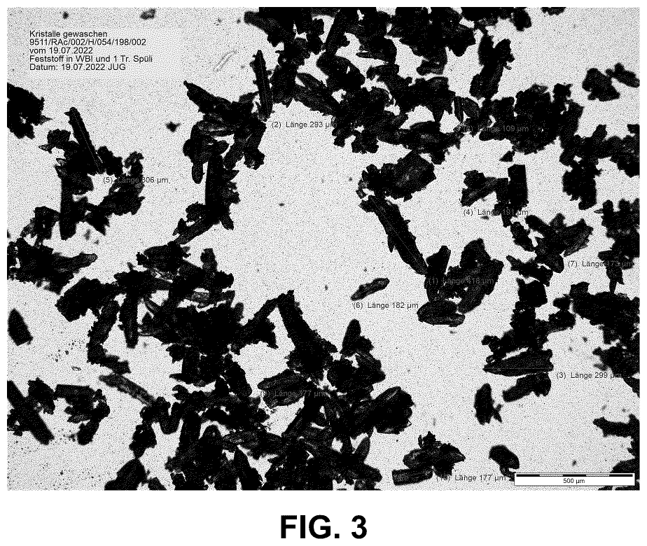

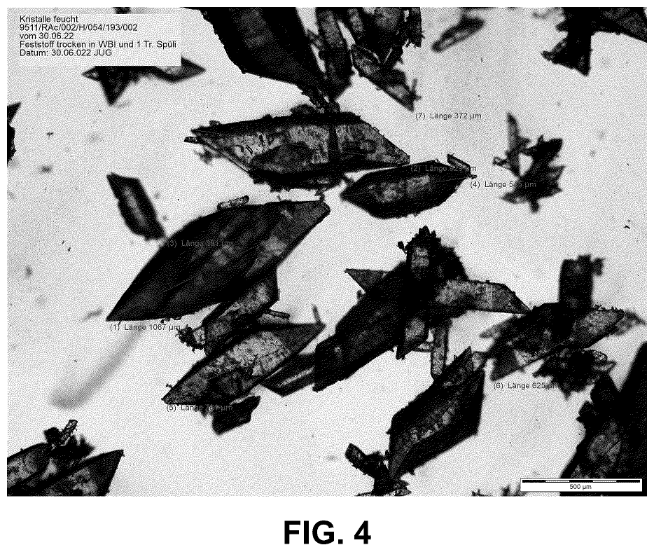

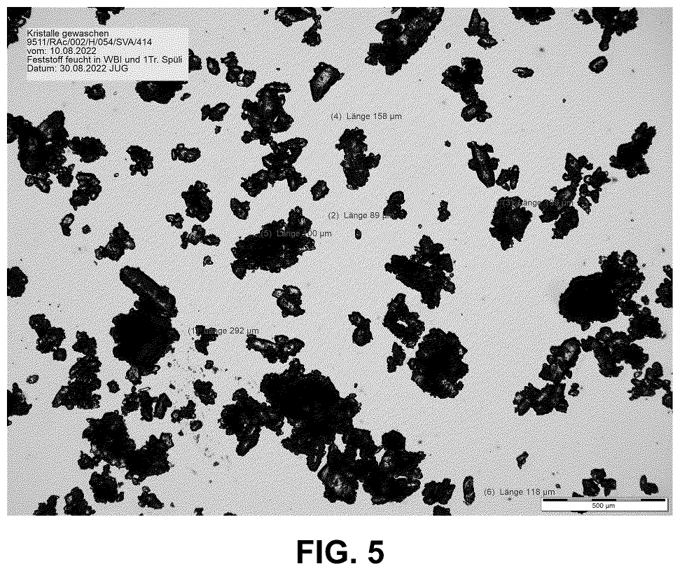

The retinoid-containing composition according to the first aspect may be provided in any suitable form, however it is preferably created in a way so as to maximize ease of handling and transport. In an embodiment, the retinoid-containing composition according to the first aspect is provided in a crystalline form, or in a predominantly crystalline form. As intended herein, crystalline form indicates that the composition is composed of solid materials or particles whose constituents are arranged in a highly ordered microscopic structure, forming a crystal lattice that extends in all directions. Inventors have discovered that the fermentative residue profile may have an influence on the crystal structure formed. A variety of retinoid-containing compositions in crystalline form are depicted in FIGS. 1-5. FIG. 1, FIG. 2, FIG. 3, and FIG. 4, respectively, depict the crystalline form of the fermentatively-produced retinoid-containing compositions of examples 1-4 used in the examples below. Therein, crystals are determined to be on the order of 200-900 microns or larger and of an elongated rhomboid shape such as in FIG. 1, FIG. 3, or FIG. 4, or of a more cuboid shape with crystal lengths on the order or 100-200 microns such as is depicted in FIG. 2. FIG. 5, by contrast, shows the significantly different crystalline structure that results when a chemically synthesized retinoid-containing composition not possessing a fermentative residue is used, with crystals in such case typically on the order of 100-200 microns or less. As can be seen, the average crystal size or length, as determined specifically by computer-automated micrographic analysis, varies between the different fermentatively-produced examples and the chemical analogue. Inventors believe, without wishing to be bound by any theory, that the crystal size can influence the oxidative stability of the associated retinoid-containing composition.

In certain embodiments, therefore, the fermentatively-produced retinoid-containing composition is present in crystalline form, wherein the crystalline form comprises a plurality of crystals having an average particle length D₅₀ as determined by a microscopic imaging method, of greater than 100 microns, or greater than 200 microns, or greater than 300 microns, or greater than 400 microns, or between 100-1200 microns, or between 100-1000 microns, or between 100-800 microns, or between 200-1200 microns, or between 200-1000 microns, or between 200-800 micrometers.

The form in which the fermentatively-produced retinoid component appears may be as a so-called “dry crystal” or “wet crystal”. In a dry crystalline form, the composition is generally lacking any liquid component and generally behaves and appears as a granular or powder composition. In a wet crystalline form, the composition comprises a large amount of crystals, but also a residual appreciable quantity of a liquid, such as an associated solvent. As such it may appear and behave as a slurry.

In yet another form, the composition containing the retinoid (i) and fermentative residue (ii) are dissolved, mixed, and/or (most preferably) emulsified in an oil. In a preferred embodiment, if not present in crystalline form, the retinoid component (i) and fermentative residue thereof (ii) are emulsified in a stabilized oil. This stabilized oil emulsion preferably comprises any suitable oil, preferably one that is a triglyceride, such as, without limitation, a vegetable oil and/or fat. It will be understood in this sense that the oil will not be included as part of the fermentative residue component (ii).

As a suitable stabilizing agent, the stabilized emulsion preferably also comprises a fat-soluble antioxidant, and may comprise, without limitation, an ascorbic acid or salts thereof, tocopherol, butylated hydroxytoluene (BHT), butylated hydroxyanisole (BHA), propyl gallate, tert. butyl hydroxyquinoline, ethoxyquin and/or ascorbic acid esters of a fatty acid. It will be understood in this sense that the stabilizing agent will not be included as part of the fermentative residue component (ii).

As stated, compositions according to the first aspect are preferably configured to impart advantageous properties, such as excellent oxidative stability. Accordingly, when one or more compositions configured as described above are utilized, wherein the amount, measured relative to the weight of the entire composition, of retinoid component (preferably a retinyl acetate, retinol, or retinyl palmitate) is within 5 wt. %, or within 4 wt. %, or within 3 wt. %, of the amount of said retinoid component present in the composition after it is subjected to a heat stability test whereby the composition is heated to 105 degrees Celsius for 3 hours, according to a method described elsewhere herein.

A second aspect of the invention is a method for fermentatively producing a retinoid-containing composition, the method comprising the steps of:
(a) cultivating a microorganism under conditions that allow for production of a fermentation product;
(b) isolating the fermentation product to yield an isolated retinoid-containing composition;
wherein the isolated retinoid-containing composition comprises
   (i) a retinoid component comprising a mixture of cis-isomers and trans-isomers, and
   (ii) and a fermentative residue thereof;
   wherein, the retinoid component is present, relative to the fermentative residue thereof, in a ratio by weight of greater than 4:1,
wherein the cis-isomers are present by weight, relative to the weight of the entire retinoid component, in an amount of less than 3 wt. %.

Embodiments according to the second aspect of the invention related to fermentation methods of producing a retinoid-containing composition. In all such embodiments, the method first involves the step of cultivating a microorganism under conditions allowing for production of the desired fermentation product, which preferably includes the retinoid component as described and used in all embodiments of the first aspect of the invention.

Any suitable micro-organism may be used, as will be appreciated by the skilled artisan. Particularly suitable microorganisms include fungi, such as those of the Yarrowia genus, which are oleaginous, in that they are capable of accumulating lipids to at least 20% of their dry cell weight; and further, possibly as a result of genetic engineering, such microorganisms are capable of producing the desired product, which they can preferably accumulate to at least 1% of its dry cell weight. General methods for the cultivation of carotenoids (a precursor to retinoids) are described in U.S. Pat. No. 7,851,199, assigned to DSM IP Assets BV, which is hereby incorporated by reference in its entirety as if set forth fully herein. Fermentative production of isoprenoids, a broader class of compounds which include retinoids, is described in WO2022090548, which is assigned to DSM IP Assets BV, and which is also hereby incorporated by reference in its entirety as if set forth fully herein.

Those of ordinary skill in the art will readily appreciate that a variety of yeast and fungal strains exist that are naturally oleaginous or that naturally produce desired retinoids. Any of such strains may be utilized as host strains according to second aspect of the present invention, and may be engineered or otherwise manipulated to generate inventive oleaginous, particularly retinoid-producing strains. Alternatively, strains that naturally are neither oleaginous nor retinoid-producing may be employed. Furthermore, even when a particular strain has a natural capacity for oleaginy or for retinoid production, its natural capabilities may be adjusted as described herein, so as to change the production level of lipid and/or retinoid. In certain embodiments engineering or manipulation of a strain results in modification of a type of lipid and/or retinoid which is produced. For example, a strain may be naturally oleaginous and/or retinogenic, however engineering or modification of the strain may be employed so as to change the type of lipid which is accumulated and or to change the type of retinoid which is produced.

When selecting a particular yeast or fungal strain for use in accordance with the second aspect of the present invention, it will generally be desirable to select one whose cultivation characteristics are amenable to commercial scale production. For example, it will generally (though not necessarily always) be desirable to avoid filamentous organisms, or organisms with particularly unusual or stringent requirements for growth conditions. However, where conditions for commercial scale production can be applied which allow for utilization of filamentous organisms, these may be selected as host cells. In some embodiments of the invention, it will be desirable to utilize edible organisms as host cells, as they may optionally be formulated directly into food or feed additives, or into nutritional supplements, as desired. For ease of production, some embodiments of the invention utilize host cells that are genetically tractable, amenable to molecular genetics (e.g., can be efficiently transformed, especially with established or available vectors; optionally can incorporate and/or integrate multiple genes, for example sequentially; and/or have known genetic sequence; etc), devoid of complex growth requirements (e.g., a necessity for light), mesophilic (e.g., prefer growth temperatures within the range of about 25-32° C.), able to assimilate a variety of carbon and nitrogen sources and/or capable of growing to high cell density. Alternatively or additionally, various embodiments of the invention utilize host cells that grow as single cells rather than multicellular organisms (e.g., as mycelia).

In general, when it is desirable to utilize a naturally oleaginous organism in accordance with the second aspect of the present invention, any modifiable and cultivatable oleaginous organism may be employed. In certain embodiments of the invention, yeast or fungi of genera including, but not limited to, Blakeslea, Candida, Cryptococcus, Cunninghamella, Lipomyces, Mortierella, Mucor, Phycomyces, Pythium, Rhodosporidium, Rhodotorula, Trichosporon, and Yarrowia are employed. In certain particular embodiments, organisms of species that include, but are not limited to, Blakeslea trispora, Candida pulcherrima, C. revkaufi, C. tropicalis, Cryptococcus curvatus, Cunninghamella echinulata, C. elegans, C. japonica, Lipomyces starkeyi, L. lipoferus, Mortierella alpina, M. isabellina, M. ramanniana, M. vinacea, Mucor circinelloides, Phycomyces blakesleanus, Pythium irregulare, Rhodosporidium toruloides, Rhodotorula glutinis, R. gracilis, R. graminis, R. mucilaginosa, R. pinicola, Trichosporon pullans, T. cutaneum, and Yarrowia lipolytica are used.

Those of ordinary skill in the art will appreciate that the selection of a particular host cell for use in accordance with embodiments of the second aspect of the present invention will also affect, for example, the selection of expression sequences utilized with any heterologous polypeptide to be introduced into the cell, and will also influence various aspects of culture conditions, etc. Much is known about the different gene regulatory requirements, protein targeting sequence requirements, and cultivation requirements, of different host cells to be utilized in accordance with the second aspect of the present invention (see, for example, with respect to Yarrowia, Barth et al. FEMS Microbiol Rev. 19:219, 1997; Madzak et al. J Biotechnol. 109:63, 2004; see, for example, with respect to Xanthophyllomyces, Verdoes et al. Appl Environ Microbiol 69: 3728-38, 2003; Visser et al. FEMS Yeast Res 4: 221-31, 2003; Martinez et al. Antonie Van Leeuwenhoek. 73(2):147-53, 1998; Kim et al. Appl Environ Microbiol. 64(5):1947-9, 1998; Wery et al. Gene. 184(1):89-97, 1997; see, for example, with respect to Saccharomyces, Guthrie and Fink Methods in Enzymology 194:1-933, 1991). In certain aspects, for example, targeting sequences of the host cell (or closely related analogs) may be useful to include for directing heterologous proteins to subcellular localization. Thus, such useful targeting sequences can be added to heterologous sequence for proper intracellular localization of activity. In other aspects (e.g., addition of mitochondrial targeting sequences), heterologous targeting sequences may be eliminated or altered in the selected heterologous sequence (e.g., alteration or removal of source organism plant chloroplast targeting sequences).

Embodiments of the second aspect also involve the step of isolating the fermentation product to yield an isolated retinoid-containing composition. As discussed above, accumulation of lipid bodies in oleaginous organisms is generally induced by growing the relevant organism in the presence of excess carbon source and, e.g., limiting nitrogen. Specific conditions for inducing such accumulation have previously been established for a number of different oleaginous organisms (see, for example, Wolf (ed.) Non-conventional yeasts in biotechnology Vol. 1, Springer-Verlag, Berlin, Germany, pp. 313-338; Lipids 18(9):623, 1983; Indian J. Exp. Biol. 35(3):313, 1997; J. Ind. Microbiol. Biotechnol. 30(1):75, 2003; Bioresour Technol. 95(3):287, 2004, each of which is incorporated herein by reference in its entirety).

Methods and systems for isolating lipid bodies have been established for a wide variety of oleaginous organisms (see, for example, U.S. Pat. Nos. 5,164,308; 5,374,657; 5,422,247; 5,550,156; 5,583,019; 6,166,231; 6,541,049; 6,727,373; 6,750,048; and 6,812,001, each of which is incorporated herein by reference in its entirety). In brief, cells are typically recovered from culture, often by spray drying, filtering or centrifugation. In some instances, cells are homogenized and then subjected to supercritical liquid extraction or solvent extraction (e.g., with solvents such as chloroform, hexane, methylene chloride, methanol, isopropanol, ethyl acetate, etc.), yielding a crude oil suspension. This oil suspension may optionally be refined as known in the art. Refined oils may be used directly as feed or food additives. Alternatively or additionally, retinoids can be isolated from the oil using conventional techniques.

Given the sensitivity of retinoids generally to oxidation, many embodiments of the invention employ oxidative stabilizers (e.g., tocopherols, vitamin C; ethoxyquin; vitamin E, BHT, BHA, TBHQ, etc, or combinations thereof) during and/or after retinoid isolation. Alternatively or additionally, microencapsulation, for example with proteins, may be employed to add a physical barrier to oxidation and/or to improve handing.

Isolation of the fermentation product may further comprise one or more additional downstream processing steps. Among these steps include, without limitation, various processes and techniques for crystallization, distillation, filtration, and/or washing. Such processes are known and will be appreciated by the person of ordinary skill in the art to which this invention applies.

Regardless of the specific type and nature of both the cultivating (a) and isolating step (b) according to embodiments of the second aspect, the process is configured so as to yield a resulting retinoid-containing composition which possesses (i) a retinoid component comprising a mixture of cis-isomers and trans-isomers, and (ii) and a fermentative residue thereof, wherein, the retinoid component is present, relative to the fermentative residue thereof, in a ratio by weight of greater than 4:1, and wherein the cis-isomers are present by weight, relative to the weight of the entire retinoid component, in an amount of less than 3 wt. %. It will be appreciated that the retinoid-containing composition produced in embodiments of the second aspect will be preferably according to any one of the embodiments described above with respect to the first aspect of the invention.

Optionally, the methods according to the second aspect also include the step of combining the isolated retinoid-containing composition, retinoid-containing composition, the dry retinoid crystal composition, and/or the isolated retinol product with one or more other additives. This may be done to create a stabilized formulation for subsequent transport—a further description of certain non-limiting processes for which are described below—but also to create a (pre)product for a food, feed, pharmaceutical, or personal care application.

A third aspect of the invention is a method for preparing a food, feed, pharmaceutical, or personal care (pre)product comprising the steps of:
(a) providing a composition of matter comprising, consisting of, or consisting essentially of
   (i) a retinoid component comprising a mixture of cis-isomers and trans-isomers; and
   (ii) a fermentative residue thereof; wherein, the retinoid component is present, relative to the fermentative residue thereof, in a ratio by weight of greater than 4:1; wherein the cis-isomers are present by weight, relative to the weight of the entire retinoid component, in an amount of less than 3 wt. %; optionally also wherein a bio-based carbon content of (i) and (ii) is greater than 50%, or greater than 60%, or greater than 70%, or greater than 90%; and
(b) formulating the composition of matter to produce a formulation, preferably for use as a food, feed, pharmaceutical, or personal care (pre)product.

According to embodiments of the third aspect, the composition of matter provided comprises, consists of, or consists essentially of (i) a retinoid component comprising a mixture of cis-isomers and trans-isomers; and (ii) a fermentative residue thereof, wherein, the retinoid component is present, relative to the fermentative residue thereof, in a ratio by weight of greater than 4:1; wherein the cis-isomers are present by weight, relative to the weight of the entire retinoid component, in an amount of less than 3 wt. %; optionally also wherein a bio-based carbon content of (i) and (ii) is greater than 50%, or greater than 60%, or greater than 70%, or greater than 90%. Preferably, the composition provided is any of the fermentatively-produced retinoid-containing compositions according to any of the embodiments of the first aspect of the invention, and preferably also which were provided by virtue of any of the methods described in any of the embodiments of the second aspect.

With the composition provided, it is often desirable to formulate such composition of matter in order to increase its suitability for storage, handling and transport, bioavailability, miscibility, or other properties for subsequent suitability—whether with (pre-product) or without (product) one or more other additives or ingredients—for use in a variety of end-use applications. As such, the formulation techniques described according to embodiments of this third aspect preferably impart improved stability and/or suitability for use as a food, feed, pharmaceutical, or personal care (pre)product.

A variety of formulation techniques are well known in the art to which this invention applies. The typical goal of such techniques is to incorporate the primary desired ingredient (in this case the retinoid component) into stabilized emulsions or suspensions in a variety of ways. One known type of formulation technology involves micro-encapsulation. Micro-encapsulation techniques are known and described generally, and may include, without limitation, spray drying, beadlet technology, coacervation, or spray-chilling. Certain micro-encapsulation technologies are described in U.S. Pat. No. 8,765,186B2, which is hereby incorporated by reference in its entirety as if set forth fully herein.

In an embodiment, the method of the third aspect involves a beadlet formulation process. In an embodiment, therefore, the method for preparing a food, feed, pharmaceutical, or personal care (pre)product according to the third aspect involves as associated within the formulating step (b), the following sub-steps:
(1) dissolving a matrix component in water to produce a matrix solution;
(2) heating the composition of matter according to any of the previous claims optionally preferably also in an oil to yield an active phase;
(3) emulsifying the active phase into the matrix solution to obtain a dispersion; and
(4) drying the dispersion in the presence of a catch medium to obtain at least one beadlet.

In the dissolving sub-step, a matrix material is typically dissolved in water to create a matrix solution. This will form the medium into which the micro-encapsulated active beadlets will form. Any suitable matrix component may be used, such as lignosulfonate or hydrocolloids. The term “hydrocolloid” as used herein includes gelatin, xanthan gum, acacia gum, pectins, guar, caroub gums, alginates, celluloses, cellulose derivatives, such as carboxymethylcellulose, and/or modified polysaccharides. The term “modified polysaccharide” as used herein relates to polysaccharides which contain a lipophilic moiety, e.g. a hydrocarbon moiety having a chain length of preferably 5 to 18 carbon atoms in the straight chain. Preferably the modified polysaccharide should be acceptable for human consumption, i.e. preferred modified polysaccharides should be GRAS (generally recognized as safe) or approved for food consumption as determined by the various regulatory agencies worldwide. A preferred modified polysaccharide is modified food starch.

The term “modified food starch” as used herein relates to modified starches that are made from starches substituted by known chemical methods with hydrophobic moieties. For example starch may be treated with cyclic dicarboxylic acid anhydrides such as succinic and/or glutaric anhydrides, substituted with an alkyl or alkenyl hydrocarbon group. According to certain embodiments, the preferred starch includes a starch sodium octenyl succinate (“OSA”).

OSA-starches may contain further hydrocolloids, such as starch, maltodextrin, carbohydrates, gum, corn syrup etc. and optionally any typical emulsifier (as co-emulgator), such as mono- and diglycerides of fatty acids, polyglycerol esters of fatty acids, lecithins, sorbitan monostearate, and plant fibre or sugar. OSA-starches are commercially available e.g. from National Starch under the trade names HiCap 100, Capsul, Capsul HS, Purity Gum 2000, UNI-PURE, NYLON VII; from Roquette Freres; from CereStar under the tradename C*EmCap or from Tate & Lyle.

The terms “modified polysaccharides”, “modified starches” and “OSA-starches” encompass further also modified polysaccharides/modified starches/OSA-starches that were partly hydrolysed enzymatically, e.g. by glycosylases (EC 3.2; see [[http://www.]]chem.qmul.ac.uk/iubmb/enzyme/EC3.2[[/]]), as well as to modified poly-accharides/modified starches/OSA-starches that were partly hydrolysed chemically by known methods.

In other embodiments, the matrix component may comprise modified (food) starches, pectin, alginate, carra-geenan, furcellaran, chitosan, maltodextrin, dextrin derivatives, celluloses and cellulose derivatives (e.g. cellulose acetate, methyl cellulose, hydroxypropyl methyl cellulose), ligno-sulfonate, polysaccharide gums (such as gum acacia, gum arabic, flaxseed gum, ghatti gum, tamarind gum and arabino-galactan), gelatin (bovine, fish, pork, poultry), plant proteins (such as are for example peas, soybeans, castor beans, cotton, potatoes, sweet potatoes, manioc, rapeseed, sunflowers, sesame, linseed, safflower, lentils, nuts, wheat, rice, maize, barley, rye, oats, lupin and sorghum), animal proteins including milk or whey proteins, lecithin, polyglycerol ester of fatty acids, monoglycerides of fatty acids, diglycerides of fatty acids, sorbitan ester, PG ester and sugar ester (as well as derivatives thereof).

The heating sub-step involves heating a composition of matter, preferably also in an oil, to yield an active phase. The composition of matter utilized in this step is preferably a fermentatively-produced retinoid-containing composition according to any of the embodiments of the first aspect of the invention, as described elsewhere herein, supra.

Any suitable oil may be used, although it is highly preferred that the oil employed is compatible with the composition of matter with which it is to be incorporated. In a preferred embodiment, the oil is a triglyceride, optionally selected from vegetable oils and/or fats, such as corn oil, sunflower oil, (hydrogenated) soybean oil, safflower oil, rape seed oil, peanut oil, (hydrogenated) palm oil, palm kernel oil, cotton seed oil and/or coconut oil, including fractionated qualities thereof. The triglycerides can preferably be so-called MCT (medium chain triglycerides), i.e. ester of medium chain fatty acids (preferably saturated fatty acids with a chain length of 6 to 12 C atoms) and glycerol. Preferred triglycerides are corn oil, sunflower oil, (hydrogenated) soybean oil and/or (hydrogenated) palm oil.

According to certain embodiments, it is useful to include a fat-soluble anti-oxidant, as this will further limit degradation and/or oxidation of the active “retinoid component” during and after the formulation process. The fat-soluble antioxidant may be incorporated at any suitable time, such as during sub-step (2) of heating the composition of matter and oil. Any suitable fat-soluble anti-oxidant may be employed, such as an ascorbic acid or salts thereof, tocopherol, butylated hydroxytoluene (BHT), butylated hydroxyanisole (BHA), propyl gallate, tert. butyl hydroxyquinoline, ethoxyquin and/or ascorbic acid esters of a fatty acid.

The quantities and ratios of the aforementioned matrix component, water, composition of matter, oil, and fat-soluble antioxidant as described above may be modified as desired and as will be appreciated by the skilled person to which this invention applies.

With the matrix solution and active phase provided, it becomes necessary to emulsify the active phase into the matrix solution to obtain a dispersion. This may be accomplished via a variety of techniques and methods, such as those described in U.S. Pat. No. 8,765,186 by way of non-limiting example.

The dispersion may optionally further be processed to assure a desired droplet size or distribution, and may be fed through, e.g., a homogenizer to accomplish this. Thereafter the dispersion is preferably pump-fed, to an apparatus or series of apparatuses to remove any solvent and dry the dispersion.

The drying step may be carried out with any conventional drying process known to the person skilled in the art, preferred are freeze-drying, spray drying, spray drying in combination with fluidised bed granulation (commonly known as fluidised spray drying or FSD) and/or a powder catch process where sprayed emulsion or dispersion droplets are caught in a bed of an absorbant such as starch or calcium silicate or silicic acid or calcium carbonate or mixtures thereof and subsequently dried.

It is advantageous if the residual moisture content in the powder obtained by the drying sub-step is in the range of from 0.5 to 7.0 weight-%, preferably from 4 to 6 weight-%, each based on the total weight of the material. If utilized, freeze-drying is preferably performed at a temperature of from −20° C. to −50° C. for 10 to 48 hours. In a preferred embodiment the emulsion or dispersion obtained/obtainable by the process of such embodiments of the third aspect of the present invention are spray-dried. In this case it is preferred to select the spray drying parameters as follows:
Air inlet: from about 100° C. to about 250° C., preferably of from 150° C. to about 200° C., more preferably of from about 160° C. to about 190° C.;
Air exit: from about 45° C. to about 160° C., preferably of from about 55° C. to about 110° C., more preferably of from about 65° C. to about 95° C. The solid form may be further dried and/or granulated via a variety of other known techniques.

Preferably, the drying process includes spraying the dispersion into a vessel that includes a catch medium. The catch medium traps the dispersion particles and surrounds them, effectively facilitating the drying even further. The benefit of the catch medium is that drying can be accomplished at lower temperatures and with lower energy input than traditional spray-drying techniques.

A preferred catch medium is corn starch, although any other suitable catch medium can be used depending upon the specific nature of the active retinoid component and the desired end-use application. When the method involves the sub-step of drying the dispersion in the presence of the catch medium, the resulting product is known as a beadlet. Technologies which produce beadlets are known to yield formulations with high-levels of stability, optimized handling, and good dissolution characteristics.

Inventors have discovered that the compositions described in the embodiments of the first aspect, and/or those created by the embodiments of the second aspect are particularly suitable for use in such beadlet-generating processes, not least of which because of the excellent oxidative stability characteristics they possess.

Another formulation technique which may employed according to other embodiments of the third aspect of the invention involves extrusion. Extrusion techniques are known generally, and are described in, i.a, WO2014083065, which is hereby incorporated by reference in its entirety as if set forth fully herein. Other exemplary publications include WO 2007/055815 A1, WO 2021/163836 A1, WO 2014/083065 A1, WO 2017/005622 A1 and WO 2019/175326 A1. A main advantage of using extrusion technology is that high viscous solutions can be formulated and less water can be used for the dispersion, which then requires less drying. Furthermore, an extrusion process can be run as a continuous process. It can be found in the prior art that emulsions comprising fat soluble vitamins are extruded. US 2004/0201116 discloses pellets which are obtained by a combination of producing emulsions using devices like high pressure homogenizers with subsequent direct pelleting or extrusion as a second process step.

According to embodiments wherein such so-called extrusion methods are used, the formulating step (b) of the third aspect involves the following sub-steps:
(1) introducing the composition of matter according to any of the previous claims, a matrix component, water, and an oil into an extruder having an orifice; and
(2) extruding said composition of matter, matrix component, water, and oil through the orifice to yield an extrudate.

The introduction sub-step (1) requires introduction of a composition, preferably any of the fermentatively-produced retinoid-containing compositions according to any of the embodiments of the first aspect and/or any such compositions produced via an of the embodiments of the second aspect of the invention, alongside a matrix component, oil, and water. The matrix components and oil may be selected from any suitable types, including those described above with respect to beadlet technology embodiments of the third aspect. As was described above with respect to other micro-encapsulation techniques, an oil-in-water/matrix emulsion or dispersion should be created amongst said components.

The emulsion or dispersion can be formed prior to incorporation into the extruder as a pre-mixed active phase, or alternatively—as has been described in WO2014083065—inside the extruder apparatus itself. Thus, the introduction of the composition or “active”, oil, water, and matrix may be performed tougher or in four different steps at up to four different locations as will be deemed appropriate. Inventors have surprisingly discovered that the fermentatively-produced retinoid-containing compositions according to the first aspect and/or those produced via methods according to the second aspect are particularly suitable for extrusion methods wherein the emulsion is formed inside the extruder itself. Without wishing to be bound by any theory, Inventors theorize that this may be due to the excellent oxidative stability of such compositions.

In sub-step (2), the introduced materials are extruded through the extruder, eventually through an orifice, to yield an extrudate. In a non-limiting embodiment, the extruder apparatus comprises an extrusion die having circular orifices for material exit. Behind the die, the apparatus typically comprises one or more feeds to provide material into a barrel, where one, two or multiple screws, driven by a mechanical drive (e.g. an electric, or hydraulic, or pneumatic motor), mix the material, convey it towards the die and build up pressure. Optionally, means for heating and/or cooling the material can be disposed at some point of the barrel. The socket for holding the die can also be heated or cooled. When in operation, extruded material exits the orifices as strands of particular cross section. The emulsion or suspension comprising the composition of matter, matrix component, water, and oil remains in a viscous state during extrusion and solidifies through cooling and/or drying after it exits the extrusion die.

After extrusion, it is preferable also to incorporate sub-step (3) of cutting the extrudate into discrete particles, and (4) optionally, drying the discrete particles. For the avoidance of doubt, these steps do not need to be performed sequentially and may be performed simultaneously; indeed some quantum of drying preferably occurs not only after cutting, but also preferably before and during such process as well.

In a preferred embodiment, at the exit of the extrusion die and/or the orifice, a cutter cuts pellets off the extrudate. In an embodiment, a lateral flow of transportation fluid, typically transportation air, carries away the pellets from the die, however, the mass is often still viscous and sticky, especially in the core of the strand, despite the cooling and drying effect of the transportation fluid. This leads to a clogging of machine parts, especially cutter and extrusion die, and is detrimental to process stability and maintenance frequency. It also leads to agglomeration of product particles itself and is detrimental to product quality. Depending on the kind of mass and the shape and size of the extrudate, adjusting temperature and flow speed of the transportation air or cooling of the knives of the cutter by separate streams of cooling air may or may not always satisfactorily solve the problem.

Thus, the drying step may further include a means for providing an additional vertical flow of cooling air that exits an outlet to increase the cooling and drying speed of the extrudate, when compared to the cooling and drying by transportation air alone, in particular at and around the core of an extrudate. Further, it has an additional cooling effect on the knives of the cutter, when compared to the cooling by cooling air that exits outlet alone.

These effects of the cooling air that exits such an outlet can be of particular advantage in the extrusion of oil-in-water emulsions comprising fat-soluble nutritional or pharmaceutical actives dissolved in oil particles of the emulsion. The emulsions usually comprise a thickening or gelling agent, which renders them quite sticky when exiting the die at elevated temperature, and the vertical cooling air flow directly onto the face of the exiting material strand reduces stickiness and the tendency of the apparatus to clog. Accordingly, in an embodiment, the formulating step (b) further comprises a step of directing a flow of cooling fluid onto the orifice and the extrudate. In an embodiment, this directing step is implemented such that the cooling fluid is configured to flow along a cooling flow axis, and the extrudate is configured to be extruded through an exit axis, wherein the cooling flow axis and the exit axis are substantially anti-parallel and/or substantially colinear.

A fourth aspect of the invention is a beadlet or extrudate produced by the method of any of the embodiments of the third aspect of the invention and/or which incorporate the composition of matter according to any of the embodiments of the first aspect of the invention and/or made by any of the embodiments according to the second aspect of the invention.

A fifth aspect of the invention is a food, feed, pharmaceutical, or personal care product comprising any of the extrudates and/or beadlets according to the fourth aspect and/or any which were made according to any of the embodiments of the third aspect, and/or which include the composition of matter according to any of the embodiments of the first aspect and/or which were made by any of the methods of the second aspect.

Especially if used in a personal care product, the compositions according to the first aspect, or the products of the fifth aspect which include them, beneficially possess antimicrobial activity. As used herein, the term “antimicrobial activity” or “antimicrobial effect” preferably refers to the capability of inhibiting and/or abolishing the growth of skin microbes, particularly cells of Cutibacterium acnes, Corynebacterium xerosis and/or Malassezia, preferably M. furfur, in a given sample or in a human or animal in need thereof and compared to the inoculum respectively a control as outlined in the examples. The terms “abolishing” and “killing” are used interchangeably herein. The term “growth” in connection with skin microbes and as used herein refers to unwanted or unhealthy growth of the microbes as defined herein, resulting to an increase of microbial cells on the skin compared to a balanced and healthy skin microbiome.

Due to the antimicrobial activity against skin microbes such as Cutibacterium acnes, Corynebacterium xerosis and/or Malassezia, particularly M. furfur, the compositions according to the first aspect and/or the products of the fifth aspect incorporating them may be suitable for the treatment, prevention and/or lessening the symptoms of adverse skin conditions associated with an overpopulation of said microbes, such as acne control application (Cutibacterium acnes), deodorant applications (Corynebacterium xerosis) and hair care such as in particular dandruff applications (Malassezia, preferably M. furfur).

For example, the above-mentioned antimicrobial effect can be used for microbiome balancing, e.g., when applied on an external surface of the human or animal body.

Thus, in another embodiment, the compositions according to the first aspect and/or the products of the fifth aspect which incorporate them may be particularly useful as antimicrobial compounds in cosmetic or pharmaceutical products used for controlling/reducing acne, particularly used to inhibit overpopulation of Cutibacterium acnes on the skin, as components of deodorants, particularly used to inhibit overpopulation of Corynebacterium xerosis causing malodor upon sweating, or for controlling/reducing dandruff, particularly used to inhibit overproduction of Malassezia, preferably M. furfur.

Said antimicrobial effect can, however, also be used to prevent and/or treat diseases linked with a dysbiosis or imbalance of skin microbiota associated with an increased number (or overpopulation)/growth of Cutibacterium acnes, Corynebacterium xerosis and/or Malassezia, particularly M. furfur.

It is well understood that the compositions according to the first aspect and/or the products of the fifth aspect which incorporate them according to the present invention can be used both in the cosmetic sense as well as in the pharmaceutical sense.

A pharmaceutical application is conceivable, for example, for the treatment, prevention and/or lessening of any disorder and disease associated with said microbes such as in particular Cutibacterium acnes and/or Malassezia, preferably M. furfur, in a human (e.g. a patient) or animal in need thereof such as in the treatment, prevention and/or lessening of the symptoms of Pityriasis versicolor, dandruff formation, seborrheic dermatitis, atopic dermatitis, psoriasis and acne.

A cosmetical application is conceivable, if the application is intended to preserve and/or enhance the beauty and/or youthfulness of a person specifically as it relates to the appearance of tissue or skin. Preferred cosmetic (non-therapeutic) applications according to the present invention encompass the treatment, prevention and/or lessening the symptoms of itching skin, the maintenance of skin homeostasis, the provision of microbiome balancing as well as the reduction of malodor formation caused by sweat.

To make use of the antimicrobial activity against Cutibacterium acnes, Corynebacterium xerosis and/or Malassezia, preferably M. furfur, the compositions according to the first aspect and/or the products of the fifth aspect which incorporate them can be used in a multiplicity of applications, such as, for example, cosmetic or pharmaceutical compositions.

Thus, in another embodiment, the compositions according to the present invention are cosmetic or pharmaceutical compositions further comprising a cosmetically or pharmaceutically acceptable carrier. The term “cosmetically or “pharmaceutically acceptable carrier” refers to all carriers and/or excipients and/or diluents conventionally used in cosmetic compositions or pharmaceutical compositions. As used herein, it can be understood to be a physiologically acceptable medium, i.e. a medium compatible for the intended use such as e.g. with keratinous substances, such as the skin, mucous membranes, and keratinous fibers.

The term “cosmetic composition” or “cosmetic product” as used in the present application refers to compositions as defined under the heading “Kosmetika” in Rompp Lexikon Chemie, 10th edition 1997, Georg Thieme Verlag Stuttgart, New York as well as to cosmetic compositions as disclosed in A. Domsch, “Cosmetic Compositions”, Verlag für chemische Industrie (ed. H. Ziolkowsky), 4^{th} edition, 1992 and refers to products/compositions which are used to treat, care for or improve the appearance of the skin and/or scalp.

The term “pharmaceutical composition” or “pharmaceutical products” as used herein refers to compositions which are used for the treatment, prevention and/or lessening of the symptoms of diseases and/or disorders.

Thus, in another advantageous embodiment the cosmetic or pharmaceutical compositions/products according to the present invention are anti-acne compositions/products comprising the compositions according to the first aspect and/or the products of the fifth aspect which incorporate them, which are potentially suitable to kill or significantly inhibit the growth of Cutibacterium acnes. Particularly suitable anti-acne compositions are creams, lotions and the like, preferably in the form of an emulsion such as an O/W emulsion as disclosed herein.

Also advantageous according to the present invention is the use of the compositions according to the first aspect and/or the products of the fifth aspect which incorporate them with all the definitions and preferences as given herein as active compound in anti-dandruff preparations as it has an antimicrobial action against Malassezia, preferably M. furfur, even at very low concentrations of less than about 0.3 wt.-%.

Thus, in another advantageous embodiment the cosmetic or pharmaceutical compositions/products according to the present invention are anti-dandruff compositions comprising said compositions according to the fifth aspect, in an amount of 0.1 to 0.3 wt.-%, based on the total weight of the composition as these compositions are particularly suitable to kill respectively significantly inhibit the growth of Malassezia, preferably M. furfur. Particularly suitable anti-dandruff compositions are shampoos and hair tonics.

The cosmetic or pharmaceutical compositions/products as disclosed herein preferably are aqueous compositions, i.e. compositions which comprise water.

It is furthermore advantageous, that the water content in said cosmetic or pharmaceutical compositions/products according to the present invention is at least about 30 wt.-%, preferably at least about 40, 45, 50, 60, 70, 80, 90 wt.-%, such as e.g. in the range of from 30 to 90 wt.-%, such as from 40 to 90 wt.-%, from 45 to 90 wt.-% or from 50 to 90 wt.-%, based on the total weight of the composition. Further suitable ranges are from 30 to 75 wt.-%, from 30 to 70 wt.-%, from 30 to 60 wt.-% and from 40 to 60 wt.-%.

In a particular embodiment, the products of the fifth aspect are cosmetic compositions intended to be topically applied to mammalian keratinous tissue such as in particular to human/animal skin or the human/animal scalp. Such compositions are also called dermatological compositions. Thus, preferably in certain embodiments of the present invention, the cosmetic compositions are topical cosmetic (i.e. dermatological) compositions/products with all the definitions and preferences as given herein.

The topical cosmetic compositions/products according to the present invention may be leave-on or rinse-off compositions, and include any cosmetic product applied to a human body, primarily for improving appearance, cleansing, odor control or general aesthetics. Preferably the cosmetic compositions of the present invention are leave-on compositions.

It is well understood that the cosmetic compositions/products according to the invention intended for topical application comprise a physiologically acceptable medium, i.e. a medium compatible with keratinous substances, such as the skin, mucous membranes, and keratinous fibers. In particular, the physiologically acceptable medium is a cosmetically acceptable carrier. In embodiments of the present invention, it is preferred that the carrier comprises water.

The term “cosmetically acceptable carrier” (also referred to herein as carrier) refers to all vehicles/carriers conventionally used in cosmetic compositions, i.e. which are suitable for topical application to the keratinous tissue, have good aesthetic properties, are compatible with the actives present in the composition/products, and will not cause any unreasonable safety or toxicity concerns. Such carriers are well-known to one of ordinary skill in the art and can include one or more compatible liquid(s) or solid filler diluent(s), excipient(s), additive(s) or vehicle(s) which are suitable for application to skin.

The exact amount of carrier will depend upon the actual level of the active ingredients and of any other optional ingredients that one of ordinary skill in the art would classify as distinct from the carrier (e.g., other active ingredients).

The cosmetic compositions of the fifth aspect of the present invention preferably comprise from 50% to 99.995%, such as from 50% to 99.95, more preferably from 60% to 99.995%, such as from 60% to 99.95, still more preferably from 75% to 99%, and most preferably, from 80% to 98% such as 90% to 98%, by weight of the composition, of a carrier, based on the total weight of the composition.

The cosmetic compositions/products in accordance with the fifth aspect of the invention can be in the form of a liquid, lotion, a thickened lotion, a gel, a cream, a milk, an ointment, a paste, a powder, a make-up, or a solid tube stick and can be optionally be packaged as an aerosol and can be provided in the form of a mousse such as an aerosol mousse, a foam or a spray foam, a spray, a stick.

Preferably the cosmetic compositions/products according to the fifth aspect of the present invention are in the form of lotions, creams, gels, and tonics. These product forms may be used for a number of applications, including, but not limited to, hand and body lotions, facial moisturizers, anti-ageing preparations, make-ups including foundations, and the like. Any additional components required to formulate such products vary with product type and can be routinely chosen by one skilled in the art.

If the cosmetic compositions of the fifth aspect of the present invention are formulated as an aerosol and applied to the skin as a spray-on product, a propellant is added to the composition.

The cosmetic compositions/products according to the fifth aspect of the present invention can be prepared by conventional methods in the art such as e.g. by admixing the mixture or composition comprising a mixture of cis and trans isomers of retinoids as defined herein, having a cis/trans ratio of less than 0.01 with all the definitions and preferences given herein with the cosmetically acceptable carrier.

The cosmetic composition/products may comprise further ingredients, which may form part of the carrier. Such ingredients are particularly surfactants, emulsifiers, thickeners, and oils. Such suitable surfactants, emulsifiers, thickeners, and oils are well known to a person skilled in the art.

The cosmetic compositions/products of the fifth aspect of the invention (including the carrier) may comprise further conventional (cosmetic) adjuvants and additives, such as preservatives/antioxidants, fatty substances/oils, water, organic solvents, silicones, thickeners, softeners, emulsifiers, antifoaming agents, aesthetic components such as fragrances, surfactants, fillers, anionic, cationic, non-ionic or amphoteric polymers or mixtures thereof, propellants, acidifying or basifying agents, dyes, colorings/colorants, abrasives, absorbents, chelating agents and/or sequestering agents, essential oils, skin sensates, astringents, pigments or any other ingredients usually formulated into such compositions.

If nothing else is stated, the excipients, additives, diluents, etc. mentioned in the following are suitable for the compositions according to the present invention. The necessary amounts of the cosmetic and dermatological adjuvants and additives can, based on the desired product, easily be determined by the skilled person.

The additional ingredients can either be added to the oily phase, the aqueous phase or separately as deemed appropriate. The mode of addition can easily be adapted by a person skilled in the art.

Examples of suitable cosmetic surfactants, emulsifiers, thickeners, oils, excipients, diluents, adjuvants, additives as well as active ingredients commonly used in the skin care industry which are suitable for use in the cosmetic compositions of the present invention are for example described in the International Cosmetic Ingredient Dictionary & Handbook by Personal Care Product Council ([[http://www.]]personalcarecouncil.org[[/]]), accessible by the online INFO BASE ([[http://]]online.personalcarecouncil.org/jsp/Home.jsp), without being limited thereto.

The cosmetically active ingredients useful herein can in some instances provide more than one benefit or operate via more than one mode of action.

Of course, one skilled in this art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

The cosmetic compositions/products according to the present invention are in particular skin care preparations, functional preparations and/or hair care preparations such as most in particularly skin or hair care preparations.

Examples of skin care preparations are, in particular, light protective preparations (sun care preparations), anti-ageing preparations, preparations for the treatment of photo-ageing, body oils, body lotions, body gels, treatment creams, skin protection ointments, moisturizing preparations such as moisturizing gels or moisturizing sprays, face and/or body moisturizers, as well as skin lightening preparations.

Examples of functional preparations are cosmetic compositions containing active ingredients such as hormone preparations, vitamin preparations, vegetable extract preparations, anti-ageing preparations, and/or antimicrobial (antibacterial or antifungal) preparations without being limited thereto.

Examples of hair care preparations which are suitable according to the invention and which may be mentioned are shampoos, hair conditioners (also referred to as hair rinses), hairdressing compositions, hair tonics, hair regenerating compositions, hair lotions, water wave lotions, hair sprays, hair creams, hair gels, hair oils, hair pomades or hair brilliantines. Accordingly, these are always preparations which are applied to the hair and the scalp for a shorter or longer time depending on the actual purpose for which they are used.

Preferably in embodiments of the present invention the skin/functional and/or hair care preparation is a deodorant, an anti-perspirant, an anti-dandruff or an anti-acne composition comprising a composition according to the first aspect of the invention.

In a preferred embodiment, the cosmetic products/compositions according to the invention are in the form of emulsions and/or gels. Even more preferably, the cosmetic compositions are emulsions which contain an oily phase and an aqueous phase such as in particular O/W, W/O, Si/W, W/Si, O/W/O, W/O/W multiple or a pickering emulsions, PIT-emulsions, nano emulsions, micro emulsions, multiple emulsions (e. g. O/W/O- or W/O/W-type).

The amount of the oily phase (i.e. the phase containing all oils and fats including the polar oils) present in such emulsions such as in particular O/W, W/O, Si/W, W/Si, O/W/O, W/O/W multiple or a pickering emulsions is preferably at least about 10 wt.-%, such as in the range from 10 to 60 wt.-%, preferably in the range from 15 to 50 wt.-%, most preferably in the range from 15 to 40 wt.-%, based on the total weight of the composition.

The oil phase according to the invention preferably comprises oils selected from butylenglykoldicaprylat/-dicaprat, dimethicone, propylenglykoldicaprylat/-dicaprat, dicaprylylether, C₁₂₋₁₅-Alkylbenzoat, C₁₈₋₃₈-fatty acid triglyceride, dibutyladipate, cyclomethicone, 2-phenylethylbenzoat, isopropyl lauroyl sarkosinate, caprylic/capric triglyceride as well as mixtures thereof.

The amount of the aqueous phase present in such emulsions is preferably at least about 20 wt.-%, such as in the range from 20 to 90 wt.-%, preferably in the range from 30 to 80 wt.-%, most preferably in the range from 30 to 70 wt.-%, based on the total weight of the composition.

Advantageously in all emulsions of the present invention the ratio of oily phase to aqueous phase is selected in the range of 40:60 to 30:70.

In one particular advantageous embodiment, the cosmetic compositions according to the fifth aspect of the present invention are in the form of an oil-in-water (O/W) emulsion comprising an oily phase dispersed in an aqueous phase in the presence of an O/W emulsifier. The preparation of such O/W emulsions is well known to a person skilled in the art.

If the cosmetic composition according to the fifth aspect of the invention is an O/W emulsion, then it contains advantageously at least one O/W- or Si/W-emulsifier selected from the list of glyceryl stearate citrate, glyceryl stearate SE (self-emulsifying), stearic acid, salts of stearic acid, polyglyceryl-3-methylglycosedistearate. Further suitable emulsifiers are phosphate esters and the salts thereof such as cetyl phosphate (e.g. as Amphisol® A from DSM Nutritional Products Ltd), diethanolamine cetyl phosphate (e.g. as Amphisol® DEA from DSM Nutritional Products Ltd), potassium cetyl phosphate (e.g. as Amphisol® K from DSM Nutritional Products Ltd), sodium cetearylsulfate, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, cetearyl glucoside, lauryl glucoside, decyl glucoside, sodium stearoyl glutamate, sucrose polystearate and hydrated polyisobutene. Furthermore, one or more synthetic polymers may be used as an emulsifier. For example, PVP eicosene copolymer, acrylates/C₁₀₋₃₀ alkyl acrylate crosspolymer, and mixtures thereof.

The at least one O/W, respectively Si/W emulsifier is preferably used in an amount of 0.5 to 10 wt.-%, in particular in the range of 0.5 to 6 wt.-%, such as more in particular in the range of 0.5 to 5 wt.-%, such as most in particular in the range of 1 to 4 wt.-%, based on the total weight of the cosmetic composition.

Particular suitable O/W emulsifiers to be used in the cosmetic compositions according to the fifth aspect encompass phosphate ester emulsifiers such as advantageously 8-10 alkyl ethyl phosphate, C9-15 alkyl phosphate, ceteareth-2 phosphate, ceteareth-5 phosphate, ceteth-8 phosphate, ceteth-10 phosphate, cetyl phosphate, C6-10 pareth-4 phosphate, C12-15 pareth-2 phosphate, C12-15 pareth-3 phosphate, DEA-ceteareth-2 phosphate, DEA-cetyl phosphate, DEA-oleth-3 phosphate, potassium cetyl phosphate, deceth-4 phosphate, deceth-6 phosphate and trilaureth-4 phosphate.

A particular suitable O/W emulsifier to be used in the cosmetic compositions according to the fifth aspect is potassium cetyl phosphate e.g. commercially available as Amphisol® K at DSM Nutritional Products Ltd Kaiseraugst.

Another particular suitable class of O/W emulsifiers are non-ionic self-emulsifying systems derived from olive oil e.g. known as (INCI Name) cetearyl olivate and sorbitan olivate (chemical composition: sorbitan ester and cetearyl ester of olive oil fatty acids) sold under the tradename OLIVEM 1000.

In one particular embodiment of the fifth aspect, the invention relates to cosmetic compositions/products with all the definitions and preferences given herein in the form of O/W emulsions comprising an oily phase dispersed in an aqueous phase in the presence of an O/W emulsifier wherein the O/W emulsifier is potassium cetyl phosphate. The amount of oily phase in such O/W emulsions is preferably at least about 10 wt.-%, more preferably in the range of 10 to 60 wt.-%, most preferably in the range of 15 to 50 wt.-%, such as in the range of 15 to 40 wt.-%, based on the total weight of the composition.

Preferably, the cosmetic compositions according to the fifth aspect of the invention further comprise at least one fatty alcohol (co-emulsifier), such as in particular cetyl alcohol, cetearyl alcohol and/or behenyl alcohol. The total amount of one or several fatty alcohols on the topical compositions according to the invention is preferably selected in the range of about 0.1 to about 10.0 wt.-%, in particular in the range of about 0.5 to 6.0 wt.-% with respect to the total weight of the topical composition.

Preferably, the topical compositions according to the fifth aspect comprise a thickener in particular if the topical composition is in the form of an emulsion to assist in making the consistency of a product suitable. Preferred thickeners are aluminiumsilicates, xanthan gum, hydroxypropylmethylcellulose, hydroxyethylcellulose, polyacrylates such as carbopol (e.g. Carbopol® 980, 981, 1382, 2984, 5984) or mixtures thereof. Further preferred thickeners encompass acrylate/C₁₀₋₃₀ alkyl acrylate copolymers (such as e.g. Pemulen™ TR-1, Pemulen™ TR12, Carbopol® 1328 by NOVEON) as well as Aristoflex® AVC (INCI. Ammonium Acryloyldimethyltaurate/VP Copolymer).

The cosmetic compositions according to the fifth aspect advantageously comprise a preservative. When present, the preservative is preferably used in an amount of 0.1 to 2 wt.-%, more preferably in an amount of 0.5 to 1.5 wt.-%, based on the total weight of the composition.

The cosmetic compositions according to the fifth aspect have a pH in the range of 3 to 10, preferably a pH in the range of 4 to 8, most preferred a pH in the range of pH 4 to 7.5, such as in the range of 5 to 6.5. The pH is adjusted by methods known to a person skilled in the art, e.g. by using an acid such as e.g. citric acid or a base such as e.g. sodium hydroxide (e.g. as aqueous solution), triethanolamine (TEA Care), Tromethamine (Trizma Base) and Aminomethyl Propanol (AMP-Ultra PC 2000), according to standard methods in the art.

The amount of the cosmetic composition to be applied to the skin is not critical and can easily be adjusted by a person skilled in the art. Preferably the amount is selected in the range of 0.1 to 3 mg/cm² skin, such as preferably in the range of 0.1 to 2 mg/cm² skin and most preferably in the range of 0.5 to 2 mg/cm² skin.

Fermentatively-produced retinoid-containing compositions (and the food, feed, pharmaceutical, and/or personal care products into which they are associated) of the current invention can be configured via the selection of components specified above herein, and further readily by following the formulation guidelines herein, as well as by extrapolating from the general approaches taken in the embodiments illustrated in the examples below. The following such examples further illustrate the invention but, of course, should not be construed as in any way limiting its scope.

### EXAMPLES

These examples illustrate embodiments of the instant invention. Table 1 describes a characterization of various retinoid-containing compositions of matter as used in the present examples. Table 2 indicates the results of certain heat-stability performance testing conducted with respect to the examples so characterized in Table 1. Table 3, meanwhile, evidences the bio-based content of an example fermentatively-produced retinoid-containing composition of matter. Finally, Table 4 describes anti-microbial performance testing conducted with respect to other retinoid-containing compositions of matter tuned to have different isomeric ratios.

### General Methods and Strains

All basic molecular biology and DNA manipulation procedures described herein are generally performed according to Sambrook et al. (eds.), Molecular Cloning: A Laboratory Manual. Cold Spring Harbor Laboratory Press: New York (1989) or Ausubel et al. (eds). Current Protocols in Molecular Biology. Wiley: New York (1998). All genetic manipulations exemplified were performed in Yarrowia lipolytica.

Shake plate assay. Typically, 200 μl of 0.075% Yeast extract, 0.25% peptone (0.25× YP) is inoculated with 10 μl of freshly grown Yarrowia and overlaid with 200 μl of Drakeol 5 (Penreco, Karns City, PA, USA) mineral oil, silicone oil, or corn oil with either 2% oleic acid or 2% glucose as a carbon source. Clonal isolates of transformants were grown in 24 well plates (Multitron, 30° C., 800 RPM) in YPD media with one of the overlays indicated earlier for 4 days. The overlay fraction was removed from the shake plate wells and analyzed by HPLC on a normal phase column, with a photo-diode array detector.

DNA transformation. Strains are transformed by overnight growth on YPD plate media 50 μl of cells is scraped from a plate and transformed by incubation in 500 μl with 1 μg transforming DNA, typically linear DNA for integrative transformation, 40% PEG 3550 MW, 100 mM lithium acetate, 50 mM Dithiothreitol, 5 mM Tris-Cl pH 8.0, 0.5 mM EDTA for 60 minutes at 40° C. and plated directly to selective media or in the case of dominant antibiotic marker selection the cells are out grown on YPD liquid media for 4 hours at 30° C. before plating on the selective media. Nourseothricin (Nat) selection was performed on YPD media containing 100 μg/mL nourseothricin and hygromycin (Hyg) selection was performed on YPD containing 100 μg/mL hygromycin. URA3 marker recycling was performed using 5-fluoroorotic acid (FOA). Episomal hygromycin resistance marker (Hyg) plasmids were cured by passage on non-selective media, with identification of Hyg-sensitive colonies by replica plating colonies from non-selective media to hygromycin containing media (100 μg/mL). Hygromycin-resistance markers for integrated hygromycin-marked DNA were recycled using standard methods of cre-recombinase expression and identification of sensitive colonies by replica plating.

Plasmid list. Plasmid, strains, and nucleotide sequences that were used are listed in below and in the sequence listing in Table 5 herein, infra. In general, all non-modified sequences referred to herein are the same as the accession sequence in the database for reference strain CLIB122 (Dujon B, et al, Nature. 2004 Jul. 1; 430(6995):35-44).

### Preparation of Retinoid-Producing Yarrowia Strain and Fermentation Conditions

Strain ML18743, which is described in WO2022090548, was serially transformed with HindIII+XbaI-digested plasmid MB8203 and a HindIII+XbaI-digested plasmid MB9894 (described in the table below), using methods described in WO2022090548, to generate strain ML18743+MB8203+MB9894. This strain was transformed with SfiI-digested plasmid MB9523 to generate new strain ML18934. Strain MHL18934 was then transformed with SfiI-digested plasmid MB7270 to generate new strain ML19284. This strain was made ku70—by CRISPR mutagenesis with plasmid MB9282. To this strain, DNA41 and DNA53 were serially integrated by nuclease-directed integration using homologous recombination to generate new strain ML19649. Strains ML18934, ML19284, and ML19649 was fermented using an ethanol feed using Isopar M as a second phase, as described in WO2022090548 and as further described below and in the sequence listing of Table 5.

| | | | Seq ID No (per |
|---|---|---|---|
| DNA | Insert | Marker | Table 5) |
| MB8203 | Homo sapiens | URA3 | 1 |
| | RDH12 (HsRDH12) | | |
| MB9894 | YALI0F29821 | URA3 | 2 |
| | (YlRDH22) | | |
| MB9523 | LmATF, DrBCO, | URA3 | 3 |
| | FoRDH12 | | |
| MB7270 | CarRP, CarG, CarB | Hyg | 4 |
| MB9282 | Cas9, sgKu70 | Hyg | 5 |
| DNA41 | tHMGR | None | 6 |
| DNA53 | CarB, CarRP, AACS, | None | 7 |
| | MVK | | |

The strains produced as described above were then used in fermentation processes to yield examples 1-4 as described further, below.

### Fermentation

Fermentative product used in Example 1
Production reactor: New Brunswick Bioflo 610.
Seed train: Shakeflask (250 ml) and New Brunswick Bioflo 310
Strain: ML18934
Media: 6.13 g/l Tastone-154, 1.96 g/l MgSO4 7H2O, 0.2 g/l NaCl, 0.33 g/l CaCl 2H2O, 8.18 g/l (NH4)2SO4, 8.47 g/l KH2PO4, 0.2 g/l thiamine, 8.13 g/l DF204 antifoam, trace metals, 50 g/l glucose, 9.86 g/l ethanol and 197.5 g/l Isopar M
Seed media:
   flask: 30 g/l Tastone-154, 22 g/l glucose, 0.5 g/l Basildon antifoam
   bioreactor: 30 g/l Tastone-154, 70 g/l glucose, 10 g/l Basildon antifoam
Batch volume 45 kg (incl inoculum)
Fermentation time: 168 h
Feeds: 6N NH4OH, ethanol (200 proof), DF204 antifoam
Process description: Batch phase ends when initial glucose and ethanol depleted. Ethanol feed fixed rate ramp down to DO of 20%. DO controlled at 20% by agitation, airflow and feed rate. pH controlled at 5.5 with NH4OH.

Fermentative product used in Example 2
Production reactor: New Brunswick Bioflo 610.
Seed train: Shakeflask (250 ml) and New Brunswick Bioflo 310
Strain: ML19284
Media: 1.96 g/l MgSO4 7H2O, 0.2 g/l NaCl, 8 g/l (NH4)2SO4, 16 g/l KH2PO4, 0.8 g/l thiamine, 5 g/l DF204 antifoam, 50 g/l glucose, 9.86 g/l ethanol and 296 g/l Isopar M trace metals: 656 mg/l Citric Acid, 58.8 mg/l FeSO4·7H2O, 6.3 mg/l CuSO4·5H2O, 9.45 mg/l MnSO4·1H2O, 33.2 mg/l ZnSO4·7H2O, 47.3 mg/l CaCl2·2H2O
Seed media:
   flask: 30 g/l Tastone-154, 22 g/l glucose, 0.5 g/l Basildon antifoam
   bioreactor: 30 g/l Tastone-154, 70 g/l glucose, 10 g/l Basildon antifoam
Batch volume 40 kg (incl inoculum)
Fermentation time: 260 h
Feeds: 6N NH4OH, ethanol (200 proof), DF204 antifoam
Process description: Batch phase ends when initial glucose and ethanol depleted. Ethanol feed fixed rate ramp down to DO of 20%. DO controlled at 20% by agitation, airflow and feed rate. pH controlled at 5.5 with NH4OH.

Fermentative product used in Example 3
Production reactor: New Brunswick Bioflo 610.
Seed train: Shakeflask (250 ml) and New Brunswick Bioflo 310
Strain: ML19649
Media: 1.96 g/l MgSO4 7H2O, 0.2 g/l NaCl, 8 g/l (NH4)2SO4, 16 g/l KH2PO4, 0.8 g/l thiamine, 5 g/l DF204 antifoam, 50 g/l glucose, 9.86 g/l ethanol and 296 g/l Isopar M trace metals: 656 mg/l Citric Acid, 58.8 mg/l FeSO4·7H2O, 6.3 mg/l CuSO4·5H2O, 9.45 mg/l MnSO4·1H2O, 33.2 mg/l ZnSO4·7H2O, 47.3 mg/l CaCl2·2H2O
Seed media:
   flask: 30 g/l Tastone-154, 22 g/l glucose, 0.5 g/l Basildon antifoam
   bioreactor: 30 g/l Tastone-154, 70 g/l glucose, 10 g/l Basildon antifoam
Batch volume 40 kg (incl inoculum)
Fermentation time: 260 h
Feeds: 6N NH4OH, ethanol (200 proof), DF204 antifoam
Process description: Batch phase ends when initial glucose and ethanol depleted. Ethanol feed fixed rate ramp down to DO of 20%. DO controlled at 20% by agitation, airflow and feed rate. pH controlled at 5.5 with NH4OH.

Fermentative product used in Example 4
Production reactor: New Brunswick Bioflo 610.
Seed train: Shakeflask (250 ml) and New Brunswick Bioflo 310
Strain: ML18934
Media: 6.13 g/l Tastone-154, 1.96 g/l MgSO4 7H2O, 0.2 g/l NaCl, 0.33 g/l CaCl 2H2O, 8.18 g/l (NH4)2SO4, 8.47 g/l KH2PO4, 0.2 g/l thiamine, 8.13 g/l DF204 antifoam, trace metals, 50 g/l glucose, 9.86 g/l ethanol and 197.5 g/l Isopar M
Seed media:
   flask: 30 g/l Tastone-154, 22 g/l glucose, 0.5 g/l Basildon antifoam
   bioreactor: 30 g/l Tastone-154, 70 g/l glucose, 10 g/l Basildon antifoam
Batch volume 45 kg (incl inoculum)
Fermentation time: 168 h
Feeds: 6N NH4OH, ethanol (200 proof), DF304 antifoam
Process description: Batch phase ends when initial glucose and ethanol depleted. Ethanol feed fixed rate ramp down to DO of 20%. DO controlled at 20% by agitation, airflow and feed rate. pH controlled at 5.5 with NH4OH.
Comment: Fermentations tested high dco2 concentrations. Contamination detected.

### DSP & Creation of Dry Crystals

Each of examples 1-4 was prepared into dry crystalline form for subsequent characterization and performance testing via the following process. First, each fermentative product described above (each containing retinyl acetate as retinoid component) was provided in a concentration of light phase from centrifugation in a lab scale short path evaporator (0.03 m2) at ca. 1 mbar absolute pressure and 80° C. jacket temperature to reach a retinyl acetate fraction in the residue of approx. 40-45 wt %. Next, the concentrated residue was mixed with water-free ethanol to give a solution at 20° C. containing 45-47 wt % of ethanol. This solution was then cooled down in a 250 ml double-jacketed reactor to 14° C. under stirring, at which time seed crystals were added and the temperature was maintained at 14° C. for one additional hour. The reactor was then cooled down with a cooling ramp of 5° C./h to −20° C. and held for approximately 16 hours at −20° C. The resulting suspension was then filtered over a Buchner vacuum funnel, pre-cooled at −20° C. and washed twice with cold ethanol (−20° C.) to obtain wet crystals. These crystals were finally dried in a vacuum drying oven at room temperature and reduced pressure to remove any residual ethanol to yield the dry crystals.

### Preparation of Example 5

Sample 5 was provided as dry retinyl acetate crystals produced via chemical synthesis, as is readily available from DSM Nutritional Products AG, Kaiseraugst Switzerland. It is used herein as a control which is representative of non-fermentatively produced retinoid-containing compositions.

### Preparation of Example 6

Example 6 was composition of matter in crystalline form composed predominantly (i.e. >80 wt. %) of a retinyl acetate and its fermentative residue thereof which was fermentatively produced generally according to the same techniques and methods used with regards to the preparation of each of examples 1-4, supra. Notably, the same ethanol used as carbon source in the fermentation of each of examples 1-4 was used during the fermentation process which led to the production of example 6 as well.

Because of the fact that the same carbon source was used during fermentation, it can be concluded that the bio-based profile of example 6 is representative of those of examples 1-4 as well.

### Preparation of Examples 7-11

The compositions utilized in examples 7-11 were prepared according to standard techniques in the art to which this invention applies, and further as described below. Retinoid mixtures comprising retinyl acetate as defined herein can be produced as described in WO2022090549 (Ex. 1). The samples were controlled to the extent possible to assure that only the cis/trans isomer ratio of the retinoid component for the respective sample series (i.e. 7-9 and 10-11) would vary. The cis/trans ratio and the specific nature of the retinoid component for each sample is indicated in Table 4 below.

### Sample Characterization

Retinoid quantification: Analysis of retinoids were carried out with a C4 reverse phase retinoid method (see below). The addition of all added intermediates gives the total amount of retinoids.

C4 reverse phase chromatography. For exact determination of discrete retinoids, the long run reverse phase system was used. Analytes were separated using the Agilent 1290 instrument with YMC Pro C4, 150×3.0 mm, 3 um column (YMC America, Allentown PA) stationary phase, and a 5 μl injection loop volume and column and sample tray controlled at 23° C. with gradients described in Table B. Analytes were detected at 210, 230 nm and 325 nm and the peak's identity was verified with LCMS. The analytes were separated as discrete peaks that were assigned according to the table below.

TABLE A
list of analytes using C4-reverse phase method. The
addition of all added intermediates gives the total
amount retinoids. “RT” means retention time.

| Compound | RT (min) | λ max (nm) |
|---|---|---|
| Dihydro-actinidiolide | 12.99 | 210 |
| Farnesol | 17.90 | 210 |
| Z-Retinol | 20.21 | 325 |
| E-Retinol | 20.32 | 325 |
| Dihydro Retinol | 20.75 | 290 |
| Z-Retinal | 20.89 | 380 |
| E-Retinal | 21.02 | 380 |
| t-Retinyl Acetate | 22.15 | 325 |
| Cis Retinyl Acetate | 22.35 | 325 |
| Dihydro Retinyl Acetate | 22.6 | 290 |
| Retinyl palmitate | 27.8 | 325 |
| β-carotene | 28.9 | 325 |
| Retinyl oleate | 29.3 | 325 |

TABLE B
Method Gradient with solvent
Solvent A:
Solvent B: Acetonitrile
Solvent C: Water
Solvent D: Water/Acetonitrile/Methanesulfonic acid 1000/25/1

| Time | | | | | Flow | Pressure |
|---|---|---|---|---|---|---|
| (min) | % A | % B | % C | % D | (ml/min) | (psi/bar) |
| 0 | | 5 | 85 | 10 | 0.5 | 200 |
| 20 | | 98 | 0 | 2 | 0.5 | |
| 35 | | 98 | 0 | 2 | 0.5 | |
| 35.1 | | 5 | 85 | 10 | 0.5 | |
| 40 | | 5 | 85 | 10 | 0.5 | |

Method Calibration. Method is calibrated using high purity retinyl acetate received from DSM Nutritional Products, Kaiseraugst, CH. Retinols and retinal are quantitated against retinyl acetate. Dilutions described in Table 3 are prepared as follows. 40 mg of retinyl acetate is weighed into a 100 mL volumetric flask, and dissolved in ethanol, yielding a 400 μg/mL solution. This solution is sonicated as required to ensure dissolution. 5 mL of this 400 μg/mL solution is diluted into 50 mL (1/10 dilution, final concentration 40 μg/mL), 5 mL into 100 mL (1/20 dilution, final concentration 20 ug/mL), 5 mL of 40 μg/mL into 50 mL (1/10 dilution, final concentration 4 μg/mL), 5 mL of 20 μg/mL into 50 mL (1/10 dilution, 2 μg/mL), using 50/50 methanol/methyl tert-butyl ether (MTBE) as the diluent. All dilutions are done in volumetric flasks. Purity of retinyl acetate is determined by further diluting the 400 μg/mL stock solution 100-fold (using a 2 mL volumetric pipet and a 200 mL volumetric flask) in ethanol. Absorbance of this solution at 325 nm using ethanol is taken as the blank, with adjustment of the initial concentration using the equation (Abs*dilution (100)*molecular weight (328.5)/51180=concentration in mg/mL). Because of quick out-maximization of UV absorbance of retinyl acetate, lower concentrations are better.

TABLE C

| Stock, Dilution | Final Concentration (μg/mL) |
|---|---|
| 20 μg/mL, 1/10 | 2 |
| 40 μg/mL, 1/10 | 4 |
| 400 μg/mL, 1/20 | 20 |
| 400 μg/mL, 1/10 | 40 |
| Stock soln., 1/2 | 400 |

Sample preparation. 7 mg of accurately weighed crystals is dissolved in 10 mL tetrahydrofuran (THF) prior injection.

Isopar M quantification: Analysis of IsoparM were carried out with GC-FID method (see below).

For determination of IsoparM we used an Agilent 7890 instrument or similar with a HP-5 Agilent column with length 30 m, 0.25 mm internal diameter and 0.25 um film thickness. H2 was used as carrier gas at a flow rate of 1.7 mL/min. 1 ul volume sample was injected at an inlet temperature of 250° C. with a split ratio to FID detector of 50:1. Detector temperature was set at 325° C. with a H2 flow of 40 mL/min and air flow of 250 mL/min. The oven temperature program is depicted at table D.

TABLE D
GC oven temperature program

| | Ramp rate | Hold | Hold time | Total ramp time |
|---|---|---|---|---|
| No | [° C./min] | temperature [° C.] | [min] | [min] |
| | | 40 | 3 | 3.000 |
| 1 | 15 | 180 | 0 | 12.333 |
| 2 | 30 | 280 | 5 | 20.667 |
| 3 | | | | 25.667 |

### Method Calibration

n-Decane was used as internal standard (IS). Internal standard solution was prepared by diluting 500 uL of n-Decane with isopropyl alcohol (IPA, HPLC grade) in a 50 mL volumetric flask. The IsoparM standard solution was prepared by diluting 25 uL of accurately weighed IsoparM (Exxon Mobile) in 1 mL of the internal standard solution.

Isopar M was quantified with respect to a single point response factor of n-Decane according to formula described below:

$$Rf = \frac{{Area}_{IS}}{{Area}_{IsoparM}} \times \frac{{Weight}_{IsoparM}}{{Weight}_{IS}}$$

Area_{IS}=area of n-Decane peak in calibration run
Area_{IsoparM}=area of Isopar M in calibration run
Weight_{IS}=weight of n-Decane in calibration run
Weight_{IsoparM}=weight of Isopar M in calibration run
   The retention times of IsoparM, internal standard and IPA is listed in table E.

TABLE E
GC-FID retention time of IsoparM and n-Decane

Amount of Isopar M was calculated with:
$IsoparM ( \% ) = \frac{{Area}_{IsoparM}}{{Area}_{IS}} \times \frac{{Weight}_{IS}}{{Weight}_{IsoparM}} \times Rf \times 100$
Where:
Area_{IsoparM} = area of IsoparM in sample analysis
Area_{IS} = area of internal standard peak in sample analysis
Weight_{ISTD} = weight of internal standard in sample preparation (mg)
Weight_{IsoparM} = sample weight (mg)
The result is present as percentage (%) of Isopar M in product.

Sample preparation. 30 mg of accurately weighed crystals is dissolved in 10 mL of internal standard solution injection.

The values measured via the method described above are reported in Table 1, below. Below the row labelled by “Total” in Table 1 below, there are three additional calculated values. “Cis-isomer %” represents the total quantity of cis-isomers (in this case the row labelled with “cis-retinyl acetate”) relative to the total weight of the retinoid component (in this instance the row labelled with “Retinyl acetate (all species)”) (the “Cis-isomer %” equals the value in the row labelled “cis-retinyl acetate” divided by the value in the row labelled “Retinyl acetate (all species)”, expressed as a % (i.e. multiplied by 100) and rounded to the nearest hundredth of a percent). “FARE” is determined adding the quantity of retinyl palmitate and retinyl oleate detected (no other fatty acid retinyl esters were detected) in the composition. Finally, “WSFRV” represents weighted select fermentation residue value, which was determined by adding the value derived for “Cis-isomer %” content with 10% of the determined “FARE” content.

TABLE 1
Chemical characterization
All amounts are in % wt. relative to the
entire composition unless otherwise stated.

| Example | 1 | 2 | 3 | 4 | 5 |
|---|---|---|---|---|---|
| Ethanol | 0.22% | 0.41% | 1.93% | 0.70% | 0.00% |
| Dihydro- | 0.04% | 0.00% | 0.00% | 0.00% | 0.00% |
| actinidiole | | | | | |
| Z-retinol | 0.29% | 0.04% | 0.37% | 0.35% | 0.02% |
| E-retinol | 1.01% | 0.32% | 2.02% | 1.28% | 0.01% |
| Dihydro- | 0.08% | 0.03% | 0.08% | 0.00% | 0.02% |
| retinol | | | | | |
| Z-retinal | 0.02% | 0.04% | 0.06% | 0.08% | 0.00% |
| E-retinal | 0.03% | 0.16% | 0.08% | 0.06% | 0.00% |
| cis-retinyl | 0.69% | 0.39% | 0.70% | 0.90% | 2.64% |
| acetate | | | | | |
| Retinyl | 90.53% | 89.27% | 87.63% | 93.28% | 97.14% |
| acetate (all | | | | | |
| species) | | | | | |
| Dihydro- | 0.04% | 0.04% | 0.05% | 0.06% | 0.00% |
| retinyl acetate | | | | | |
| Isopar M | 1.07% | 1.04% | 0.74% | 1.85% | 0.00% |
| Farnesol | 0.0% | 0.0% | 0.0% | 0.0% | No data |
| Retinyl | 0.00% | 1.70% | 0.56% | 0.19% | 0.00% |
| palmitate | | | | | |
| Retinyl oleate | 0.20% | 2.14% | 1.01% | 0.30% | 0.00% |
| β-carotene | 0.00% | 1.47% | 0.99% | 0.22% | 0.00% |
| Total | 94.22% | 95.58% | 95.23% | 99.05% | 99.83% |
| Cis-isomer % | 0.76% | 0.44% | 0.80% | 0.96% | 2.72% |
| FARE | 0.20% | 3.84% | 1.57% | 0.49% | 0.00% |
| WSFRV | 0.78 | 0.82 | 0.96 | 1.01 | 2.72 |

### Oxidative Stability Thermal Stress Test

To determine oxidative stability of examples 1-5, each sample, the preparation of which is described above, was placed under ambient conditions (20° C. and 50% relative humidity preferred) in its own small brown glass vial, blanketed with argon, and put into an oven preheated to 105° C. Thereafter, the samples remained in the oven for 3 hours. After three hours the samples were taken out of the oven and analyzed via the same method(s) utilized to determine “initial” values as reported in Table 1 above. The values for each component were measured after the thermal stress test and compared to the values determined prior to the test. The relative difference from the second measurement to the first was then calculated, with the values reported in Table 2 below. For clarity, example 1 indicated a total retinoid component (see row in Table 1 above labelled “all retinyl acetate (all species)”) prior to the thermal stress test of 90.53%; the change indicated in Table 2 of −1.31% below signifies that the total retinoid component determined after the thermal stress test was 89.22% (by weight relative to the entire sample weight).

TABLE 2
Heat Stability Performance
Specified in % change unless otherwise specified

| Example | 1 | 2 | 3 | 4 | 5 |
|---|---|---|---|---|---|
| Ethanol | −0.17% | 0.14% | 0.25% | 0.47% | 0.00% |
| Dihydro- | 0.04% | −0.03% | −0.04% | 0.00% | −0.01% |
| actinidiole | | | | | |
| Z-retinol | 0.06% | 0.01% | 0.03% | 0.03% | −0.04% |
| E-retinol | 0.14% | 0.04% | 0.25% | 0.13% | −0.01% |
| Dihydro- | 0.04% | −0.01% | 0.00% | −0.05% | 0.00% |
| retinol | | | | | |
| 9Z-retinal | −0.01% | −0.01% | −0.01% | 0.04% | 0.00% |
| E-Retinal | 0.00% | 0.03% | 0.01% | −0.02% | −0.01% |
| cis-retinyl | −0.75% | −0.44% | −0.64% | −1.11% | −0.22% |
| acetate | | | | | |
| Retinyl | −1.31% | −4.10% | −4.86% | −5.00% | −5.50% |
| acetate (all | | | | | |
| species) | | | | | |

### Bio-Based Content

In order to determine bio-based content, samples were sent to Isolab GmbH Laboratorium fur Stabil-Isotopenanalytik in Schweitenkirchen, Germany. The samples were analyzed for ¹⁴C isotope content using a liquid scintillation counting method. This lab also utilizes the QMA-M-03 method to determine stable isotope ratios ¹³C, ¹⁵N, ³⁴S (using EA-IRMS), ¹⁸O (using equilibration-IRMS) and ²H (using HTC-IRMS) of plant-based food products and their ingredients. The amount of ¹⁴C content was then calculated in order to determine the relative quantity of carbon atoms present in the sample that were of recent natural materials (i.e. did not include carbon atoms from fossil-based sources such as petroleum). The results are reported as “14C Activity”, expressed in terms of %. Such results of this bio-based test for example 6 is depicted in Table 3 below:

TABLE 3
Bio-based content
Specified in % unless otherwise specified

| Sample | ¹⁴C Activity |
|---|---|
| Example 6 | 102.2% ± 3.7% |

### Antimicrobial Efficacy Test

The antimicrobial efficacy of retinol and retinyl acetate having different cis/trans ratio has been assessed in analogy to the regulatory challenge test method (NF EN ISO11930). Thus, solutions of the respective retinol and retinyl acetate mixtures (reference samples and inventive mixtures with a purity of about 98%) were prepared in physiological serum with 0.85 wt.-% NaCl under sterile conditions. The presence of cis and trans-isomers in the retinoid component were calculated by reversed phase C4 HPLC analysis, with the total cis-isomer content in the retinoid component, relative to the total weight of trans-isomers in the retinoid component detected, is reported in Table 4 below along the row labelled “cis/trans ratio”. Also reported for each sample was the specific retinoid used as the retinoid component (either retinyl acetate or retinol for all samples).

The solutions of the active(s) were then deposed in 96-deep well plates (1.6 ml/well) at a concentration as specified by wt. % in Table 4 below. The wells were contaminated with Cutibacterium acnes, Corynebacterium xerosis, and Malassezia furfur, respectively, to obtain the initial contamination. The initial contamination count, which is listed as “initial inoculum count” for each test as indicated in Tale 4 below, was determined to be 100,000. After contamination, each well was thoroughly mixed to ensure a homogeneous distribution of the microbes. Then each plate was incubated at 22° C. for 24 h. The counting of the (remaining) population was carried out 24 hours after contamination, as indicated below in Table 4 by the rows labelled “24 hr”. The change in count from the initial inoculum count to the 24-hour count (in terms of percentage) is reflected in the Table below by the rows labelled “A R”. The results are shown in Table 4 below.

TABLE 4
Anti-microbial efficacy

| | Ex. 7 | Ex. 8 | Ex. 9 | Ex. 10 | Ex. 11 |
|---|---|---|---|---|---|
| Retinoid component | Retinyl | Retinyl | Retinyl | Retinol | Retinol |
| | acetate | acetate | acetate | | |
| Cis/Trans Ratio (%) | 3% | 0.49% | 0.25% | 1% | 0.3% |

Cutibacterium acnes - initial inoculum count: 100,000

| | | | | | |
|---|---|---|---|---|---|
| Concentration | 0.1% | 0.1% | 0.1% | 0.25% | 0.25% |
| 24 hr | 370,000 | 70,000 | 70,000 | 1,000 | 700 |
| Δ R | 270% | −30% | −30% | −99% | −99.3% |

Corynebacterium xerosis - initial inoculum count: 100,000

| | | | | | |
|---|---|---|---|---|---|
| Concentration | 0.1% | 0.1% | 0.1% | No data | No data |
| 24 hr | 370,000 | 70,000 | 70,000 | No data | No data |
| Δ R | 270% | −30% | −30% | No data | No data |

Corynebacterium xerosis - initial inoculum count: 100,000

| | | | | | |
|---|---|---|---|---|---|
| Concentration | 0.25% | 0.25% | 0.25% | No data | No data |
| 24 hr | 40,000 | 37,000 | 10,000 | No data | No data |
| Δ R | −60% | −63% | −90% | No data | No data |

Malassezia furfur - initial inoculum count: 100,000

| | | | | | |
|---|---|---|---|---|---|
| Concentration | No data | No data | No data | 0.25% | 0.25% |
| 24 hr | No data | No data | No data | 100,000 | 40,000 |
| Δ R | No data | No data | No data | 0% | −60% |

### Discussion of Results

From Table 2, it is shown that a variety of fermentatively-produced retinoid-containing compositions (i.e. examples 1-4) were demonstrated to possess comparable or superior oxidative stability of the retinoid component (retinyl acetate in each of such examples) when compared to a chemically-synthesized analogue, as indicated by the relative loss of retinyl acetate throughout the duration of the heat stability test. Specifically, while the control (example 5) exhibited a 5.50% reduction in retinyl acetate, examples 2-4 performed similarly or slightly better, exhibiting a reduction between 4.10-5.00%. Example 1 performed the best, losing only 1.31% of retinyl acetate content after being subjected to the heat stability test. This result is particularly surprising and unexpected, given the compositions of examples 1-4 each contain one or more fermentative residue constituents that were expected to contribute detrimentally to such composition's oxidative stability.

As can be seen in Table 3, the carbon-14 activity of the composition of matter indicated by example 6 suggests that—within the margin of error of the test—no proportion of fossil carbon could be detected. Therefore, according to this test, such sample can be considered to be completely “bio-based”. This result can be similarly applied to each of examples 1-4 as well, which were prepared via similar methods and using an identical carbon source during the fermentation process.

As can be seen in Table 4, the specific cis/trans mixture of retinyl acetate and retinol, respectively, according to the present invention has an excellent effect in anti-acne application, i.e. against growth of Cutibacterium acnes. A retinyl acetate mixture with a cis isomer content of 0.49% (Ex. 8) and 0.25% (Ex. 9) was able to reduce Cutibacterium acnes by about 30%, whereas a reference having a cis isomer content of 3% allowed for a 270% growth in the bacterium. A similar effect was observed in retinol-containing compositions as well, with a retinol composition (Ex. 11) having a cis isomer content of 0.3% reducing the bacterium by 99.3%, vs. only a 99.0% reduction in an analogous composition having a cis isomer content of 1%. The efficacy of commercially available trans-retinol and trans-retinyl acetate, respectively (e.g. Dry Vitamin A-Acetate 500 or Retinol 50 C from BASF or Microvit® A from Adisseo) against growth of Cutibacterium acnes are in the same range as the reference samples (not shown).

Similar trends can be observed with regards to activity of Corynebacterium xerosis and Malessezia furur from Table 4. Notably, an increase in concentration of the retinoid-containing composition had the effect of further improving antimicrobial efficacy as observed in each of examples 7, 8, and 9 when tested against Corynebacterium xerosis.

Finally, certain compositions with retinol as the retinoid component are demonstrated to have increasing effectiveness against the dandruff-producing bacterium Malessezia furfur. Specifically, it was demonstrated that a composition having a cis isomer content of 0.1% reduced activity by 60%, whereas a composition having a cis isomer content of 1% neither reduced nor allowed growth of the same bacterium.

### Additional Exemplary Embodiments

See claims.

Unless otherwise specified, the term wt. % means the amount by mass of a particular constituent relative to the entire liquid radiation curable composition into which it is incorporated.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While the invention has been described in detail and with reference to specific embodiments thereof, it will be apparent to one of ordinary skill in the art that various changes and modifications can be made therein without departing from the spirit and scope of the claimed invention.

TABLE 5
Sequence Listing

| | | SEQ. |
|---|---|---|
| | | ID |
| Name | Sequence 5′ to 3′ | NO: |
| MB8 | AAGCGGAAGAGCGCCCAATACGCAAACCGCCTCTCCCCGC | 1 |
| 203 | GCGTTGGCCGATTCATTAATGCAGCTGGCACGACAGGTTT | |
| | CCCGACTGGAAAGCGGGCAGTGAGCGCAACGCAATTAATG | |
| | TGAGTTAGCTCACTCATTAGGCACCCCAGGCTTTACACTT | |
| | TATGCTTCCGGCTCGTATGTTGTGTGGAATTGTGAGCGGA | |
| | TAACAATTTCACACAGGAAACAGCTATGACCATGATTACG | |
| | CCAAGCTTGGTACCAGAGACGGGTTGGCGGCGTATTTGTG | |
| | TCCCAAAAAACAGCCCCAATTGCCCCAATTGACCCCAAAT | |
| | TGACCCAGTAGCGGGCCCAACCCCGGCGAGAGCCCCCTTC | |
| | ACCCCACATATCAAACCTCCCCCGGTTCCCACACTTGCCG | |
| | TTAAGGGCGTAGGGTACTGCAGTCTGGAATCTACGCTTGT | |
| | TCAGACTTTGTACTAGTTTCTTTGTCTGGCCATCCGGGTA | |
| | ACCCATGCCGGACGCAAAATAGACTACTGAAAATTTTTTT | |
| | GCTTTGTGGTTGGGACTTTAGCCAAGGGTATAAAAGACCA | |
| | CCGTCCCCGAATTACCTTTCCTCTTCTTTTCTCTCTCTCC | |
| | TTGTCAACTCACACCCGAAATCGTTAAGCATTTCCTTCTG | |
| | AGTATAAGAATCATTCGCTAGCCACAAAAATGATGGCTCC | |
| | CTCCATTCGAAAGTTCTTTGCTGGTGGTGTGTGTCGAACT | |
| | AACGTTCAGCTTCCCGGTAAGGTGGTTGTCATCACTGGTG | |
| | CCAACACCGGCATTGGCAAGGAGACTGCCCGAGAGCTCGC | |
| | TTCCCGAGGAGCCCGAGTTTACATTGCTTGCCGAGATGTT | |
| | CTGAAGGGCGAGTCTGCTGCCTCTGAGATTCGAGTTGACA | |
| | CTAAGAACTCCCAGGTGCTCGTGCGAAAGCTCGACCTTTC | |
| | CGACACTAAGTCTATCCGAGCCTTTGCTGAGGGCTTTCTC | |
| | GCTGAGGAGAAGCAGCTTCACATTCTGATTAACAACGCTG | |
| | GAGTTATGATGTGTCCTTACTCTAAGACTGCTGATGGTTT | |
| | CGAGACTCACCTCGGAGTCAACCACCTGGGCCACTTCCTC | |
| | CTCACCTACCTGCTCCTGGAGCGACTCAAGGTGTCTGCCC | |
| | CTGCCCGAGTGGTTAACGTTTCCTCCGTGGCTCACCACAT | |
| | TGGCAAGATTCCCTTCCACGACCTCCAGTCCGAGAAGCGA | |
| | TACTCCCGAGGTTTTGCTTACTGCCACTCCAAGCTGGCCA | |
| | ACGTTCTCTTTACCCGAGAGCTGGCCAAGCGACTCCAGGG | |
| | AACCGGCGTCACCACCTACGCCGTTCACCCCGGTGTCGTC | |
| | CGATCCGAGCTGGTCCGACACTCCTCCCTGCTCTGCCTGC | |
| | TCTGGCGACTCTTCTCCCCCTTCGTTAAGACCGCCCGAGA | |
| | GGGTGCCCAGACCTCCCTGCACTGCGCCCTGGCTGAGGGC | |
| | CTGGAGCCCCTGTCTGGCAAGTACTTCTCTGACTGCAAGC | |
| | GAACCTGGGTGTCTCCCCGAGCTCGAAACAACAAGACTGC | |
| | CGAGCGACTCTGGAACGTTTCCTGTGAGCTTCTCGGTATT | |
| | CGATGGGAGTAGACGCGTGCAATTAACAGATAGTTTGCCG | |
| | GTGATAATTCTCTTAACCTCCCACACTCCTTTGACATAAC | |
| | GATTTATGTAACGAAACTGAAATTTGACCAGATATTGTTG | |
| | TAAATAGAAAATCTGGCTTGTAGGTGGCAAACTAGTGGTG | |
| | TGTTCTGTGGAGCATTCTCACTTTTGGTAAACGACATTGC | |
| | TTCAAGTGCAGCGGAATCAAAAAGTATAAAGTGGGCAGCG | |
| | AGTATACCTGTACAGACTGTAGGCGATAACTCAATCCAAT | |
| | TACCCCCCACAACATGACTGGCCAAACTGATCTCAAGACT | |
| | TTATTGAAATCAGCAACACCGATTCTCAATGAAGGCACAT | |
| | ACTTCTTCTGCAACATTCACTTGACGCCTAAAGTTGGTGA | |
| | GAAATGGACCGACAAGACATATTCTGCTATCCACGGACTG | |
| | TTGCCTGTGTCGGTGGCTACAATACGTGAGTCAGAAGGGC | |
| | TGACGGTGGTGGTTCCCAAGGAAAAGGTCGACGAGTATCT | |
| | GTCTGACTCGTCATTGCCGCCTTTGGAGTACGACTCCAAC | |
| | TATGAGTGTGCTTGGATCACTTTGACGATACATTCTTCGT | |
| | TGGAGGCTGTGGGTCTGACAGCTGCGTTTTCGGCGCGGTT | |
| | GGCCGACAACAATATCAGCTGCAACGTCATTGCTGGCTTT | |
| | CATCATGATCACATTTTTGTCGGCAAAGGCGACGCCCAGA | |
| | GAGCCATTGACGTTCTTTCTAATTTGGACCGATAGCCGTA | |
| | TAGTCCAGTCTATCTATAAGTTCAACTAACTCGTAACTAT | |
| | TACCATAACATATACTTCACTGCCCCAGATAAGGTTCCGA | |
| | TAAAAAGTTCTGCAGACTAAATTTATTTCAGTCTCCTCTT | |
| | CACCACCAAAATGCCCTCCTACGAAGCTCGAGCTAACGTC | |
| | CACAAGTCCGCCTTTGCCGCTCGAGTGCTCAAGCTCGTGG | |
| | CAGCCAAGAAAACCAACCTGTGTGCTTCTCTGGATGTTAC | |
| | CACCACCAAGGAGCTCATTGAGCTTGCCGATAAGGTCGGA | |
| | CCTTATGTGTGCATGATCAAGACCCATATCGACATCATTG | |
| | ACGACTTCACCTACGCCGGCACTGTGCTCCCCCTCAAGGA | |
| | ACTTGCTCTTAAGCACGGTTTCTTCCTGTTCGAGGACAGA | |
| | AAGTTCGCAGATATTGGCAACACTGTCAAGCACCAGTACA | |
| | AGAACGGTGTCTACCGAATCGCCGAGTGGTCCGATATCAC | |
| | CAACGCCCACGGTGTACCCGGAACCGGAATCATTGCTGGC | |
| | CTGCGAGCTGGTGCCGAGGAAACTGTCTCTGAACAGAAGA | |
| | AGGAGGACGTCTCTGACTACGAGAACTCCCAGTACAAGGA | |
| | GTTCCTGGTCCCCTCTCCCAACGAGAAGCTGGCCAGAGGT | |
| | CTGCTCATGCTGGCCGAGCTGTCTTGCAAGGGCTCTCTGG | |
| | CCACTGGCGAGTACTCCAAGCAGACCATTGAGCTTGCCCG | |
| | ATCCGACCCCGAGTTTGTGGTTGGCTTCATTGCCCAGAAC | |
| | CGACCTAAGGGCGACTCTGAGGACTGGCTTATTCTGACCC | |
| | CCGGGGTGGGTCTTGACGACAAGGGAGACGCTCTCGGACA | |
| | GCAGTACCGAACTGTTGAGGATGTCATGTCTACCGGAACG | |
| | GATATCATAATTGTCGGCCGAGGTCTGTACGGCCAGAACC | |
| | GAGATCCTATTGAGGAGGCCAAGCGATACCAGAAGGCTGG | |
| | CTGGGAGGCTTACCAGAAGATTAACTGTTAGAGGTTAGAC | |
| | TATGGATATGTAATTTAACTGTGTATATAGAGAGCGTGCA | |
| | AGTATGGAGCGCTTGTTCAGCTTGTATGATGGTCAGACGA | |
| | CCTGTCTGATCGAGTATGTATGATACTGCACAACCTGTGT | |
| | ATCCGCATGATCTGTCCAATGGGGCATGTTGTTGTGTTTC | |
| | TCGATACGGAGATGCTGGGTACAAGTAGCTAATACGATTG | |
| | AACTACTTATACTTATATGAGGCTTGAAGAAAGCTGACTT | |
| | GTGTATGACTTATTCTCAACTACATCCCCAGTCACAATAC | |
| | CACCACTGCACTACCACTACACCTCGAGCATGCATCTAGT | |
| | GGTGTGTTCTGTGGAGCATTCTCACTTTTGGTAAACGACA | |
| | TTGCTTCAAGTGCAGCGGAATCAAAAAGTATAAAGTGGGC | |
| | AGCGAGTATACCTGTACAGACTGTAGGCGATAACTCAATC | |
| | CAATTACCCCCCACAACATGACTGGCCAAACTGATCTCAA | |
| | GACTTTATTGAAATCAGCAACACCGATTCTCAATGAAGGC | |
| | ACATACTTCTTCTGCAACATTCACTTGACGCCTAAAGTTG | |
| | GTGAGAAATGGACCGACAAGACATATTCTGCTATCCACGG | |
| | ACTGTTGCCTGTGTCGGTGGCTACAATACGTGAGTCAGAA | |
| | GGGCTGACGGTGGTGGTTCCCAAGGAAAAGGTCGACGAGT | |
| | ATCTGTCTGACTCGTCATTGCCGCCTTTGGAGTACGACTC | |
| | CAACTATGAGTGTGCTTGGATCACTTTGACGATACATTCT | |
| | TCGTTGGAGGCTGTGGGTCTGACAGCTGCGTTTTCGGCGC | |
| | GGTTGGCCGACAACAATATCAGCTGCAACGTCATTGCTGG | |
| | CTTTCATCATGATCACATTTTTGTCGGCAAAGGCGACGCC | |
| | CAGAGAGCCATTGACGTTCTTTCTAATTTGGACCGATAGC | |
| | CGTATAGTCCAGTCTATCTATAAGTTCAACTAACTCGTAA | |
| | CTATTACCATAACATATACTTCACTGCCCCAGATAAGGTT | |
| | CCGATAAAAAGTTCTGCAGACTAAATTTATTTCAGTCTCC | |
| | TCTTCACCACCAAAATGCCCTCCTACGAAGCTCGAGCTAA | |
| | CGTCCACAAGTCCGCCTTTGCCGCTCGAGTGCTCAAGCTC | |
| | GTGGCAGCCAAGAAAACCAACCTGTGTGCTTCTCTGGATG | |
| | TTACCACCACCAAGGGGCCGCTCGAGCATGCATCTAGAGG | |
| | GCCCAATTCGCCCTATAGTGAGTCGTATTACAATTCACTG | |
| | GCCGTCGTTTTACAACGTCGTGACTGGGAAAACCCTGGCG | |
| | TTACCCAACTTAATCGCCTTGCAGCACATCCCCCTTTCGC | |
| | CAGCTGGCGTAATAGCGAAGAGGCCCGCACCGATCGCCCT | |
| | TCCCAACAGTTGCGCAGCCTGAATGGCGAATGGACGCGCC | |
| | CTGTAGCGGCGCATTAAGCGCGGCGGGTGTGGTGGTTACG | |
| | CGCAGCGTGACCGCTACACTTGCCAGCGCCCTAGCGCCCG | |
| | CTCCTTTCGCTTTCTTCCCTTCCTTTCTCGCCACGTTCGC | |
| | CGGCTTTCCCCGTCAAGCTCTAAATCGGGGGCTCCCTTTA | |
| | GGGTTCCGATTTAGTGCTTTACGGCACCTCGACCCCAAAA | |
| | AACTTGATTAGGGTGATGGTTCACGTAGTGGGCCATCGCC | |
| | CTGATAGACGGTTTTTCGCCCTTTGACGTTGGAGTCCACG | |
| | TTCTTTAATAGTGGACTCTTGTTCCAAACTGGAACAACAC | |
| | TCAACCCTATCTCGGTCTATTCTTTTGATTTATAAGGGAT | |
| | TTTGCCGATTTCGGCCTATTGGTTAAAAAATGAGCTGATT | |
| | TAACAAAAATTTAACGCGAATTTTAACAAAATTCAGGGCG | |
| | CAAGGGCTGCTAAAGGAAGCGGAACACGTAGAAAGCCAGT | |
| | CCGCAGAAACGGTGCTGACCCCGGATGAATGTCAGCTACT | |
| | GGGCTATCTGGACAAGGGAAAACGCAAGCGCAAAGAGAAA | |
| | GCAGGTAGCTTGCAGTGGGCTTACATGGCGATAGCTAGAC | |
| | TGGGCGGTTTTATGGACAGCAAGCGAACCGGAATTGCCAG | |
| | CTGGGGCGCCCTCTGGTAAGGTTGGGAAGCCCTGCAAAGT | |
| | AAACTGGATGGCTTTCTTGCCGCCAAGGATCTGATGGCGC | |
| | AGGGGATCAAGATCTGATCAAGAGACAGGATGAGGATCGT | |
| | TTCGCATGATTGAACAAGATGGATTGCACGCAGGTTCTCC | |
| | GGCCGCTTGGGTGGAGAGGCTATTCGGCTATGACTGGGCA | |
| | CAACAGACAATCGGCTGCTCTGATGCCGCCGTGTTCCGGC | |
| | TGTCAGCGCAGGGGCGCCCGGTTCTTTTTGTCAAGACCGA | |
| | CCTGTCCGGTGCCCTGAATGAACTGCAGGACGAGGCAGCG | |
| | CGGCTATCGTGGCTGGCCACGACGGGCGTTCCTTGCGCAG | |
| | CTGTGCTCGACGTTGTCACTGAAGCGGGAAGGGACTGGCT | |
| | GCTATTGGGCGAAGTGCCGGGGCAGGATCTCCTGTCATCC | |
| | CACCTTGCTCCTGCCGAGAAAGTATCCATCATGGCTGATG | |
| | CAATGCGGCGGCTGCATACGCTTGATCCGGCTACCTGCCC | |
| | ATTCGACCACCAAGCGAAACATCGCATCGAGCGAGCACGT | |
| | ACTCGGATGGAAGCCGGTCTTGTCGATCAGGATGATCTGG | |
| | ACGAAGAGCATCAGGGGCTCGCGCCAGCCGAACTGTTCGC | |
| | CAGGCTCAAGGCGCGCATGCCCGACGGCGAGGATCTCGTC | |
| | GTGACCCATGGCGATGCCTGCTTGCCGAATATCATGGTGG | |
| | AAAATGGCCGCTTTTCTGGATTCATCGACTGTGGCCGGCT | |
| | GGGTGTGGCGGACCGCTATCAGGACATAGCGTTGGCTACC | |
| | CGTGATATTGCTGAAGAGCTTGGCGGCGAATGGGCTGACC | |
| | GCTTCCTCGTGCTTTACGGTATCGCCGCTCCCGATTCGCA | |
| | GCGCATCGCCTTCTATCGCCTTCTTGACGAGTTCTTCTGA | |
| | ATTGAAAAAGGAAGAGTATGAGTATTCAACATTTCCGTGT | |
| | CGCCCTTATTCCCTTTTTTGCGGCATTTTGCCTTCCTGTT | |
| | TTTGCTCACCCAGAAACGCTGGTGAAAGTAAAAGATGCTG | |
| | AAGATCAGTTGGGTGCACGAGTGGGTTACATCGAACTGGA | |
| | TCTCAACAGCGGTAAGATCCTTGAGAGTTTTCGCCCCGAA | |
| | GAACGTTTTCCAATGATGAGCACTTTTAAAGTTCTGCTAT | |
| | GTGGCGCGGTATTATCCCGTATTGACGCCGGGCAAGAGCA | |
| | ACTCGGTCGCCGCATACACTATTCTCAGAATGACTTGGTT | |
| | GAGTACTCACCAGTCACAGAAAAGCATCTTACGGATGGCA | |
| | TGACAGTAAGAGAATTATGCAGTGCTGCCATAACCATGAG | |
| | TGATAACACTGCGGCCAACTTACTTCTGACAACGATCGGA | |
| | GGACCGAAGGAGCTAACCGCTTTTTTGCACAACATGGGGG | |
| | ATCATGTAACTCGCCTTGATCGTTGGGAACCGGAGCTGAA | |
| | TGAAGCCATACCAAACGACGAGCGTGACACCACGATGCCT | |
| | GTAGCAATGGCAACAACGTTGCGCAAACTATTAACTGGCG | |
| | AACTACTTACTCTAGCTTCCCGGCAACAATTAATAGACTG | |
| | GATGGAGGCGGATAAAGTTGCAGGACCACTTCTGCGCTCG | |
| | GCCCTTCCGGCTGGCTGGTTTATTGCTGATAAATCTGGAG | |
| | CCGGTGAGCGTGGGTCTCGCGGTATCATTGCAGCACTGGG | |
| | GCCAGATGGTAAGCCCTCCCGTATCGTAGTTATCTACACG | |
| | ACGGGGAGTCAGGCAACTATGGATGAACGAAATAGACAGA | |
| | TCGCTGAGATAGGTGCCTCACTGATTAAGCATTGGTAACT | |
| | GTCAGACCAAGTTTACTCATATATACTTTAGATTGATTTA | |
| | AAACTTCATTTTTAATTTAAAAGGATCTAGGTGAAGATCC | |
| | TTTTTGATAATCTCATGACCAAAATCCCTTAACGTGAGTT | |
| | TTCGTTCCACTGAGCGTCAGACCCCGTAGAAAAGATCAAA | |
| | GGATCTTCTTGAGATCCTTTTTTTCTGCGCGTAATCTGCT | |
| | GCTTGCAAACAAAAAAACCACCGCTACCAGCGGTGGTTTG | |
| | TTTGCCGGATCAAGAGCTACCAACTCTTTTTCCGAAGGTA | |
| | ACTGGCTTCAGCAGAGCGCAGATACCAAATACTGTTCTTC | |
| | TAGTGTAGCCGTAGTTAGGCCACCACTTCAAGAACTCTGT | |
| | AGCACCGCCTACATACCTCGCTCTGCTAATCCTGTTACCA | |
| | GTGGCTGCTGCCAGTGGCGATAAGTCGTGTCTTACCGGGT | |
| | TGGACTCAAGACGATAGTTACCGGATAAGGCGCAGCGGTC | |
| | GGGCTGAACGGGGGGTTCGTGCACACAGCCCAGCTTGGAG | |
| | CGAACGACCTACACCGAACTGAGATACCTACAGCGTGAGC | |
| | TATGAGAAAGCGCCACGCTTCCCGAAGGGAGAAAGGCGGA | |
| | CAGGTATCCGGTAAGCGGCAGGGTCGGAACAGGAGAGCGC | |
| | ACGAGGGAGCTTCCAGGGGGAAACGCCTGGTATCTTTATA | |
| | GTCCTGTCGGGTTTCGCCACCTCTGACTTGAGCGTCGATT | |
| | TTTGTGATGCTCGTCAGGGGGGCGGAGCCTATGGAAAAAC | |
| | GCCAGCAACGCGGCCTTTTTACGGTTCCTGGCCTTTTGCT | |
| | GGCCTTTTGCTCACATGTTCTTTCCTGCGTTATCCCCTGA | |
| | TTCTGTGGATAACCGTATTACCGCCTTTGAGTGAGCTGAT | |
| | ACCGCTCGCCGCAGCCGAACGACCGAGCGCAGCGAGTCAG | |
| | TGAGCGAGG | |
| MB9 | AAGCGGAAGAGCGCCCAATACGCAAACCGCCTCTCCCCGC | 2 |
| 894 | GCGTTGGCCGATTCATTAATGCAGCTGGCACGACAGGTTT | |
| | CCCGACTGGAAAGCGGGCAGTGAGCGCAACGCAATTAATG | |
| | TGAGTTAGCTCACTCATTAGGCACCCCAGGCTTTACACTT | |
| | TATGCTTCCGGCTCGTATGTTGTGTGGAATTGTGAGCGGA | |
| | TAACAATTTCACACAGGAAACAGCTATGACCATGATTACG | |
| | CCAAGCTTGGTACCAGAGACGGGTTGGCGGCGTATTTGTG | |
| | TCCCAAAAAACAGCCCCAATTGCCCCAATTGACCCCAAAT | |
| | TGACCCAGTAGCGGGCCCAACCCCGGCGAGAGCCCCCTTC | |
| | ACCCCACATATCAAACCTCCCCCGGTTCCCACACTTGCCG | |
| | TTAAGGGCGTAGGGTACTGCAGTCTGGAATCTACGCTTGT | |
| | TCAGACTTTGTACTAGTTTCTTTGTCTGGCCATCCGGGTA | |
| | ACCCATGCCGGACGCAAAATAGACTACTGAAAATTTTTTT | |
| | GCTTTGTGGTTGGGACTTTAGCCAAGGGTATAAAAGACCA | |
| | CCGTCCCCGAATTACCTTTCCTCTTCTTTTCTCTCTCTCC | |
| | TTGTCAACTCACACCCGAAATCGTTAAGCATTTCCTTCTG | |
| | AGTATAAGAATCATTCGCTAGCCCACAAAAATGACCCGAT | |
| | CCAAGTTCACCGAGTACAAGCAGGTTCTCCGAAACTCTTG | |
| | TGTTGGCAAGCCCCAGTACACTGAGAAGGATTACCCCGAT | |
| | CTCGACGGAAAGAACTTTCTCGTCACTGGTGCTACTGGTG | |
| | GAGTTGGTCTGGAGGCCACTAAGCTGCTCCTTGAGAAGAA | |
| | GTCTCACGTTATTATGGTTGGCCGATCTAAGACTAAGTCT | |
| | CAGTCTACCCTTGACGAGCTTCAGAAGACTTACTCTCACG | |
| | GTACTTTCGATTTCGTTGAGGCTGATCTCTCTGACCTTAC | |
| | CACTGTTGAGCGAGCCGGAGAGTACATTCGATCTAAGTAC | |
| | ACTACCCTTGACGGTGCCATTCTTAACGCTGGAGTCATGG | |
| | CTCCCCCCTACTCTCTCACTCCCCAGGGTCACGAGTCTCA | |
| | GTGGGGTATTAACGTTGTTGCTCACTTTCTCCTGTCTAAG | |
| | TACATCTCTCCTGCTCTGATTTCTGCTGCTCAGACTGCCC | |
| | CCAAGGACACCGTTCGACTTGTTTGGGTGTCTTCTTCTGT | |
| | CGTTGCCATGTCTCCTTACGAGGGAGGTATTAAGTTTGAC | |
| | GATATTAACCACTCTAAGGTCAAGAACCCTTCTCCTTGGA | |
| | CCCTTTACTCTCAGTCTAAGATTGGTGATGCTTACCTGGC | |
| | TTACCTTTGGTCTAAGCACCACCCCGATTCTGGAGTCCTT | |
| | TCTGTCTCTCTGGACCCCGGTAACCTGGCTTCTGATCTCT | |
| | CTCGACACACTTCTTGGCTTTCTTCTATTAAGAACTACGT | |
| | TCTTTACCCTCCCAAGTACGGTGCCTACACTGAGCTGTCT | |
| | GCTCTTCTCAACCCTTCCGTTAAGAACAACGAGCACCTTA | |
| | TTCCCTGGGGTATTGAGGGACACCTTCGACAGGATGTCGA | |
| | CGATGGTCGACGAGGAAAGGACGGAGAGGAGCTCTGGCAG | |
| | GGTCTTAACAAGGATGTCGAGGGTTTCTTTAAGGAGGAGT | |
| | AAACGCGTGCAATTAACAGATAGTTTGCCGGTGATAATTC | |
| | TCTTAACCTCCCACACTCCTTTGACATAACGATTTATGTA | |
| | ACGAAACTGAAATTTGACCAGATATTGTTGTAAATAGAAA | |
| | ATCTGGCTTGTAGGTGGCAAACTAGTGGTGTGTTCTGTGG | |
| | AGCATTCTCACTTTTGGTAAACGACATTGCTTCAAGTGCA | |
| | GCGGAATCAAAAAGTATAAAGTGGGCAGCGAGTATACCTG | |
| | TACAGACTGTAGGCGATAACTCAATCCAATTACCCCCCAC | |
| | AACATGACTGGCCAAACTGATCTCAAGACTTTATTGAAAT | |
| | CAGCAACACCGATTCTCAATGAAGGCACATACTTCTTCTG | |
| | CAACATTCACTTGACGCCTAAAGTTGGTGAGAAATGGACC | |
| | GACAAGACATATTCTGCTATCCACGGACTGTTGCCTGTGT | |
| | CGGTGGCTACAATACGTGAGTCAGAAGGGCTGACGGTGGT | |
| | GGTTCCCAAGGAAAAGGTCGACGAGTATCTGTCTGACTCG | |
| | TCATTGCCGCCTTTGGAGTACGACTCCAACTATGAGTGTG | |
| | CTTGGATCACTTTGACGATACATTCTTCGTTGGAGGCTGT | |
| | GGGTCTGACAGCTGCGTTTTCGGCGCGGTTGGCCGACAAC | |
| | AATATCAGCTGCAACGTCATTGCTGGCTTTCATCATGATC | |
| | ACATTTTTGTCGGCAAAGGCGACGCCCAGAGAGCCATTGA | |
| | CGTTCTTTCTAATTTGGACCGATAGCCGTATAGTCCAGTC | |
| | TATCTATAAGTTCAACTAACTCGTAACTATTACCATAACA | |
| | TATACTTCACTGCCCCAGATAAGGTTCCGATAAAAAGTTC | |
| | TGCAGACTAAATTTATTTCAGTCTCCTCTTCACCACCAAA | |
| | ATGCCCTCCTACGAAGCTCGAGCTAACGTCCACAAGTCCG | |
| | CCTTTGCCGCTCGAGTGCTCAAGCTCGTGGCAGCCAAGAA | |
| | AACCAACCTGTGTGCTTCTCTGGATGTTACCACCACCAAG | |
| | GAGCTCATTGAGCTTGCCGATAAGGTCGGACCTTATGTGT | |
| | GCATGATCAAGACCCATATCGACATCATTGACGACTTCAC | |
| | CTACGCCGGCACTGTGCTCCCCCTCAAGGAACTTGCTCTT | |
| | AAGCACGGTTTCTTCCTGTTCGAGGACAGAAAGTTCGCAG | |
| | ATATTGGCAACACTGTCAAGCACCAGTACAAGAACGGTGT | |
| | CTACCGAATCGCCGAGTGGTCCGATATCACCAACGCCCAC | |
| | GGTGTACCCGGAACCGGAATCATTGCTGGCCTGCGAGCTG | |
| | GTGCCGAGGAAACTGTCTCTGAACAGAAGAAGGAGGACGT | |
| | CTCTGACTACGAGAACTCCCAGTACAAGGAGTTCCTGGTC | |
| | CCCTCTCCCAACGAGAAGCTGGCCAGAGGTCTGCTCATGC | |
| | TGGCCGAGCTGTCTTGCAAGGGCTCTCTGGCCACTGGCGA | |
| | GTACTCCAAGCAGACCATTGAGCTTGCCCGATCCGACCCC | |
| | GAGTTTGTGGTTGGCTTCATTGCCCAGAACCGACCTAAGG | |
| | GCGACTCTGAGGACTGGCTTATTCTGACCCCCGGGGTGGG | |
| | TCTTGACGACAAGGGAGACGCTCTCGGACAGCAGTACCGA | |
| | ACTGTTGAGGATGTCATGTCTACCGGAACGGATATCATAA | |
| | TTGTCGGCCGAGGTCTGTACGGCCAGAACCGAGATCCTAT | |
| | TGAGGAGGCCAAGCGATACCAGAAGGCTGGCTGGGAGGCT | |
| | TACCAGAAGATTAACTGTTAGAGGTTAGACTATGGATATG | |
| | TAATTTAACTGTGTATATAGAGAGCGTGCAAGTATGGAGC | |
| | GCTTGTTCAGCTTGTATGATGGTCAGACGACCTGTCTGAT | |
| | CGAGTATGTATGATACTGCACAACCTGTGTATCCGCATGA | |
| | TCTGTCCAATGGGGCATGTTGTTGTGTTTCTCGATACGGA | |
| | GATGCTGGGTACAAGTAGCTAATACGATTGAACTACTTAT | |
| | ACTTATATGAGGCTTGAAGAAAGCTGACTTGTGTATGACT | |
| | TATTCTCAACTACATCCCCAGTCACAATACCACCACTGCA | |
| | CTACCACTACACCTCGAGCATGCATCTAGTGGTGTGTTCT | |
| | GTGGAGCATTCTCACTTTTGGTAAACGACATTGCTTCAAG | |
| | TGCAGCGGAATCAAAAAGTATAAAGTGGGCAGCGAGTATA | |
| | CCTGTACAGACTGTAGGCGATAACTCAATCCAATTACCCC | |
| | CCACAACATGACTGGCCAAACTGATCTCAAGACTTTATTG | |
| | AAATCAGCAACACCGATTCTCAATGAAGGCACATACTTCT | |
| | TCTGCAACATTCACTTGACGCCTAAAGTTGGTGAGAAATG | |
| | GACCGACAAGACATATTCTGCTATCCACGGACTGTTGCCT | |
| | GTGTCGGTGGCTACAATACGTGAGTCAGAAGGGCTGACGG | |
| | TGGTGGTTCCCAAGGAAAAGGTCGACGAGTATCTGTCTGA | |
| | CTCGTCATTGCCGCCTTTGGAGTACGACTCCAACTATGAG | |
| | TGTGCTTGGATCACTTTGACGATACATTCTTCGTTGGAGG | |
| | CTGTGGGTCTGACAGCTGCGTTTTCGGCGCGGTTGGCCGA | |
| | CAACAATATCAGCTGCAACGTCATTGCTGGCTTTCATCAT | |
| | GATCACATTTTTGTCGGCAAAGGCGACGCCCAGAGAGCCA | |
| | TTGACGTTCTTTCTAATTTGGACCGATAGCCGTATAGTCC | |
| | AGTCTATCTATAAGTTCAACTAACTCGTAACTATTACCAT | |
| | AACATATACTTCACTGCCCCAGATAAGGTTCCGATAAAAA | |
| | GTTCTGCAGACTAAATTTATTTCAGTCTCCTCTTCACCAC | |
| | CAAAATGCCCTCCTACGAAGCTCGAGCTAACGTCCACAAG | |
| | TCCGCCTTTGCCGCTCGAGTGCTCAAGCTCGTGGCAGCCA | |
| | AGAAAACCAACCTGTGTGCTTCTCTGGATGTTACCACCAC | |
| | CAAGGGGCCGCTCGAGCATGCATCTAGAGGGCCCAATTCG | |
| | CCCTATAGTGAGTCGTATTACAATTCACTGGCCGTCGTTT | |
| | TACAACGTCGTGACTGGGAAAACCCTGGCGTTACCCAACT | |
| | TAATCGCCTTGCAGCACATCCCCCTTTCGCCAGCTGGCGT | |
| | AATAGCGAAGAGGCCCGCACCGATCGCCCTTCCCAACAGT | |
| | TGCGCAGCCTGAATGGCGAATGGACGCGCCCTGTAGCGGC | |
| | GCATTAAGCGCGGCGGGTGTGGTGGTTACGCGCAGCGTGA | |
| | CCGCTACACTTGCCAGCGCCCTAGCGCCCGCTCCTTTCGC | |
| | TTTCTTCCCTTCCTTTCTCGCCACGTTCGCCGGCTTTCCC | |
| | CGTCAAGCTCTAAATCGGGGGCTCCCTTTAGGGTTCCGAT | |
| | TTAGTGCTTTACGGCACCTCGACCCCAAAAAACTTGATTA | |
| | GGGTGATGGTTCACGTAGTGGGCCATCGCCCTGATAGACG | |
| | GTTTTTCGCCCTTTGACGTTGGAGTCCACGTTCTTTAATA | |
| | GTGGACTCTTGTTCCAAACTGGAACAACACTCAACCCTAT | |
| | CTCGGTCTATTCTTTTGATTTATAAGGGATTTTGCCGATT | |
| | TCGGCCTATTGGTTAAAAAATGAGCTGATTTAACAAAAAT | |
| | TTAACGCGAATTTTAACAAAATTCAGGGCGCAAGGGCTGC | |
| | TAAAGGAAGCGGAACACGTAGAAAGCCAGTCCGCAGAAAC | |
| | GGTGCTGACCCCGGATGAATGTCAGCTACTGGGCTATCTG | |
| | GACAAGGGAAAACGCAAGCGCAAAGAGAAAGCAGGTAGCT | |
| | TGCAGTGGGCTTACATGGCGATAGCTAGACTGGGCGGTTT | |
| | TATGGACAGCAAGCGAACCGGAATTGCCAGCTGGGGCGCC | |
| | CTCTGGTAAGGTTGGGAAGCCCTGCAAAGTAAACTGGATG | |
| | GCTTTCTTGCCGCCAAGGATCTGATGGCGCAGGGGATCAA | |
| | GATCTGATCAAGAGACAGGATGAGGATCGTTTCGCATGAT | |
| | TGAACAAGATGGATTGCACGCAGGTTCTCCGGCCGCTTGG | |
| | GTGGAGAGGCTATTCGGCTATGACTGGGCACAACAGACAA | |
| | TCGGCTGCTCTGATGCCGCCGTGTTCCGGCTGTCAGCGCA | |
| | GGGGCGCCCGGTTCTTTTTGTCAAGACCGACCTGTCCGGT | |
| | GCCCTGAATGAACTGCAGGACGAGGCAGCGCGGCTATCGT | |
| | GGCTGGCCACGACGGGCGTTCCTTGCGCAGCTGTGCTCGA | |
| | CGTTGTCACTGAAGCGGGAAGGGACTGGCTGCTATTGGGC | |
| | GAAGTGCCGGGGCAGGATCTCCTGTCATCCCACCTTGCTC | |
| | CTGCCGAGAAAGTATCCATCATGGCTGATGCAATGCGGCG | |
| | GCTGCATACGCTTGATCCGGCTACCTGCCCATTCGACCAC | |
| | CAAGCGAAACATCGCATCGAGCGAGCACGTACTCGGATGG | |
| | AAGCCGGTCTTGTCGATCAGGATGATCTGGACGAAGAGCA | |
| | TCAGGGGCTCGCGCCAGCCGAACTGTTCGCCAGGCTCAAG | |
| | GCGCGCATGCCCGACGGCGAGGATCTCGTCGTGACCCATG | |
| | GCGATGCCTGCTTGCCGAATATCATGGTGGAAAATGGCCG | |
| | CTTTTCTGGATTCATCGACTGTGGCCGGCTGGGTGTGGCG | |
| | GACCGCTATCAGGACATAGCGTTGGCTACCCGTGATATTG | |
| | CTGAAGAGCTTGGCGGCGAATGGGCTGACCGCTTCCTCGT | |
| | GCTTTACGGTATCGCCGCTCCCGATTCGCAGCGCATCGCC | |
| | TTCTATCGCCTTCTTGACGAGTTCTTCTGAATTGAAAAAG | |
| | GAAGAGTATGAGTATTCAACATTTCCGTGTCGCCCTTATT | |
| | CCCTTTTTTGCGGCATTTTGCCTTCCTGTTTTTGCTCACC | |
| | CAGAAACGCTGGTGAAAGTAAAAGATGCTGAAGATCAGTT | |
| | GGGTGCACGAGTGGGTTACATCGAACTGGATCTCAACAGC | |
| | GGTAAGATCCTTGAGAGTTTTCGCCCCGAAGAACGTTTTC | |
| | CAATGATGAGCACTTTTAAAGTTCTGCTATGTGGCGCGGT | |
| | ATTATCCCGTATTGACGCCGGGCAAGAGCAACTCGGTCGC | |
| | CGCATACACTATTCTCAGAATGACTTGGTTGAGTACTCAC | |
| | CAGTCACAGAAAAGCATCTTACGGATGGCATGACAGTAAG | |
| | AGAATTATGCAGTGCTGCCATAACCATGAGTGATAACACT | |
| | GCGGCCAACTTACTTCTGACAACGATCGGAGGACCGAAGG | |
| | AGCTAACCGCTTTTTTGCACAACATGGGGGATCATGTAAC | |
| | TCGCCTTGATCGTTGGGAACCGGAGCTGAATGAAGCCATA | |
| | CCAAACGACGAGCGTGACACCACGATGCCTGTAGCAATGG | |
| | CAACAACGTTGCGCAAACTATTAACTGGCGAACTACTTAC | |
| | TCTAGCTTCCCGGCAACAATTAATAGACTGGATGGAGGCG | |
| | GATAAAGTTGCAGGACCACTTCTGCGCTCGGCCCTTCCGG | |
| | CTGGCTGGTTTATTGCTGATAAATCTGGAGCCGGTGAGCG | |
| | TGGGTCTCGCGGTATCATTGCAGCACTGGGGCCAGATGGT | |
| | AAGCCCTCCCGTATCGTAGTTATCTACACGACGGGGAGTC | |
| | AGGCAACTATGGATGAACGAAATAGACAGATCGCTGAGAT | |
| | AGGTGCCTCACTGATTAAGCATTGGTAACTGTCAGACCAA | |
| | GTTTACTCATATATACTTTAGATTGATTTAAAACTTCATT | |
| | TTTAATTTAAAAGGATCTAGGTGAAGATCCTTTTTGATAA | |
| | TCTCATGACCAAAATCCCTTAACGTGAGTTTTCGTTCCAC | |
| | TGAGCGTCAGACCCCGTAGAAAAGATCAAAGGATCTTCTT | |
| | GAGATCCTTTTTTTCTGCGCGTAATCTGCTGCTTGCAAAC | |
| | AAAAAAACCACCGCTACCAGCGGTGGTTTGTTTGCCGGAT | |
| | CAAGAGCTACCAACTCTTTTTCCGAAGGTAACTGGCTTCA | |
| | GCAGAGCGCAGATACCAAATACTGTTCTTCTAGTGTAGCC | |
| | GTAGTTAGGCCACCACTTCAAGAACTCTGTAGCACCGCCT | |
| | ACATACCTCGCTCTGCTAATCCTGTTACCAGTGGCTGCTG | |
| | CCAGTGGCGATAAGTCGTGTCTTACCGGGTTGGACTCAAG | |
| | ACGATAGTTACCGGATAAGGCGCAGCGGTCGGGCTGAACG | |
| | GGGGGTTCGTGCACACAGCCCAGCTTGGAGCGAACGACCT | |
| | ACACCGAACTGAGATACCTACAGCGTGAGCTATGAGAAAG | |
| | CGCCACGCTTCCCGAAGGGAGAAAGGCGGACAGGTATCCG | |
| | GTAAGCGGCAGGGTCGGAACAGGAGAGCGCACGAGGGAGC | |
| | TTCCAGGGGGAAACGCCTGGTATCTTTATAGTCCTGTCGG | |
| | GTTTCGCCACCTCTGACTTGAGCGTCGATTTTTGTGATGC | |
| | TCGTCAGGGGGGCGGAGCCTATGGAAAAACGCCAGCAACG | |
| | CGGCCTTTTTACGGTTCCTGGCCTTTTGCTGGCCTTTTGC | |
| | TCACATGTTCTTTCCTGCGTTATCCCCTGATTCTGTGGAT | |
| | AACCGTATTACCGCCTTTGAGTGAGCTGATACCGCTCGCC | |
| | GCAGCCGAACGACCGAGCGCAGCGAGTCAGTGAGCGAGG | |
| MB9 | | 3 |
| 523 | | |
| MB7 | TCTGGTCCCGCCGCATCCATACCGCCAGTTGTTTACCCTC | 4 |
| 270 | ACAACGTTCCAGTAACCGGGCATGTTCATCATCAGTAACC | |
| | CGTATCGTGAGCATCCTCTCTCGTTTCATCGGTATCATTA | |
| | CCCCCATGAACAGAAATTCCCCCTTACACGGAGGCATCAA | |
| | GTGACCAAACAGGAAAAAACCGCCCTTAACATGGCCCGCT | |
| | TTATCAGAAGCCAGACATTAACGCTTCTGGAGAAACTCAA | |
| | CGAGCTGGACGCGGATGAACAGGCAGACATCTGTGAATCG | |
| | CTTCACGACCACGCTGATGAGCTTTACCGCAGGTGGGCCA | |
| | TTCTCATGAAGAATATCTTGAATTTATTGTCATATTACTA | |
| | GTTGGTGTGGAAGTCCATATATCGGTGATCAATATAGTGG | |
| | TTGACATGCTGGCTAGTCAACATTGAGCCTTTTGATCATG | |
| | CAAATATATTACGGTATTTTACAATCAAATATCAAACTTA | |
| | ACTATTGACTTTATAACTTATTTAGGTGGTAACATTCTTA | |
| | TAAAAAAGAAAAAAATTACTGCAAAACAGTACTAGCTTTT | |
| | AACTTGTATCCTAGGTTATCTATGCTGTCTCACCATAGAG | |
| | AATATTACCTATTTCAGAATGTATGTCCATGATTCGCCGG | |
| | GTAAATACATATAATACACAAATCTGGCTTAATAAAGTCT | |
| | ATAATATATCTCATAAAGAAGTGCTAAATTGGCTAGTGCT | |
| | ATATATTTTTAAGAAAATTTCTTTTGACTAAGTCCATATC | |
| | GACTTTGTAAAAGTTCACTTTAGCATACATATATTACACG | |
| | AGCCAGAAATTGTAACTTTTGCCTAAAATCACAAATTGCA | |
| | AAATTTAATTGCTTGCAAAAGGTCACATGCTTATAATCAA | |
| | CTTTTTTAAAAATTTAAAATACTTTTTTATTTTTTATTTT | |
| | TAAACATAAATGAAATAATTTATTTATTGTTTATGATTAC | |
| | CGAAACATAAAACCTGCTCAAGAAAAAGAAACTGTTTTGT | |
| | CCTTGGAAAAAAAGCACTACCTAGGAGCGGCCAAAATGCC | |
| | GAGGCTTTCATAGCTTAAACTCTTTACAGAAAATAGGCAT | |
| | TATAGATCAGTTCGAGTTTTCTTATTCTTCCTTCCGGTTT | |
| | TATCGTCACAGTTTTACAGTAAATAAGTATCACCTCTTAG | |
| | AGTTAACTATGAGATAAGCAAGTATCATCTCATTTCATTT | |
| | ACCTGAAGTCGAGTAAACAGAAAATCCAATTGTTGATGAA | |
| | CCTCAATGACTTAGAACTATCTATCGGCAGATCATATAAA | |
| | GAGGATTTAGGTACCTAGAGGACTGTACCTGGAGTATATA | |
| | TATATATATATATATATTATCTCAACTATAGTCCATAGAG | |
| | GTTTCTTTCTTGAGGCCTTAAACTGCTAAAGAATGATATT | |
| | GGTGGAATGCAAGCACCAATCTCTCTTCTTTCGTAACTGT | |
| | TCATATACTTCAAACCAAGAATGTAACGGGCATTGACCCA | |
| | TCCAAAACCTTCAGTAGCTGCCCCTTTAAAGTCAGCACCT | |
| | TGATTACCGTATTCTGCTTCAACACGATGAGGATCTGTTC | |
| | CTCTTGTGACATCATATTTTTCAACCACAATACCATTATA | |
| | ATCGACAAAAGCCTTTGTCATCATGAAAAGCCATCTATAA | |
| | GCTAGCCTATTCGTTACAGTTAAATAACCATAAGAACGGA | |
| | GGCCTTCCCAAGCAAGAATTTGATGGGGTGCCCAACCAAA | |
| | TGGATAGTCCCATTGTCTAATTGGTCTCGAAATAGAAATT | |
| | GGGCCTCGAGAACGCTCCGTACATGCAGCTAAACCTCCAA | |
| | GCATCTCTAACTTGGGTAGTGCTTTCTCCACCATTTTCTG | |
| | TGCTTGCTCCTTCGTGGCAAGTCCAGCCCATAATGCCCAG | |
| | AATGTAGTTGCGGATTCGTATGACGTTCTGTGCTTGATTT | |
| | TTGTGTTGTAGTCAAAGAAAAACCCCGACTCGTCATCCCA | |
| | CATATATTTGGTAATTGATGAGGCAACGCTAATTATCAAC | |
| | ATATAGATTGTTATCTATCTGCATGAACACGAAATCTTTA | |
| | CTTGACGACTTGAGGCTGATGGTGTTTATGCAAAGAAACC | |
| | ACTGTGTTTAATATGTGTCACTGTTTGATATTACTGTCAG | |
| | CGTAGAAGATAATAGTAAAAGCGGTTAATAAGTGTATTTG | |
| | AGATAAGTGTGATAAAGTTTTTACAGCGAAAAGACGATAA | |
| | ATACAAGAAAATGATTACGAGGATACGGAGAGAGGTATGT | |
| | ACATGTGTATTTATATACTAAGCTGCCGGCGGTTGTTTGC | |
| | AAGACCGAGAAAAGGCTAGCAAGAATCGGGTCATTGTAGC | |
| | GTATGCGCCTGTGAACATTCTCTTCAACAAGTTTGATTCC | |
| | ATTGCGGTGAAATGGTAAAAGTCAACCCCCTGCGATGTAT | |
| | ATTTTCCTGTACAATCAATCAAAAAGCCAAATGATTTAGC | |
| | ATTATCTTTACATCTTGTTATTTTACAGATTTTATGTTTA | |
| | GATCTTTTATGCTTGCTTTTCAAAAGGCCTGCAGGCAAGT | |
| | GCACAAACAATACTTAAATAAATACTACTCAGTAATAACC | |
| | TATTTCTTAGCATTTTTGACGAAATTTGCTATTTTGTTAG | |
| | AGTCTTTTACACCATTTGTCTCCACACCTCCGCTTACATC | |
| | AACACCAATAACGCCATTTAATCTAAGCGCATCACCAACA | |
| | TTTTCTGGCGTCAGTCCACCAGCTAACATAAAATGTAAGC | |
| | TCTGCCTCGCGCGTTTCGGTGATGACGGTGAAAACCTCTG | |
| | ACACATGCAGCTCCCGGAGACGGTCACAGCTTGTCTGTAA | |
| | GCGGATGCCGGGAGCAGACAAGCCCGTCAGGGCGCGTCAG | |
| | CGGGTGTTGGCGGGTGTCGGGGCGCAGCCATGACCCAGTC | |
| | ACGTAGCGATAGCGGAGTGTATACTGGCTTAACTATGCGG | |
| | CATCAGAGCAGATTGTACTGAGAGTGCACCATATGCGGTG | |
| | TGAAATACCGCACAGATGCGTAAGGAGAAAATACCGCATC | |
| | AGGCGCTCTTCCGCTTCCTCGCTCACTGACTCGCTGCGCT | |
| | CGGTCGTTCGGCTGCGGCGAGCGGTATCAGCTCACTCAAA | |
| | GGCGGTAATACGGTTATCCACAGAATCAGGGGATAACGCA | |
| | GGAAAGAACATGTGAGCAAAAGGCCAGCAAAAGGCCAGGA | |
| | ACCGTAAAAAGGCCGCGTTGCTGGCGTTTTTCCATAGGCT | |
| | CCGCCCCCCTGACGAGCATCACAAAAATCGACGCTCAAGT | |
| | CAGAGGTGGCGAAACCCGACAGGACTATAAAGATACCAGG | |
| | CGTTTCCCCCTGGAAGCTCCCTCGTGCGCTCTCCTGTTCC | |
| | GACCCTGCCGCTTACCGGATACCTGTCCGCCTTTCTCCCT | |
| | TCGGGAAGCGTGGCGCTTTCTCATAGCTCACGCTGTAGGT | |
| | ATCTCAGTTCGGTGTAGGTCGTTCGCTCCAAGCTGGGCTG | |
| | TGTGCACGAACCCCCCGTTCAGCCCGACCGCTGCGCCTTA | |
| | TCCGGTAACTATCGTCTTGAGTCCAACCCGGTAAGACACG | |
| | ACTTATCGCCACTGGCAGCAGCCACTGGTAACAGGATTAG | |
| | CAGAGCGAGGTATGTAGGCGGTGCTACAGAGTTCTTGAAG | |
| | TGGTGGCCTAACTACGGCTACACTAGAAGGACAGTATTTG | |
| | GTATCTGCGCTCTGCTGAAGCCAGTTACCTTCGGAAAAAG | |
| | AGTTGGTAGCTCTTGATCCGGCAAACAAACCACCGCTGGT | |
| | AGCGGTGGTTTTTTTGTTTGCAAGCAGCAGATTACGCGCA | |
| | GAAAAAAAGGATCTCAAGAAGATCCTTTGATCTTTTCTAC | |
| | GGGGTCTGACGCTCAGTGGAACGAAAACTCACGTTAAGGG | |
| | ATTTTGGTCATGAGATTATCAAAAAGGATCTTCACCTAGA | |
| | TCCTTTTAAATTAAAAATGAAGTTTTAAATCAATCTAAAG | |
| | TATATATGAGTAAACTTGGTCTGACAGTTACCAATGCTTA | |
| | ATCAGTGAGGCACCTATCTCAGCGATCTGTCTATTTCGTT | |
| | CATCCATAGTTGCCTGACTCCCCGTCGTGTAGATAACTAC | |
| | GATACGGGAGGGCTTACCATCTGGCCCCAGTGCTGCAATG | |
| | ATACCGCGAGACCCACGCTCACCGGCTCCAGATTTATCAG | |
| | CAATAAACCAGCCAGCCGGAAGGGCCGAGCGCAGAAGTGG | |
| | TCCTGCAACTTTATCCGCCTCCATCCAGTCTATTAATTGT | |
| | TGCCGGGAAGCTAGAGTAAGTAGTTCGCCAGTTAATAGTT | |
| | TGCGCAACGTTGTTGCCATTGCTGCAGGCATCGTGGTGTC | |
| | ACGCTCGTCGTTTGGTATGGCTTCATTCAGCTCCGGTTCC | |
| | CAACGATCAAGGCGAGTTACATGATCCCCCATGTTGTGCA | |
| | AAAAAGCGGTTAGCTCCTTCGGTCCTCCGATCGTTGTCAG | |
| | AAGTAAGTTGGCCGCAGTGTTATCACTCATGGTTATGGCA | |
| | GCACTGCATAATTCTCTTACTGTCATGCCATCCGTAAGAT | |
| | GCTTTTCTGTGACTGGTGAGTACTCAACCAAGTCATTCTG | |
| | AGAATAGTGTATGCGGCGACCGAGTTGCTCTTGCCCGGCG | |
| | TCAACACGGGATAATACCGCGCCACATAGCAGAACTTTAA | |
| | AAGTGCTCATCATTGGAAAACGTTCTTCGGGGCGAAAACT | |
| | CTCAAGGATCTTACCGCTGTTGAGATCCAGTTCGATGTAA | |
| | CCCACTCGTGCACCCAACTGATCTTCAGCATCTTTTACTT | |
| | TCACCAGCGTTTCTGGGTGAGCAAAAACAGGAAGGCAAAA | |
| | TGCCGCAAAAAAGGGAATAAGGGCGACACGGAAATGTTGA | |
| | ATACTCATACTCTTCCTTTTTCAATATTATTGAAGCATTT | |
| | ATCAGGGTTATTGTCTCATGAGCGGATACATATTTGAATG | |
| | TATTTAGAAAAATAAACAAATAGGGGTTCCGCGCACATTT | |
| | CCCCGAAAAGTGCCACCTGACGTCTAAGAAACCATTATTA | |
| | TCATGACATTAACCTATAAAAATAGGCGTATCACGAGGCC | |
| | CTTTCGTCTGGCCACCTAGGCCGGCCTCGCGGTCAGAAGG | |
| | GGCAGCTCTAAACGAAGAACTGCGGTCAGGTGACACAACT | |
| | TTTTCCATCTCAGGGTGTGTCGCGTGTGCTTCATCCAAAC | |
| | TTTAGTTGGGGTTCGGGTTCGCGCGAGATGATCACGTGCC | |
| | CTGATTTGGTGTCGTCCCCCGTCGCGCTGCGCACGTGATT | |
| | TATTTATTTCCGGTGGCTGCTGTCTACGCGGGGCCTTCTC | |
| | TGCCCTTCTGTTTCAACCTTCGGGCGGTTCTCGTAACCAG | |
| | CAGTAGCAATCCATTTCGAAACTCAAAGAGCTAAAAACGT | |
| | TAAACCTCAGCAGTCGCTCGACGAATGGGCTGCGGTTGGG | |
| | AAGCCCACGAGGCCTATAGCCAGAGCCTCGAGTTGACAGG | |
| | AGCCCAGACGCCTTTTCCAACGGCAACTTTTATATAAAAT | |
| | GGCAATGTATTCATGCAATTGCGGCCGTGTCAGGTTGGAG | |
| | ACACTGGACCACACTCTCCATTGCTTCCTGAGGAGATGGA | |
| | TCATTGCTAGTGCATCTACGCGCAGCAATCCCGCAAGCTC | |
| | GACAACCGTAGATGGGCTTTGGTGGGCCAATCAATTACGC | |
| | AACCCGCACGTTAAATTGTATGAGGAAGGAAGGCCACGGT | |
| | ACAAAGTGGGTGGTCTTCACCCAGTGGTTGTTGGTGGCGT | |
| | CATGCAGACCATGCATTGGGGATAGCACAGGGTTGGGGTG | |
| | TCTTGTGGACTCAATGGGTGAAAGGAGATGGAAAAGGGCG | |
| | GTGAAAAGTGGTAGAATCGAAATCCCTGACGTCAATTTAT | |
| | AAAGTAAAATGCGTTTCTGCCATTTTGCTCCCCTCCTTCT | |
| | TTCGCAATCGCCTCCCCAAAAGTTGTCGTGGCAGTACACA | |
| | TGCTTGCATACAATGAAGCTAATCCGGCTTGCTCAGTAGT | |
| | TGCTATATCCAGGCATGGTGTGAAACCCCTCAAAGTATAT | |
| | ATAGGAGCGGTGAGCCCCAGTCTGGGGTCTTTTCTCTCCA | |
| | TCTCAAAACTACTTTCTCACATGCTAGCCACAAAAATGGA | |
| | TTATAACAGCGCGGATTTCAAGGAGATCTGGGGCAAGGCC | |
| | GCCGACACCGCGCTGCTGGGACCGTACAACTACCTCGCCA | |
| | ACAACCGGGGCCACAACATCAGAGAACACTTGATCGCAGC | |
| | GTTCGGAGCGGTTATCAAGGTGGACAAGAGCGATCTCGAA | |
| | ACCATTTCGCACATCACCAAGATTTTGCATAACTCGTCGC | |
| | TGCTTGTTGATGACGTGGAAGACAACTCGATGCTCCGACG | |
| | AGGCCTGCCGGCAGCCCATTGTCTGTTTGGAGTCCCCCAA | |
| | ACCATCAACTCCGCCAACTACATGTACTTTGTGGCTCTGC | |
| | AGGAGGTGCTCAAGCTCAAGTCTTATGATGCCGTCTCCAT | |
| | TTTCACCGAGGAAATGATCAACTTGCATAGAGGTCAGGGT | |
| | ATGGATCTCTACTGGAGAGAAACACTCACTTGCCCCTCGG | |
| | AAGACGAGTATCTGGAGATGGTGGTGCACAAGACCGGAGG | |
| | ACTGTTTCGGCTGGCTCTGAGACTTATGCTGTCGGTGGCA | |
| | TCGAAACAGGAGGACCATGAAAAGATCAACTTTGATCTCA | |
| | CACACCTTACCGACACACTGGGAGTCATTTACCAGATTCT | |
| | GGATGATTACCTCAACCTGCAGTCCACGGAATTGACCGAG | |
| | AACAAGGGATTCTGCGAAGATATCAGCGAAGGAAAGTTTT | |
| | CGTTTCCGCTGATTCACAGCATCCGGACCAACCCGGATAA | |
| | CCACGAGATTCTCAACATTCTCAAACAGCGAACAAGCGAC | |
| | GCTTCACTCAAAAAGTACGCCGTGGACTACATGAGAACAG | |
| | AAACCAAGAGTTTCGACTACTGCCTCAAGAGAATCCAGGC | |
| | CATGTCACTCAAGGCAAGTTCGTACATTGATGATCTCGCA | |
| | GCAGCCGGCCACGATGTCTCCAAGTTGCGAGCCATTTTGC | |
| | ATTATTTTGTGTCCACCTCTGACTGTGAGGAGAGAAAGTA | |
| | CTTTGAGGATGCGCAGTGAACGCGTCTATCCGAAGATCAA | |
| | GAGCGAAGCAAGTIGTAAGTCCAGGACATGTTTCCCGCCC | |
| | ACGCGAGTGATTTATAACACCTCTCTTTTTTGACACCCGC | |
| | TCGCCTTGAAATTCATGTCACATAAATTATAGTCAACGAC | |
| | GTTTGAATAACTTGTCTTGTAGTTCGATGATGATCATATG | |
| | ATTACATTAATAGTAATTACTGTATTAGTGCAATCACATG | |
| | TTGCTACTGTACCTGCTGTGGACCACGCACGGCGGAACGT | |
| | ACCGTACAAATATTTTCTTGCTCACATGACTCTCTCTCGG | |
| | CCGCGCACGCCGGTGGCAAATTGCTCTTGCATTGGCTCTG | |
| | TCTCTAGACGTCCAAACCGTCCAAAGTGGCAGGGTGACGT | |
| | GATGCGACGCACGAAGGAGATGGCCCGGTGGCGAGGAACC | |
| | GGACACGGCGAGCCGGCGGGAAAAAAGGCGGAAAACGAAA | |
| | AGCGAAGGGCACAATCTGACGGTGCGGCTGCCACCAACCC | |
| | AAGGAGGCTATTTTGGGTCGCTTTCCATTTCACATTCGCC | |
| | CTCAATGGCCACTTTGCGGTGGTGAACATGGTTTCTGAAA | |
| | CAACCCCCCAGAATTAGAGTATATTGATGTGTTTAAGATT | |
| | GGGTTGCTATTTGGCCATTGTGGGGGAGGGTAGCGACGTG | |
| | GAGGACATTCCAGGGCGAATTGAGCCTAGAAAGTGGTACC | |
| | ATTCCAACCGTCTCAGTCGTCCGAATTGATCGCTATAACT | |
| | ATCACCTCTCTCACATGTCTACTTCCCCAACCAACATCCC | |
| | CAACCTCCCCCACACTAAAGTTCACGCCAATAATGTAGGC | |
| | ACTCTTTCTGGGTGTGGGACAGCAGAGCAATACGGAGGGG | |
| | AGATTACACAACGAGCCACAATTGGGGAGATGGTAGCCAT | |
| | CTCACTCGACCCGTCGACTTTTGGCAACGCTCAATTACCC | |
| | ACCAAATTTGGGCTGGAGTTGAGGGGACCGTGTTCCAGCG | |
| | CTGTAGGACCAGCAACACACACGGTATCAACAGCAACCAA | |
| | CGCCCCCGCTAATGCACCCAGTACTGCGCAGGTGTGGGCC | |
| | AGGTGCGTTCCAGATGCGAGTTGGCGAACCCTAAGCCGAC | |
| | AGTGTACTTTTTGGGACGGGCAGTAGCAATCGTGGGCGGA | |
| | GACCCCGGTGTATATAAAGGGGTGGAGAGGACGGATTATT | |
| | AGCACCAACACACACACTTATACTACATGCTAGCCACAAA | |
| | AATGTCCAAGAAACACATTGTCATTATCGGTGCTGGCGTG | |
| | GGTGGCACGGCTACAGCTGCTCGTTTGGCCCGCGAAGGCT | |
| | TCAAGGTCACTGTGGTGGAGAAAAACGACTTTGGTGGCGG | |
| | CCGCTGCTCCTTGATCCATCACCAGGGCCATCGCTTTGAT | |
| | CAGGGCCCGTCGCTCTACCTGATGCCCAAGTACTTTGAGG | |
| | ACGCCTTTGCCGATCTGGACGAGCGCATTCAAGACCACCT | |
| | GGAGCTGCTGCGATGCGACAACAACTACAAGGTGCACTTT | |
| | GACGACGGTGAGTCGATCCAGCTGTCGTCTGACTTGACAC | |
| | GCATGAAGGCTGAATTGGACCGCGTGGAGGGCCCCCTTGG | |
| | TTTTGGCCGATTCCTGGATTTCATGAAAGAGACACACATC | |
| | CACTACGAAAGCGGCACCCTGATTGCGCTCAAGAAGAATT | |
| | TCGAATCCATCTGGGACCTGATTCGCATCAAGTACGCTCC | |
| | AGAGATCTTTCGCTTGCACCTGTTTGGCAAGATCTACGAC | |
| | CGCGCTTCCAAGTACTTCAAGACCAAGAAGATGCGCATGG | |
| | CATTCACGTTTCAGACCATGTATATGGGCATGTCGCCCTA | |
| | CGATGCGCCTGCTGTCTACAGCCTGTTGCAGTACACCGAG | |
| | TTCGCTGAAGGCATCTGGTATCCCCGTGGCGGCTTCAACA | |
| | TGGTGGTTCAGAAGCTAGAGGCGATTGCAAAGCAAAAGTA | |
| | CGATGCCGAGTTTATCTACAATGCGCCTGTTGCCAAGATT | |
| | AACACCGATGATGCCACCAAACAAGTGACAGGTGTAACCT | |
| | TGGAAAATGGCCACATCATCGATGCCGATGCGGTTGTGTG | |
| | TAACGCAGATCTGGTCTATGCTTATCACAATCTGTTGCCT | |
| | CCCTGCCGATGGACGCAAAACACACTGGCTTCCAAGAAAT | |
| | TGACGTCTTCTTCCATTTCCTTCTACTGGTCCATGTCCAC | |
| | CAAGGTGCCTCAATTGGACGTGCACAACATCTTTTTGGCC | |
| | GAGGCTTATCAGGAGAGCTTTGACGAAATCTTCAAGGACT | |
| | TTGGCCTGCCTTCTGAAGCCTCCTTCTACGTCAATGTGCC | |
| | CTCTCGCATCGATCCTTCTGCTGCTCCCGACGGCAAGGAC | |
| | TCTGTCATTGTCTTGGTGCCTATTGGTCATATGAAGAGCA | |
| | AGACGGGCGATGCTTCCACCGAGAACTACCCGGCCATGGT | |
| | GGACAAGGCACGCAAGATGGTGCTGGCTGTGATTGAGCGT | |
| | CGTCTGGGCATGTCGAATTTCGCCGACTTGATTGAGCATG | |
| | AGCAAGTCAATGATCCCGCTGTATGGCAGAGCAAGTTCAA | |
| | TCTGTGGAGAGGCTCAATTCTGGGTTTGTCTCATGATGTG | |
| | CTTCAGGTGCTGTGGTTCCGTCCCAGCACAAAGGATTCTA | |
| | CCGGTCGTTATGATAACCTATTCTTTGTGGGTGCAAGCAC | |
| | GCATCCCGGAACTGGTGTTCCCATTGTCCTTGCAGGAAGC | |
| | AAGCTCACCTCTGACCAAGTIGTCAAGAGCTTTGGAAAGA | |
| | CGCCCAAGCCAAGAAAGATCGAGATGGAGAACACGCAAGC | |
| | ACCTTTGGAGGAGCCTGATGCTGAATCGACATTCCCTGTG | |
| | TGGTTCTGGTTGCGCGCTGCCTTTTGGGTCATGTTTATGT | |
| | TCTTTTACTTCTTCCCTCAATCCAATGGCCAAACGCCCGC | |
| | ATCTTTTATCAATAATTTGTTACCTGAAGTATTCCGCGTT | |
| | CATAACTCTAATGTCATTTAAACGCGCCACTACAAGTAGG | |
| | TACTACAAGTAGGACAGTATCCCCCCAAAATGTAACATAA | |
| | CACAAAATAAATGTATCTATACCTAATCGACATGGACTGC | |
| | ACCGCATTCGTTATCTTATCGTAGTCCTGCTTACTTTGAT | |
| | CCGTATCACATTCGTGTATCGCGATAAGAAGTAAACATTG | |
| | CTGCAAGTATAAATCAGGTCTCAAATCTCTTCTTGAAGAT | |
| | AGTTTTAAGCTCACCGTGAACTGAACGTGAACACTACGAG | |
| | TACGATTTTCAATATTATTACCGTCGATATAACAACCATG | |
| | CAATTTCTATATACATATATACATTCCCTCATTCGTCTGT | |
| | GCTGTNTGGTTCACTGCAGCAAATCAAACAGCTTTTCGTA | |
| | CTTTCTGTGCTGCGAATGTTTGTTACTATCCGCGTGCATT | |
| | CTTTGCAGCTTTNGAGATCCCGTATAGTCCTTCCACTAGC | |
| | TAGTAAGCTTGGTACCAGAGACGGGTTGGCGGCGTATTTG | |
| | TGTCCCAAAAAACAGCCCCAATTGCCCCAATTGACCCCAA | |
| | ATTGACCCAGTAGCGGGCCCAACCCCGGCGAGAGCCCCCT | |
| | TCACCCCACATATCAAACCTCCCCCGGTTCCCACACTTGC | |
| | CGTTAAGGGCGTAGGGTACTGCAGTCTGGAATCTACGCTT | |
| | GTTCAGACTTTGTACTAGTTTCTTTGTCTGGCCATCCGGG | |
| | TAACCCATGCCGGACGCAAAATAGACTACTGAAAATTTTT | |
| | TTGCTTTGTGGTTGGGACTTTAGCCAAGGGTATAAAAGAC | |
| | CACCGTCCCCGAATTACCTTTCCTCTTCTTTTCTCTCTCT | |
| | CCTTGTCAACTCACACCCGAAATCGTTAAGCATTTCCTTC | |
| | TGAGTATAAGAATCATTCGCTAGACACAAATGCTGCTCAC | |
| | CTACATGGAAGTCCACCTCTACTACACGCTGCCTGTGCTG | |
| | GGCGTCCTGTCCTGGCTGTCGCGGCCGTACTACACAGCCA | |
| | CCGATGCGCTCAAATTCAAATTTCTGACACTGGTTGCCTT | |
| | CACGACCGCCTCCGCCTGGGACAACTACATTGTCTACCAC | |
| | AAGGCGTGGTCCTACTGCCCCACCTGCGTCACCGCTGTCA | |
| | TTGGCTACGTGCCCTTGGAGGAGTACATGTTCTTCATCAT | |
| | CATGACTCTGTTGACCGTGGCATTCACCAATCTGGTGATG | |
| | CGCTGGCACCTGCACAGCTTCTTTATCAGGCCTGAAACGC | |
| | CCGTCATGCAGTCCGTCCTGGTCCGTCTTGTCCCCATAAC | |
| | AGCCTTATTAATCACTGCATACAAGGCTTGGCATTTGGCG | |
| | GTCCCTGGAAAGCCACTGTTCTACGGATCATGCATTTTGT | |
| | GGTACGCCTGTCCGGTTTTGGCCTTATTGTGGTTTGGTGC | |
| | TGGCGAGTACATGATGCGTCGTCCGCTGGCGGTGCTCGTC | |
| | TCCATTGCGCTGCCCACGCTGTTTCTCTGCTGGGTCGATG | |
| | TCGTCGCTATTGGCGCCGGCACATGGGACATTTCGCTGGC | |
| | CACAAGCACCGGCAAGTTCGTCGTGCCCCACCTGCCCGTG | |
| | GAGGAATTCATGTTCTTTGCGCTAATTAATACCGTTTTGG | |
| | TATTTGGTACGTGTGCGATCGATCGCACGATGGCGATCCT | |
| | CCACCTGTTCAAAAACAAGAGTCCTTATCAGCGCCCATAC | |
| | CAGCACAGCAAGTCGTTCCTCCACCAGATCCTCGAGATGA | |
| | CCTGGGCCTTCTGTTTACCCGACCAAGTGCTGCATTCAGA | |
| | CACATTCCACGACCTGTCCGTCAGCTGGGACATCCTGCGC | |
| | AAGGCCTCCAAGTCCTTTTACACGGCCTCTGCTGTCTTTC | |
| | CCGGCGACGTGCGCCAAGAGCTCGGTGTGCTATACGCCTT | |
| | TTGCAGAGCCACGGACGATCTCTGCGACAACGAGCAGGTC | |
| | CCTGTGCAGACGCGAAAGGAGCAGCTGATACTGACACATC | |
| | AGTTCGTCAGCGATCTGTTTGGCCAAAAGACAAGCGCGCC | |
| | GACTGCCATTGACTGGGACTTTTACAACGACCAACTGCCT | |
| | GCCTCGTGCATCTCTGCCTTCAAGTCGTTCACCCGTTTGC | |
| | GCCATGTGCTGGAAGCTGGAGCCATCAAGGAACTGCTCGA | |
| | CGGGTACAAGTGGGATTTGGAGCGTCGCTCCATCAGGGAT | |
| | CAGGAGGATCTCAGATATTACTCAGCTTGTGTCGCCAGCA | |
| | GTGTTGGTGAAATGTGCACTCGCATCATACTGGCCCACGC | |
| | CGACAAGCCCGCCTCCCGCCAGCAAACACAGTGGATCATT | |
| | CAGCGTGCGCGTGAAATGGGTCTGGTACTCCAATATACAA | |
| | ACATTGCAAGAGACATTGTCACCGACAGCGAGGAACTGGG | |
| | CAGATGCTACCTGCCTCAGGATTGGCTTACCGAGAAGGAG | |
| | GTGGCGCTGATTCAAGGCGGCCTTGCCCGAGAAATTGGCG | |
| | AGGAGCGATTGCTCTCACTGTCGCATCGCCTCATCTACCA | |
| | GGCAGACGAGCTCATGGTGGTTGCCAACAAGGGCATCGAC | |
| | AAGCTGCCCAGCCATTGTCAAGGCGGCGTGCGTGCGGCCT | |
| | GCAACGTCTATGCTTCCATTGGCACCAAGCTCAAGTCTTA | |
| | CAAGCACCACTATCCCAGCAGAGCACATGTCGGCAATTCG | |
| | AAACGAGTGGAAATTGCTCTTCTTAGCGTATACAACCTTT | |
| | ACACCGCGCCAATTGCGACTAGTAGTACCACACATTGCAG | |
| | ACAGGGAAAAATGAGAAATCTAAATACCATTTAAACGCGT | |
| | GCAATTAACAGATAGTTTGCCGGTGATAATTCTCTTAACC | |
| | TCCCACACTCCTTTGACATAACGATTTATGTAACGAAACT | |
| | GAAATTTGACCAGATATTGTTGTAAATAGAAAATCTGGCT | |
| | TGTAGGTGGCAAACTAGTAACGGCCGCCAGTGTGCTGGAA | |
| | TTGAATATTTACCGTTCGTATAATGTATGCTATACGAAGT | |
| | TATACCGGTCTCGTAGTGTTCACGTTCAGTTCACGGTGAG | |
| | CTTAAAACTATCTTCAAGAAGAGATTTGAGACCTGATTTA | |
| | TACTTGCAGCAATGTTTACTTCTTATCGCGATACACGAAT | |
| | GTGATACGGATCAAAGTAAGCAGGACTACGATAAGATAAC | |
| | GAATGCGGTGCAGTCCATGTCGATTAGGTATAGATACATT | |
| | TATTTTGTGTTATGTTACATTTTGGGGGGATACTGTCCTA | |
| | CTTGTAGTACCTACTTGTAGTGGCGCGTCTATTCCTTTGC | |
| | CCTCGGACGAGTGCTGGGGCGTCGGTTTCCACTATCGGCG | |
| | AGTACTTCTACACAGCCATCGGTCCAGACGGCCGCGCTTC | |
| | TGCGGGCGATTTGTGTACGCCCGACAGTCCCGGCTCCGGA | |
| | TCGGACGATTGCGTCGCATCGACCCTGCGCCCAAGCTGCA | |
| | TCATCGAAATTGCCGTCAACCAAGCTCTGATAGAGTTGGT | |
| | CAAGACCAATGCGGAGCATATACGCCCGGAGCCGCGGCGA | |
| | TCCTGCAAGCTCCGGATGCCTCCGCTCGAAGTAGCGCGTC | |
| | TGCTGCTCCATACAAGCCAACCACGGCCTCCAGAAGAAGA | |
| | TGTTGGCGACCTCGTATTGGGAATCCCCGAACATCGCCTC | |
| | GCTCCAGTCAATGACCGCTGTTATGCGGCCATTGTCCGTC | |
| | AGGACATTGTTGGAGCCGAAATCCGCGTGCACGAGGTGCC | |
| | GGACTTCGGGGCAGTCCTCGGCCCAAAGCATCAGCTCATC | |
| | GAGAGCCTGCGCGACGGACGCACTGACGGTGTCGTCCATC | |
| | ACAGTTTGCCAGTGATACACATGGGGATCAGCAATCGCGC | |
| | ATATGAAATCACGCCATGTAGTGTATTGACCGATTCCTTG | |
| | CGGTCCGAATGGGCCGAACCCGCTCGTCTGGCTAAGATCG | |
| | GCCGCAGCGATCGCATCCATGGCCTCCGCGACCGGCTGCA | |
| | GAACAGCGGGCAGTTCGGTTTCAGGCAGGTCTTGCAACGT | |
| | GACACCCTGTGCACGGCGGGAGATGCAATAGGTCAGGCTC | |
| | TCGCTGAATTCCCCAATGTCAAGCACTTCCGGAATCGGGA | |
| | GCGCGGCCGATGCAAAGTGCCGATAAACATAACGATCTTT | |
| | GTAGAAACCATCGGCGCAGCTATTTACCCGCAGGACATAT | |
| | CCACGCCCTCCTACATCGAAGCTGAAAGCACGAGATTCTT | |
| | CGCCCTCCGAGAGCTGCATCAGGTCGGAGACGCTGTCGAA | |
| | CTTTTCGATCAGAAACTTCTCGACAGACGTCGCGGTGAGT | |
| | TCAGGCTTTTTCATATGGGTACCTGAGAACATTTTTGTGT | |
| | CTAGGTGTTTGTGTTTGGACTGCGATCAGTGAAGAAAAGA | |
| | AGAGGAAAAATTGTGCAAGAAATTTTGCTTTCAAGACTTG | |
| | GCTGATGCAGCAGGGTAACTCTGGGACACAGACCTATGTT | |
| | TGTGGTTAAACTCAATGCACGTGGTACGTGCGTGGAGCGC | |
| | TTACCCATCCAAGGGTGTGGACATGGAACCGACGGTCCGT | |
| | GGAGTTGTGTAATGTCATTTTGGCGACTCTTGAAGCAAGG | |
| | CTATAAAAAAATTGTGTGGCTTGAGTCTTATCGAGCTCGG | |
| | TCACTACAAGAGTTAATCTTCCTGTCTCAGGCAGACAGGT | |
| | CAGGCAGGGTTACTTTTGGGTGTGCTGTAACTCACTGTAT | |
| | GGCCGTTAGTGCGCATAGACGTTGTACATACTGGACCGAA | |
| | TTGTAGCGTGCTCAATAGGGCCAATAAAGCTATTGTAGGG | |
| | ATCCATAACTTCGTATAATGTATGCTATACGAACGGTACC | |
| | CGGGCAATTCTGCAGATATCCATCACACTGGCGGCCGCTC | |
| | GAGCATGCATCTAGATGGCCTCCTTGGCCGGGTTTCAATT | |
| | CAATTCATCATTTTTTTTTTATTCTTTTTTTTGATTTCGG | |
| | TTTCTTTGAAATTTTTTTGATTCGGTAATCTCCGAACAGA | |
| | AGGAAGAACGAAGGAAGGAGCACAGACTTAGATTGGTATA | |
| | TATACGCATATGTAGTGTTGAAGAAACATGAAATTGCCCA | |
| | GTATTCTTAACCCAACTGCACAGAACAAAAACCTGCAGGA | |
| | AACGAAGATAAATCATGTCGAAAGCTACATATAAGGAACG | |
| | TGCTGCTACTCATCCTAGTCCTGTTGCTGCCAAGCTATTT | |
| | AATATCATGCACGAAAAGCAAACAAACTTGTGTGCTTCAT | |
| | TGGATGTTCGTACCACCAAGGAATTACTGGAGTTAGTTGA | |
| | AGCATTAGGTCCCAAAATTTGTTTACTAAAAACACATGTG | |
| | GATATCTTGACTGATTTTTCCATGGAGGGCACAGTTAAGC | |
| | CGCTAAAGGCATTATCCGCCAAGTACAATTTTTTACTCTT | |
| | CGAAGACAGAAAATTTGCTGACATTGGTAATACAGTCAAA | |
| | TTGCAGTACTCTGCGGGTGTATACAGAATAGCAGAATGGG | |
| | CAGACATTACGAATGCACACGGTGTGGTGGGCCCAGGTAT | |
| | TGTTAGCGGTTTGAAGCAGGCGGCAGAAGAAGTAACAAAG | |
| | GAACCTAGAGGCCTTTTGATGTTAGCAGAATTGTCATGCA | |
| | AGGGCTCCCTATCTACTGGAGAATATACTAAGGGTACTGT | |
| | TGACATTGCGAAGAGCGACAAAGATTTTGTTATCGGCTTT | |
| | ATTGCTCAAAGAGACATGGGTGGAAGAGATGAAGGTTACG | |
| | ATTGGTTGATTATGACACCCGGTGTGGGTTTAGATGACAA | |
| | GGGAGACGCATTGGGTCAACAGTATAGAACCGTGGATGAT | |
| | GTGGTCTCTACAGGATCTGACATTATTATTGTTGGAAGAG | |
| | GACTATTTGCAAAGGGAAGGGATGCTAAGGTAGAGGGTGA | |
| | ACGTTACAGAAAAGCAGGCTGGGAAGCATATTTGAGAAGA | |
| | TGCGGCCAGCAAAACTAAAAAACTGTATTATAAGTAAATG | |
| | CATGTATACTAAACTCACAAATTAGAGCTTCAATTTAATT | |
| | ATATCAGTTATTACCCGGGAATCTCGGTCGTAATGATTTT | |
| | TATAATGACGAAAAAAAAAAAATTGGAAAGAAAACCCCCC | |
| | CCCCGCAGCGTTGGGTCCTGGCCACGGGTGCGCATGATCG | |
| | TGCTCCTGTCGTTGAGGACCCGGCTAGGCTGGCGGGGTTG | |
| | CCTTACTGGTTAGCAGAATGAATCACCGATACGCGAGCGA | |
| | ACGTGAAGCGACTGCTGCTGCAAAACGTCTGCGACCTGAG | |
| | CAACAACATGAATGGTCTTCGGTTTCCGTGTTTCGTAAAG | |
| | TCTGGAAACGCGGAAGTCAGCGCCCTGCACCATTATGTTC | |
| | CGGATCTGCATCGCAGGATGCTGCTGGCTACCCTGTGGAA | |
| | CACCTACATCTGTATTAACGAAGCGCTGGCATTGACCCTG | |
| | AGTGATTTTTC | |
| MB9 | GGTTCCGCTTCCTCGCTCACTGACTCGCTGCGCTCGGTCG | 5 |
| 282 | TTCGGCTGCGGCGAGCGGTATCAGCTCACTCAAAGGCGGT | |
| | AATACGGTTATCCACAGAATCAGGGGATAACGCAGGAAAG | |
| | AACATGTGAGCAAAAGGCCAGCAAAAGGCCAGGAACCGTA | |
| | AAAAGGCCGCGTTGCTGGCGTTTTTCCATAGGCTCCGCCC | |
| | CCCTGACGAGCATCACAAAAATCGACGCTCAAGTCAGAGG | |
| | TGGCGAAACCCGACAGGACTATAAAGATACCAGGCGTTTC | |
| | CCCCTGGAAGCTCCCTCGTGCGCTCTCCTGTTCCGACCCT | |
| | GCCGCTTACCGGATACCTGTCCGCCTTTCTCCCTTCGGGA | |
| | AGCGTGGCGCTTTCTCATAGCTCACGCTGTAGGTATCTCA | |
| | GTTCGGTGTAGGTCGTTCGCTCCAAGCTGGGCTGTGTGCA | |
| | CGAACCCCCCGTTCAGCCCGACCGCTGCGCCTTATCCGGT | |
| | AACTATCGTCTTGAGTCCAACCCGGTAAGACACGACTTAT | |
| | CGCCACTGGCAGCAGCCACTGGTAACAGGATTAGCAGAGC | |
| | GAGGTATGTAGGCGGTGCTACAGAGTTCTTGAAGTGGTGG | |
| | CCTAACTACGGCTACACTAGAAGGACAGTATTTGGTATCT | |
| | GCGCTCTGCTGAAGCCAGTTACCTTCGGAAAAAGAGTTGG | |
| | TAGCTCTTGATCCGGCAAACAAACCACCGCTGGTAGCGGT | |
| | GGTTTTTTTGTTTGCAAGCAGCAGATTACGCGCAGAAAAA | |
| | AAGGATCTCAAGAAGATCCTTTGATCTTTTCTACGGGGTC | |
| | TGACGCTCAGTGGAACGAAAACTCACGTTAAGGGATTTTG | |
| | GTCATGAGATTATCAAAAAGGATCTTCACCTAGATCCTTT | |
| | TAAATTAAAAATGAAGTTTTAAATCAATCTAAAGTATATA | |
| | TGAGTAAACTTGGTCTGACAGTTACCAATGCTTAATCAGT | |
| | GAGGCACCTATCTCAGCGATCTGTCTATTTCGTTCATCCA | |
| | TAGTTGCCTGACTCCCCGTCGTGTAGATAACTACGATACG | |
| | GGAGGGCTTACCATCTGGCCCCAGTGCTGCAATGATACCG | |
| | CGAGACCCACGCTCACCGGCTCCAGATTTATCAGCAATAA | |
| | ACCAGCCAGCCGGAAGGGCCGAGCGCAGAAGTGGTCCTGC | |
| | AACTTTATCCGCCTCCATCCAGTCTATTAATTGTTGCCGG | |
| | GAAGCTAGAGTAAGTAGTTCGCCAGTTAATAGTTTGCGCA | |
| | ACGTTGTTGCCATTGCTGCAGGCATCGTGGTGTCACGCTC | |
| | GTCGTTTGGTATGGCTTCATTCAGCTCCGGTTCCCAACGA | |
| | TCAAGGCGAGTTACATGATCCCCCATGTTGTGCAAAAAAG | |
| | CGGTTAGCTCCTTCGGTCCTCCGATCGTTGTCAGAAGTAA | |
| | GTTGGCCGCAGTGTTATCACTCATGGTTATGGCAGCACTG | |
| | CATAATTCTCTTACTGTCATGCCATCCGTAAGATGCTTTT | |
| | CTGTGACTGGTGAGTACTCAACCAAGTCATTCTGAGAATA | |
| | GTGTATGCGGCGACCGAGTTGCTCTTGCCCGGCGTCAACA | |
| | CGGGATAATACCGCGCCACATAGCAGAACTTTAAAAGTGC | |
| | TCATCATTGGAAAACGTTCTTCGGGGCGAAAACTCTCAAG | |
| | GATCTTACCGCTGTTGAGATCCAGTTCGATGTAACCCACT | |
| | CGTGCACCCAACTGATCTTCAGCATCTTTTACTTTCACCA | |
| | GCGTTTCTGGGTGAGCAAAAACAGGAAGGCAAAATGCCGC | |
| | AAAAAAGGGAATAAGGGCGACACGGAAATGTTGAATACTC | |
| | ATACTCTTCCTTTTTCAATATTATTGAAGCATTTATCAGG | |
| | GTTATTGTCTCATGAGCGGATACATATTTGAATGTATTTA | |
| | GAAAAATAAACAAATAGGGGTTCCGCGCACATTTCCCCGA | |
| | AAAGTGCCACCTGACGTCTAAGAAACCATTATTATCATGA | |
| | CATTAACCTATAAAAATAGGCGTATCACGAGGCCCTTTCG | |
| | TCTGGCCTAGGAAGCGACTTCCAATCGCTTTGCATATCCA | |
| | GTACCACACCCACAGGCGTTTGTGCTACTCTACTGATAGC | |
| | AATAGATGCGTCATAATTGGTTGGCCCGCTGAGCCTCCAC | |
| | AGGATACTATTGCACATACCCTGGTCATGTGCAGATCAGC | |
| | TCATTTGTGGAGACTCTGGAGTAACTTAGACGACGCCTGG | |
| | TTCAATTGCCGCAATGTGCGCCCACGCAGATAATGTATTG | |
| | AGGGGTGGAGCGCCTCTTGGGGACTTGCTGTACTTGTACG | |
| | GGATATTAAACGCACTCAGCAAGACCATGACGTAAAACAC | |
| | ACCTACTGTACGATACGTACTGTAGGTATTGTACTCGTAC | |
| | CCGGTACTACAAATAGTACGATACTATACGGAGTGTATTT | |
| | GTACCTTGATATACGACTGGCGGAGTGAAGAGAAGGAGTT | |
| | GAACAAGACCAGATGGGGATATCAGCCCCAGTGCTTTGTA | |
| | TTACAAGTACGAGTACTTAATAGATACTGTAAGGCTATTG | |
| | ATACGGATGGCAGTAAGTCATTGAGTAAGCAATTGTGGCC | |
| | CAGCATCTCCCCTACGTACTTGTACCATACCCCATGGAGA | |
| | CACCAATGGTCTTTCACGCACACTGTCGTGTGCTGTATCG | |
| | CAGAATCGGGTGTCCAACCAAATGCCGTTACCCCCACGTC | |
| | ACAGCCGATAGACAGATACACCATCAATACCAGCAGGTTG | |
| | TATCATGCGGTTGGCTGAAGGTAAGCTGATTGGTCTAAAA | |
| | ACTGTAGCTGTCCTAATTCAACGAGCGCTATTTGGGGCCA | |
| | ACCACCTCGGCCAAGCGGCCTTTAATCTGCGTGCCCCAGA | |
| | GGCGTCTAATGAGGCTCTGGCCGCCACTGTAGGAGTGTTT | |
| | CTCTGTGCGCACACGCAGTTTTGAGTTTGGGCGACTTTCC | |
| | CTTTTTCCCAATTGCGTACACACACAGCTCCGAGCTAAGC | |
| | GCTGTCCTTGAACCTTCTCCCTCTTTTCCCTCTTTTTCTC | |
| | TTCCCCTTCCCCTCCTCCACATTAAGGCCAAATCCTGAAT | |
| | TGCACCAACTAGTACAACGACAACAATGGACAAGAAGTAC | |
| | TCCATCGGTTTGGACATTGGTACTAACTCTGTCGGCTGGG | |
| | CCGTCATCACCGACGAGTACAAGGTTCCCTCCAAGAAGTT | |
| | CAAGGTCCTTGGCAACACCGACCGACACTCTATCAAGAAG | |
| | AACCTGATCGGTGCTCTGCTGTTCGACTCTGGCGAGACTG | |
| | CCGAGGCCACCCGACTGAAGCGAACCGCTCGACGCCGATA | |
| | CACCCGACGAAAGAACCGAATCTGTTACCTCCAGGAGATC | |
| | TTCAGCAACGAGATGGCTAAGGTCGACGACTCCTTCTTCC | |
| | ACCGACTCGAGGAGTCTTTCCTGGTCGAAGAGGATAAGAA | |
| | GCACGAGCGACACCCCATCTTCGGCAACATTGTTGATGAG | |
| | GTTGCCTACCATGAGAAGTACCCCACCATCTACCACCTCC | |
| | GAAAGAAGCTCGTCGACTCCACTGACAAGGCTGACCTCCG | |
| | ACTCATCTACCTTGCTCTCGCCCACATGATCAAGTTCCGA | |
| | GGTCACTTCCTCATTGAGGGTGATCTCAACCCCGACAACT | |
| | CCGACGTTGACAAGCTGTTCATCCAGCTCGTCCAGACCTA | |
| | CAACCAGCTCTTTGAGGAGAACCCTATCAACGCTTCTGGT | |
| | GTTGACGCCAAGGCCATTCTCTCCGCCCGACTCTCTAAGT | |
| | CCCGACGACTCGAGAACCTCATTGCCCAGCTGCCCGGCGA | |
| | GAAGAAGAACGGCCTCTTCGGTAACCTGATTGCTCTCTCT | |
| | CTTGGTCTGACCCCCAACTTCAAGTCCAACTTTGACCTCG | |
| | CCGAGGACGCCAAGCTCCAGCTGTCCAAGGACACCTACGA | |
| | TGACGATCTGGACAACCTCCTGGCCCAGATCGGTGACCAG | |
| | TACGCCGATCTCTTCCTTGCCGCCAAGAACCTCTCCGACG | |
| | CCATCCTGCTCTCCGACATCCTCCGAGTCAACACCGAGAT | |
| | TACCAAGGCTCCTCTGTCTGCCTCTATGATCAAGCGATAC | |
| | GACGAGCACCACCAGGATCTCACTCTTCTCAAGGCTCTCG | |
| | TCCGACAGCAGCTCCCCGAGAAGTACAAGGAGATTTTCTT | |
| | TGACCAGTCCAAGAACGGTTACGCTGGCTACATTGACGGT | |
| | GGTGCTTCCCAGGAAGAGTTTTACAAGTTCATCAAGCCTA | |
| | TTCTGGAGAAGATGGACGGTACCGAGGAGCTGCTCGTCAA | |
| | GCTCAACCGAGAGGACCTCCTTCGAAAGCAGCGAACCTTC | |
| | GATAACGGCTCCATCCCCCACCAGATCCACCTGGGTGAGC | |
| | TCCACGCCATTCTCCGAAGACAAGAGGACTTCTACCCCTT | |
| | CCTAAAGGATAACCGAGAGAAGATCGAGAAGATTCTCACC | |
| | TTCCGAATCCCCTACTACGTCGGTCCCCTCGCTCGAGGTA | |
| | ACTCCCGATTTGCTTGGATGACCCGAAAGTCCGAGGAGAC | |
| | TATCACCCCCTGGAACTTTGAAGAGGTAGTCGACAAGGGT | |
| | GCCTCCGCCCAGTCTTTCATTGAGCGGATGACCAACTTCG | |
| | ATAAGAACCTCCCCAACGAGAAGGTCCTTCCCAAGCACTC | |
| | TCTCCTCTACGAGTACTTCACCGTCTACAACGAGCTGACC | |
| | AAGGTCAAGTACGTTACCGAGGGCATGCGAAAGCCCGCTT | |
| | TCCTCTCTGGTGAGCAGAAGAAGGCCATTGTCGACCTCCT | |
| | GTTCAAGACTAACCGAAAAGTCACCGTCAAGCAGCTCAAG | |
| | GAAGACTACTTCAAGAAGATTGAGTGCTTCGACTCCGTCG | |
| | AGATTTCCGGTGTCGAGGACCGATTCAACGCCTCCCTCGG | |
| | CACCTACCACGATCTTCTGAAGATCATCAAGGACAAGGAC | |
| | TTTCTTGATAACGAGGAGAACGAGGACATTCTCGAGGACA | |
| | TCGTCCTCACCCTCACCCTTTTCGAGGATCGAGAGATGAT | |
| | CGAGGAGCGACTCAAGACCTACGCCCATCTCTTCGACGAC | |
| | AAGGTCATGAAGCAACTCAAGCGACGACGATACACTGGCT | |
| | GGGGCCGACTTTCCCGAAAGCTCATCAACGGCATCCGAGA | |
| | CAAGCAGTCTGGCAAGACCATCCTGGACTTCCTGAAGTCC | |
| | GACGGTTTCGCCAACCGAAACTTCATGCAGCTCATCCACG | |
| | ACGACTCTCTTACCTTCAAAGAGGATATCCAGAAGGCCCA | |
| | GGTTTCTGGCCAGGGCGACTCCCTCCACGAGCACATTGCC | |
| | AACCTCGCCGGATCCCCCGCCATCAAAAAGGGTATCCTCC | |
| | AGACCGTCAAGGTTGTCGACGAACTCGTGAAGGTCATGGG | |
| | CCGACACAAGCCCGAGAACATCGTTATCGAGATGGCCCGA | |
| | GAGAACCAGACCACCCAGAAGGGTCAGAAGAACTCCCGAG | |
| | AGCGAATGAAGCGAATCGAAGAGGGTATCAAGGAGCTCGG | |
| | TTCCCAGATTCTCAAGGAGCACCCCGTCGAGAACACCCAG | |
| | CTCCAGAACGAGAAACTCTACCTGTACTACCTCCAGAATG | |
| | GCCGAGACATGTACGTTGACCAGGAGCTCGACATCAACCG | |
| | ACTCTCCGACTACGACGTCGACCACATTGTTCCTCAGTCC | |
| | TTCCTCAAGGACGACTCCATCGACAACAAGGTTCTGACCC | |
| | GATCTGACAAGAACCGAGGTAAGTCCGACAACGTTCCCTC | |
| | CGAAGAGGTCGTTAAGAAGATGAAGAACTACTGGCGACAG | |
| | CTTCTCAACGCCAAACTGATCACCCAGCGAAAGTTTGACA | |
| | ACCTCACCAAGGCCGAGCGAGGTGGTCTGTCCGAGCTGGA | |
| | CAAGGCCGGCTTCATTAAGCGACAGCTGGTCGAGACTCGA | |
| | CAGATCACCAAGCACGTCGCCCAGATCCTCGACTCCCGAA | |
| | TGAACACCAAGTACGACGAGAACGACAAGCTCATCCGGGA | |
| | GGTCAAGGTCATCACCCTGAAGTCTAAGCTTGTCTCCGAC | |
| | TTCCGAAAGGACTTCCAGTTCTACAAGGTCCGAGAGATCA | |
| | ACAACTACCACCACGCCCACGACGCCTACCTCAACGCCGT | |
| | TGTTGGTACCGCCCTCATCAAGAAGTATCCCAAGCTCGAG | |
| | TCCGAGTTCGTTTACGGCGACTACAAGGTTTACGATGTCC | |
| | GAAAGATGATTGCCAAGTCCGAGCAGGAGATCGGTAAGGC | |
| | CACCGCCAAGTACTTTTTCTACTCCAACATCATGAATTTC | |
| | TTCAAGACCGAGATCACTCTCGCCAACGGTGAGATTCGAA | |
| | AGCGACCCCTGATTGAGACTAATGGTGAGACTGGTGAGAT | |
| | CGTCTGGGATAAGGGCCGAGACTTCGCCACCGTCCGAAAG | |
| | GTCCTGTCCATGCCCCAGGTCAACATTGTCAAGAAGACCG | |
| | AGGTCCAGACCGGTGGCTTCTCCAAGGAGTCCATTCTCCC | |
| | CAAGCGAAACTCCGACAAACTCATCGCCCGTAAGAAGGAC | |
| | TGGGATCCGAAGAAGTACGGTGGTTTCGATTCTCCCACCG | |
| | TTGCCTACTCCGTCCTCGTTGTTGCTAAAGTCGAGAAGGG | |
| | TAAGTCTAAGAAACTCAAGTCCGTGAAGGAGCTACTCGGT | |
| | ATCACCATCATGGAGCGATCTTCTTTTGAGAAGAACCCCA | |
| | TTGACTTCCTCGAGGCCAAGGGTTACAAAGAGGTCAAGAA | |
| | GGACCTGATTATCAAGCTGCCCAAGTACTCCCTCTTTGAG | |
| | CTCGAGAACGGCCGAAAGCGAATGCTGGCTTCCGCTGGTG | |
| | AGCTGCAGAAGGGCAACGAGCTCGCTCTGCCCTCCAAGTA | |
| | CGTCAACTTCCTCTACCTGGCCTCCCACTACGAGAAGCTC | |
| | AAGGGCTCCCCCGAGGACAACGAGCAGAAGCAGCTGTTCG | |
| | TTGAGCAGCACAAGCACTACCTCGACGAGATCATCGAGCA | |
| | GATCTCCGAGTTCTCCAAGCGAGTCATCCTCGCTGACGCC | |
| | AACCTTGATAAGGTTCTCTCTGCTTACAACAAGCACCGGG | |
| | ACAAGCCCATCCGAGAGCAGGCCGAGAATATCATCCACCT | |
| | CTTCACTCTCACCAACCTCGGCGCTCCTGCTGCCTTCAAG | |
| | TACTTCGACACCACCATTGACCGAAAGAGGTACACCTCCA | |
| | CCAAGGAAGTCCTCGACGCCACCCTGATCCACCAGTCCAT | |
| | CACCGGCCTCTACGAAACCCGAATCGACCTCTCCCAGCTC | |
| | GGCGGTGACTCTCGAGCCGACCCCAAGAAGAAGCGAAAAG | |
| | TCTAAATATCCGAAGATCAAGAGCGAAGCAAGTTGTAAGT | |
| | CCAGGACATGTTTCCCGCCCACGCGAGTGATTTATAACAC | |
| | CTCTCTTTTTTGACACCCGCTCGCCTTGAAATTCATGTCA | |
| | CATAAATTATAGTCAACGACGTTTGAATAACTTGTCTTGT | |
| | AGTTCGATGATGATCATATGATTACATTAATAGTAATTAC | |
| | TGTATTTGATATATATACTAATTACAATAGTACATATTAG | |
| | AACATACAATAGTTAGTGCCGTGAAGTGGCTTAAAATACC | |
| | GCGAGTCGATTACGTAATATTATTACCTCTTGCCCATCGA | |
| | ACGTACAAGTACTCCTCTGTTCTCTCCTTCCTTTGCTTTG | |
| | TGCACGAAGAACTGCGGTCAGGTGACACAACTTTTTCCAT | |
| | CTCAGGGTGTGTCGCGTGTGCTTCATCCAAACTTTAGTTG | |
| | GGGTTCGGGTTCGCGCGAGATGATCACGTGCCCTGATTTG | |
| | GTGTCGTCCCCCGTCGCGCTGCGCACGTGATTTATTTATT | |
| | TCCGGTGGCTGCTGTCTACGCGGGGCCTTCTCTGCCCTTC | |
| | TGTTTCAACCTTCGGGCGGTTCTCGTAACCAGCAGTAGCA | |
| | ATCCATTTCGAAACTCAAAGAGCTAAAAACGTTAAACCTC | |
| | AGCAGTCGCTCGACGAATGGGCTGCGGTTGGGAAGCCCAC | |
| | GAGGCCTATAGCCAGAGCCTCGAGTTGACAGGAGCCCAGA | |
| | CGCCTTTTCCAACGGCAACTTTTATATAAAATGGCAATGT | |
| | ATTCATGCAATTGCGGCCGTGTCAGGTTGGAGACACTGGA | |
| | CCACACTCTCCATTGCTTCCTGAGGAGATGGATCATTGCT | |
| | AGTGCATCTACGCGCAGCAATCCCGCAAGCTCGACAACCG | |
| | TAGATGGGCTTTGGTGGGCCAATCAATTACGCAACCCGCA | |
| | CGTTAAATTGTATGAGGAAGGAAGGCCACGGTACAAAGTG | |
| | GGTGGTCTTCACCCAGTGGTTGTTGGTGGCGTCATGCAGA | |
| | CCATGCATTGGGGATAGCACAGGGTTGGGGTGTCTTGTGG | |
| | ACTCAATGGGTGAAAGGAGATGGAAAAGGGCGGTGAAAAG | |
| | TGGTAGAATCGAAATCCCTGACGTCAATTTATAAAGTAAA | |
| | ATGCGTTTCTGCCATTTTGCTCCCCTCCTTCTTTCGCAAT | |
| | CGCCTCCCCAAAAGTTGTCGTGGCAGTACACATGCTTGCA | |
| | TACAATGAAGCTAATCCGGCTTGCTCAGTAGTTGCTATAT | |
| | CCAGGCATGGTGTGAAACCCCTCAAAGTATATATAGGAGC | |
| | GGTGAGCCCCAGTCTGGGGTCTTTTCTCTCCATCTCAAAA | |
| | CTACTTTCTCACAATGACGAGTCTGATGAGTCCGTGAGGA | |
| | CGAAACGAGTAAGCTCGTCACTCGTCTACGCATGCCTGGG | |
| | TTTTAGAGCTAGAAATAGCAAGTTAAAATAAGGCTAGTCC | |
| | GTTATCAACTTGAAAAAGTGGCACCGAGTCGGTGCTTTTG | |
| | GCCGGCATGGTCCCAGCCTCCTCGCTGGCGCCGGCTGGGC | |
| | AACATGCTTCGGCATGGCGAATGGGACTAAACTTCGAGCT | |
| | AATCCAGTAGCTTACGTTACCCAGGGGCAGGTCAACTGGC | |
| | TAGCCACGAGTCTGTCCCAGGTCGCAATTTAGTGTAATAA | |
| | ACAATATATATATTGAGTCTAAAGGGAATTGTAGCTATTG | |
| | TGATTGTGTGATTTTCGTCTTGCTGGTTCTTATTGTGTCC | |
| | CATTCGTTTCATCCTGATGAGGACCCCTGGAACCGGTGTT | |
| | TTCTTAGTCTCTGCAATCGCTAGTCTTGTTGCTATGACAG | |
| | TTGCGTCGACACTATTCAGGTCATCTATCGGTTATTCTGA | |
| | TATTATAATACCTCCGGATCGATGTACCTGATTTATACTT | |
| | GCAGCAATGTTTACTTCTTATCGTTGGACCCCGTCTTCAA | |
| | TTACACTTCCCAACTGGGAACACCCCTCTTTATCGACCCA | |
| | TTTTAGGTAATTTACCCTAGCCCATTGTCTCCATAAGGAA | |
| | TATTACCCTAACCCACAGTCCAGGGTGCCCAGGTCCTTCT | |
| | TTGGCCAAATTTTAACTTCGGTCCTATGGCACAGCGGTAG | |
| | CGCGTGAGATTGCAAATCTTAAGGTCCCGAGTTCGAATCT | |
| | CGGTGGGACCTAGTTGAAAAATACCTCTAATGCGCCGATG | |
| | GTTTAGTGGTAAAATCCATCGTTGCCATCGATGGGCCCCC | |
| | GGTTCGATTCCGGGTCGGCGCAGGTTGACGTCGAACGTCT | |
| | TGTCTTCCGAAGTTTTAGAGCTAGAAATAGCAAGTTAAAA | |
| | TAAGGCTAGTCCGTTATCAACTTGAAAAAGTGGCACCGAG | |
| | TCGGTGCTTTTTTTTTTCGCGATACACGAATGTGATACGG | |
| | ATCAAAGTAAGCAGGACTACGATAAGATAACGAATGCGGT | |
| | GCAGTCCATGTCGATTAGGTATAGATACATTTATTTTGTG | |
| | TTATGTTACATTTTGGGGGGATACTGTCCTACTTGTAGTA | |
| | CCTACTTGTAGTGGCGCGTCTATTCCTTTGCCCTCGGACG | |
| | AGTGCTGGGGCGTCGGTTTCCACTATCGGCGAGTACTTCT | |
| | ACACAGCCATCGGTCCAGACGGCCGCGCTTCTGCGGGCGA | |
| | TTTGTGTACGCCCGACAGTCCCGGCTCCGGATCGGACGAT | |
| | TGCGTCGCATCGACCCTGCGCCCAAGCTGCATCATCGAAA | |
| | TTGCCGTCAACCAAGCTCTGATAGAGTTGGTCAAGACCAA | |
| | TGCGGAGCATATACGCCCGGAGCCGCGGCGATCCTGCAAG | |
| | CTCCGGATGCCTCCGCTCGAAGTAGCGCGTCTGCTGCTCC | |
| | ATACAAGCCAACCACGGCCTCCAGAAGAAGATGTTGGCGA | |
| | CCTCGTATTGGGAATCCCCGAACATCGCCTCGCTCCAGTC | |
| | AATGACCGCTGTTATGCGGCCATTGTCCGTCAGGACATTG | |
| | TTGGAGCCGAAATCCGCGTGCACGAGGTGCCGGACTTCGG | |
| | GGCAGTCCTCGGCCCAAAGCATCAGCTCATCGAGAGCCTG | |
| | CGCGACGGACGCACTGACGGTGTCGTCCATCACAGTTTGC | |
| | CAGTGATACACATGGGGATCAGCAATCGCGCATATGAAAT | |
| | CACGCCATGTAGTGTATTGACCGATTCCTTGCGGTCCGAA | |
| | TGGGCCGAACCCGCTCGTCTGGCTAAGATCGGCCGCAGCG | |
| | ATCGCATCCATGGCCTCCGCGACCGGCTGCAGAACAGCGG | |
| | GCAGTTCGGTTTCAGGCAGGTCTTGCAACGTGACACCCTG | |
| | TGCACGGCGGGAGATGCAATAGGTCAGGCTCTCGCTGAAT | |
| | TCCCCAATGTCAAGCACTTCCGGAATCGGGAGCGCGGCCG | |
| | ATGCAAAGTGCCGATAAACATAACGATCTTTGTAGAAACC | |
| | ATCGGCGCAGCTATTTACCCGCAGGACATATCCACGCCCT | |
| | CCTACATCGAAGCTGAAAGCACGAGATTCTTCGCCCTCCG | |
| | AGAGCTGCATCAGGTCGGAGACGCTGTCGAACTTTTCGAT | |
| | CAGAAACTTCTCGACAGACGTCGCGGTGAGTTCAGGCTTT | |
| | TTCATATGGGTACCTGAGAACATTTTTGTGTCTAGGTGTT | |
| | TGTGTTTGGACTGCGATCAGTGAAGAAAAGAAGAGGAAAA | |
| | ATTGTGCAAGAAATTTTGCTTTCAAGACTTGGCTGATGCA | |
| | GCAGGGTAACTCTGGGACACAGACCTATGTTTGTGGTTAA | |
| | ACTCAATGCACGTGGTACGTGCGTGGAGCGCTTACCCATC | |
| | CAAGGGTGTGGACATGGAACCGACGGTCCGTGGAGTTGTG | |
| | TAATGTCATTTTGGCGACTCTTGAAGCAAGGCTATAAAAA | |
| | AATTGTGTGGCTTGAGTCTTATCGAGCTCGGTCACTACAA | |
| | GAGTTAATCTTCCTGTCTCAGGCAGACAGGTCAGGCAGGG | |
| | TTACTTTTGGGTGTGCTGTAACTCACTGTATGGCCGTTAG | |
| | TGCGCATAGACGTTGTACATACTGGACCGAATTGTAGCGT | |
| | GCTCAATAGGGCCAATAAAGCTATTGTAGGGATCCGAATT | |
| | TTCAGAACCTAATTTATCTGTTACCCGGCCTGTGGCTCGC | |
| | ACAGCTTAAAAATGGTCAAACTTTCCCCTTCTTGTCTTTT | |
| | TTTCCTCACATTCATCAGGTTCTTGTCTTGATCTTTCAAG | |
| | TGAGTATTAATTACCGACCTTGGTTCTTCATTGGGAGAGC | |
| | ATTGGAAGCCGTGGTGCAGCAACCACAAAACGGTTCTTCC | |
| | CCTTCGATACCTTCTTGCCTGCCTTTCAATACAAGTCGGC | |
| | TCGATTAGCGGTGGTCGCCCCCGCCAGCGGAGAACATGGA | |
| | ACTAACCCAGAATGAGAGCTAAGTGGAGAAAGAAGAGAGT | |
| | CAGACGACTCAAGCGAAAGCGCCGCAAGGTCCGAGCTCGA | |
| | TCCAAATAAGCGGTTTTTAACGGAGATTTAACACTAAATC | |
| | GAAGAACTTTTCCCGTTTCATTTGCGAATGAGCTCGTTAA | |
| | CAAAATCCCCCAGTTTTTTTATCCAGCTGTAAGGATTGAC | |
| | ATTAGTAATGAATTATTGTTTGGTATATTTAAATCTGTAG | |
| | TTCCTTTCTGTCCGTGTCGGCAACTGTCGTACTCGTGATT | |
| | TACTTGTATTGACGAATACTTACTGTAGCGCACTCTGCTG | |
| | CTACTGGTCGTAAGGATGTGCTATTTCGGTGTATGGTGGG | |
| | TTTTTTGGGGGTCGGAACCGAAGACTGTTACACGGGCACG | |
| | GCTCGTTGTGTACACGCACAGAGCTCTTGCGAGTCATGTT | |
| | GTAGCTAGCTCGTCGTGTTCAGGAACTGTTCGATGGTTCG | |
| | GAGAGAGTCGCCGCCCAGAACATACGCGCACCGATGTCAG | |
| | CAGACAGCCTTATTACAAGTATATTCAAGCAAGTATATCC | |
| | GTAGGGTGCGGGTGATTTGGATCTAAGGTTCGTACTCAAC | |
| | ACTCACGAGCAGCTTGCCTATGTTACATCCTTTTATCAGA | |
| | CATAACATAATTGGAGTTTACTTACACACGGGGTGTACCT | |
| | GTATGAGCACCACCTACAATTGTAGCACTGGTACTTGTAC | |
| | AAAGAATTTATTCGTACGAATCACAGGGACGGCCGCCCTC | |
| | ACCGAACCAGCGAATACCTCAGCGGTCCCCTGCAGTGACT | |
| | CAACAAAGCGATATGAACATCTTGCGATGGTATCCTGCTG | |
| | ATAGTTTTTACTGTACAAACACCTGTGTAGCTCCTTCTAG | |
| | CATTTTTAAGTTATTCACACCTCAAGGGGAGGGATAAATT | |
| | AAATAAATTCCAAAAGCGAAGATCGAGAAACTAAATTAAA | |
| | ATTCCAAAAACGAAGTTGGAACACAACCCCCCGAAAAAAA | |
| | ACAACAAACAAAAAACCCAACAAAATAAACAAAAACAAAA | |
| | TAAATATATAACTACCAGTATCTGACTAAAAGTTCAAATA | |
| | CTCGTACTTACAACAAATAGAAATGAGCCGGCCAAAATTC | |
| | TGCAGAAAAAAATTTCAAACAAGTACTGGTATAATTAAAT | |
| | TAAAAAACACATCAAAGTATCATAACGTTAGTTATTTTAT | |
| | TTTATTTAATAAAAGAAAACAACAAGATGGGCTCAAAACT | |
| | TTCAACTTATACGATACATACCAAATAACAATTTAGTATT | |
| | TATCTAAGTGCTTTTCGTAGATAATGGAATACAAATGGAT | |
| | ATCCAGAGTATACACATGGATAGTATACACTGACACGACA | |
| | ATTCTGTATCTCTTTATGTTAACTACTGTGAGGCATTAAA | |
| | TAGAGCTTGATATATAAAATGTTACATTTCACAGTCTGAA | |
| | CTTTTGCAGATTACCTAATTTGGTAAGATATTAATTATGA | |
| | ACTGAAAGTTGATGGCATCCCTAAATTTGATGAAAGATGA | |
| | AATTGTAAATGAGGTGGTAAAAGAGCTACAGTCGTTTTGT | |
| | TTTGAGATACCATCATCTCTAACGAAATATCTATTAAAAA | |
| | TCTCAGTGTGATCATGAGTCATTGCCATCCTGGAAAATGT | |
| | CATCATGGCTGATATTTCTAACTGTTTACTTGAGATAAAT | |
| | ATATATTTACAAGAACTTCCCTTGAAATTAATTTAGATAT | |
| | AAAATGTTTGCGGGCAAGTTACTACGAGGAATAAATTATA | |
| | TCTAGA | |
| DNA | AGGCCAAGAGACTGTGAGGCCCATGTCCACTTGGTCACGG | 6 |
| 41 | GTTGACAAAAAGAAACGCCTCCTTCCCTGTGTTTGTCCAC | |
| | GATGGCCAGAATACCACGCAGTGCTAGAATATTTCTGTAT | |
| | TTTTGAAATATCAATTTTGACCACGTCCGACTCCAAATAG | |
| | CACCATATAACCAACCCCTGGTTTAGACCCACACACACTA | |
| | CTTACCACCTGCAGCACATCACGTCGACATCCCCGCGATC | |
| | AATCGCATCAGAAACCCTAGCCCTATTTCGGGAGGTCAGC | |
| | TCTCATGCAGTCACCAGCTCCCCTAACCCTATTTCTCGTT | |
| | CTCCTAACAGCGCTATCAGAGATTCGCTATCAGAAACTCT | |
| | AAATTTATTACCGGATTGGGGACCGATTGGGAGTTGGAGC | |
| | GCCCTAACCCTATTGTTAGCACTACCCTAGCCCTATTTCA | |
| | AACTCCCAACAGGCAAAATGCACCACAATTTTGACACAAA | |
| | GTCCCAATACTGGAAAATCTTGACAACCAAGGGGGCCAAA | |
| | CATGCACCATGAATTAGGCGCAAACACGGGTATTTCGACA | |
| | CAACACAGCAGAGCATACTCTAAATTACTCCCGGCCAAAC | |
| | AACACAGCACGCTCCGCAGCAAATCTATTTAAACACTCCC | |
| | ACGCCCCCCCAGAAATCCTTCTTCACACAACACAACAACT | |
| | CAACCACTCCAACAAACAAATACAATGACCCAGTCCGTCA | |
| | AGGTCGTTGAGAAGCACGTCCCCATTGTCATTGAGAAGCC | |
| | CTCCGAGAAAGAGGAAGATACCTCTTCCGAGGACTCCATC | |
| | GAGCTCACCGTCGGCAAGCAGCCCAAGCCCGTCACCGAGA | |
| | CTCGATCTCTCGACGATCTCGAGGCCATCATGAAGGCTGG | |
| | TAAGACCAAGCTGCTCGAGGACCACGAGGTTGTCAAGCTC | |
| | TCTCTCGAGGGCAAGCTGCCCCTCTACGCTCTCGAGAAGC | |
| | AGCTCGGTGACAACACCCGAGCCGTCGGTATCCGACGATC | |
| | CATCATCTCCCAGCAGTCCAACACCAAGACCCTCGAGACT | |
| | TCCAAGCTCCCCTACCTGCACTACGACTACGACCGAGTTT | |
| | TCGGTGCCTGCTGCGAGAACGTCATCGGTTACATGCCTCT | |
| | GCCCGTCGGTGTTGCCGGCCCCATGAACATTGACGGCAAG | |
| | AACTACCACATCCCCATGGCCACCACCGAGGGCTGTCTGG | |
| | TTGCTTCCACCATGCGAGGCTGCAAGGCCATCAACGCTGG | |
| | TGGTGGTGTCACCACCGTTCTCACCCAGGACGGTATGACC | |
| | CGAGGTCCTTGTGTCTCCTTCCCCTCTCTCAAGCGAGCTG | |
| | GTGCTGCCAAGATCTGGCTCGACTCCGAGGAAGGCCTCAA | |
| | GTCCATGCGAAAGGCCTTCAACTCCACTTCTCGATTTGCC | |
| | CGACTCCAGTCTCTGCACTCCACTCTCGCCGGTAACCTCC | |
| | TCTTCATCCGATTCCGAACCACCACCGGTGACGCCATGGG | |
| | TATGAACATGATCTCCAAGGGTGTCGAGCACTCTCTGGCC | |
| | GTCATGGTCAAGGAGTACGGTTTCCCCGACATGGACATTG | |
| | TCTCTGTTTCCGGCAACTACTGCACCGACAAGAAGCCCGC | |
| | TGCCATCAACTGGATCGAGGGCCGAGGCAAGTCTGTTGTT | |
| | GCTGAGGCCACCATCCCCGCCCACATTGTCAAGTCCGTTC | |
| | TCAAGTCCGAGGTCGATGCTCTGGTCGAGCTCAACATCTC | |
| | CAAGAACCTCATTGGCTCTGCCATGGCCGGCTCCGTCGGT | |
| | GGTTTCAACGCCCACGCCGCCAACCTGGTCACCGCCATCT | |
| | ACCTCGCCACCGGCCAGGACCCCGCCCAGAACGTCGAGTC | |
| | CTCCAACTGTATCACCCTCATGTCCAACGTCGACGGTAAC | |
| | CTGCTCATCTCTGTCTCCATGCCCTCCATTGAGGTCGGTA | |
| | CCATTGGTGGCGGTACCATCCTCGAGCCCCAGGGTGCCAT | |
| | GCTCGAGATGCTCGGTGTCCGAGGCCCCCACATTGAGACT | |
| | CCCGGTGCCAACGCCCAGCAGCTCGCCCGAATCATTGCCT | |
| | CCGGTGTTCTGGCTGCTGAGCTGTCTCTGTGCTCTGCTCT | |
| | GGCCGCCGGCCACCTCGTCCAGTCCCACATGACCCACAAC | |
| | CGATCCCAGGCTCCCACTCCCGCCAAGCAGTCCCAGGCCG | |
| | ATCTCCAGCGACTCCAGAACGGCTCCAACATCTGTATCCG | |
| | TAGCTAAACAGTTTGTTTAGCAAAATATATTTAACGAGTT | |
| | TGATAGAGGCGCTGGACTACATAATTACTGAATCACGCGT | |
| | ACATGTTTCAGCTCAAATTGTATCACGGTTTCTTTGTAGC | |
| | AATGGAGGGGGAGAGTTGACAAGGCATTAGAGAAGAGAGC | |
| | GAGAGGAGAAGACAAGTGGATAGACGACTGCAATCATATG | |
| | ATCTGCACAAACTGCGATGTTTTCCTGTCAGATCATGTTC | |
| | TTTTGCTCATAGTTAAGCTATCGTGACTTTACGGATCCGC | |
| | CGAGCATCTTAGTAGCGAGGTTTGTTGCCTCGTGTCTCCA | |
| | GGCCTCCTCTTGTGTGTGATTCCGAAGCATAGTGTACGAT | |
| | TGGTAAATTGGTGATGAGGGGCCTACCGTGTGGCAATCCA | |
| | GAGGCTCGAGG | |
| DNA | TATGCAAAAGCACGAAGCAAGTGTCTCCCACGTAGGCCGT | 7 |
| 53 | TGGACGCCGCTCCTGTACGCACTTTGTCCCACAAATTCCC | |
| | GATTCCGCAATTTGTTCGCCGTGCACGAAGAACTGCGGTC | |
| | AGGTGACACAACTTTTTCCATCTCAGGGTGTGTCGCGTGT | |
| | GCTTCATCCAAACTTTAGTTGGGGTTCGGGTTCGCGCGAG | |
| | ATGATCACGTGCCCTGATTTGGTGTCGTCCCCCGTCGCGC | |
| | TGCGCACGTGATTTATTTATTTCCGGTGGCTGCTGTCTAC | |
| | GCGGGGCCTTCTCTGCCCTTCTGTTTCAACCTTCGGGCGG | |
| | TTCTCGTAACCAGCAGTAGCAATCCATTTCGAAACTCAAA | |
| | GAGCTAAAAACGTTAAACCTCAGCAGTCGCTCGACGAATG | |
| | GGCTGCGGTTGGGAAGCCCACGAGGCCTATAGCCAGAGCC | |
| | TCGAGTTGACAGGAGCCCAGACGCCTTTTCCAACGGCAAC | |
| | TTTTATATAAAATGGCAATGTATTCATGCAATTGCGGCCG | |
| | TGTCAGGTTGGAGACACTGGACCACACTCTCCATTGCTTC | |
| | CTGAGGAGATGGATCATTGCTAGTGCATCTACGCGCAGCA | |
| | ATCCCGCAAGCTCGACAACCGTAGATGGGCTTTGGTGGGC | |
| | CAATCAATTACGCAACCCGCACGTTAAATTGTATGAGGAA | |
| | GGAAGGCCACGGTACAAAGTGGGTGGTCTTCACCCAGTGG | |
| | TTGTTGGTGGCGTCATGCAGACCATGCATTGGGGATAGCA | |
| | CAGGGTTGGGGTGTCTTGTGGACTCAATGGGTGAAAGGAG | |
| | ATGGAAAAGGGCGGTGAAAAGTGGTAGAATCGAAATCCCT | |
| | GACGTCAATTTATAAAGTAAAATGCGTTTCTGCCATTTTG | |
| | CTCCCCTCCTTCTTTCGCAATCGCCTCCCCAAAAGTTGTC | |
| | GTGGCAGTACACATGCTTGCATACAATGAAGCTAATCCGG | |
| | CTTGCTCAGTAGTTGCTATATCCAGGCATGGTGTGAAACC | |
| | CCTCAAAGTATATATAGGAGCGGTGAGCCCCAGTCTGGGG | |
| | TCTTTTCTCTCCATCTCAAAACTACTTTCTCACAATGTCC | |
| | AAGAAGCACATTGTCATCATTGGTGCTGGTGTCGGTGGTA | |
| | CTGCCACCGCCGCCCGACTCGCCCGAGAGGGTTTCAAGGT | |
| | CACCGTCGTTGAGAAGAACGACTTCGGTGGTGGTCGATGC | |
| | TCTCTCATCCACCACCAGGGCCACCGATTCGACCAGGGCC | |
| | CCTCTCTCTACCTCATGCCCAAGTACTTCGAGGACGCCTT | |
| | TGCCGACCTCGACGAGCGAATCCAGGACCACCTCGAGCTG | |
| | CTCCGATGTGACAACAACTACAAGGTCCACTTTGACGACG | |
| | GCGAGTCCATCCAGCTCTCTTCCGATCTGACCCGAATGAA | |
| | GGCCGAGCTCGACCGAGTCGAGGGTCCTCTCGGCTTTGGC | |
| | CGATTCCTCGACTTTATGAAGGAGACTCACATCCACTACG | |
| | AGTCCGGTACCCTCATTGCTCTCAAGAAGAACTTTGAGTC | |
| | CATCTGGGATCTCATCCGAATCAAGTACGCCCCCGAGATT | |
| | TTCCGACTCCACCTCTTCGGCAAGATCTACGACCGAGCCT | |
| | CCAAGTATTTCAAGACCAAGAAGATGCGAATGGCCTTCAC | |
| | CTTCCAGACTATGTACATGGGCATGTCCCCCTACGACGCC | |
| | CCCGCTGTCTACTCTCTGCTCCAGTACACCGAGTTTGCCG | |
| | AGGGTATCTGGTACCCCCGAGGCGGCTTCAACATGGTTGT | |
| | CCAGAAGCTCGAGGCCATTGCCAAGCAGAAGTACGATGCT | |
| | GAGTTCATCTACAACGCTCCCGTTGCCAAGATCAACACCG | |
| | ACGACGCCACCAAGCAGGTCACCGGTGTCACTCTCGAGAA | |
| | CGGCCACATCATCGACGCCGACGCCGTTGTCTGCAACGCC | |
| | GATCTGGTCTACGCCTACCACAACCTGCTGCCTCCCTGCC | |
| | GATGGACCCAGAACACCCTCGCCTCTAAGAAGCTGACCTC | |
| | CTCCTCCATCTCCTTCTACTGGTCCATGTCCACCAAGGTC | |
| | CCCCAGCTCGACGTCCACAACATCTTCCTCGCCGAGGCCT | |
| | ACCAGGAGTCTTTCGACGAGATCTTCAAGGACTTTGGTCT | |
| | GCCCTCCGAGGCTTCTTTCTACGTCAACGTTCCCTCTCGA | |
| | ATCGACCCCTCTGCTGCTCCCGATGGTAAGGACTCCGTCA | |
| | TTGTTCTCGTCCCCATCGGCCACATGAAGTCCAAGACCGG | |
| | TGATGCTTCCACCGAGAACTACCCCGCCATGGTCGACAAG | |
| | GCTCGAAAGATGGTTCTTGCTGTCATTGAGCGACGACTCG | |
| | GTATGTCCAACTTTGCCGATCTCATTGAGCACGAGCAGGT | |
| | CAACGACCCCGCCGTCTGGCAGTCCAAGTTCAACCTGTGG | |
| | CGAGGCTCCATCCTCGGTCTGTCCCACGACGTTCTCCAGG | |
| | TTCTGTGGTTCCGACCCTCTACCAAGGACTCCACCGGCCG | |
| | ATACGACAACCTCTTCTTCGTCGGTGCCTCCACCCACCCC | |
| | GGTACTGGTGTCCCCATTGTTCTGGCCGGTTCCAAGCTCA | |
| | CCTCCGACCAGGTTGTCAAGTCTTTCGGTAAGACCCCCAA | |
| | GCCCCGAAAGATCGAGATGGAGAACACCCAGGCTCCTCTG | |
| | GAGGAGCCCGACGCCGAGTCTACTTTCCCCGTCTGGTTCT | |
| | GGCTCCGAGCTGCCTTCTGGGTCATGTTCATGTTCTTCTA | |
| | CTTCTTCCCCCAGTCCAACGGCCAGACCCCCGCCTCCTTC | |
| | ATCAACAACCTCCTCCCCGAGGTCTTCCGAGTCCACAACT | |
| | CCAACGTAATTTAAAGGATATAAAAGTGCATGCACGAGAG | |
| | TGTAATAAATTTATAATCGAACGACTGACAGGCCAGTGCG | |
| | ATCCACGAGTCTGGCCGGTGACACGAGCACCATGTAGATA | |
| | ACACGACGACAAATGACGGTGACAAGAGCATGGGCGGAGT | |
| | TTGACTGATGGAGGATGTAGATCTGGATTTCCGCTGGGAT | |
| | GTCAGTATGAAATGCTGAAATTCTACCGAGTGTCCTCGTT | |
| | ATCGTGTGGTATCTGCTATATCTGCAAAGGCCATGCTGGT | |
| | GTGTTGTGGTTATCGTGTGTGGAGTGTAGATGTTCCTCGA | |
| | ACAGGATATTTCGATCTTGGATACGTACTCGCTTGTGTCT | |
| | GGTTTCGTGTGCCGGGTTGGCGGCGCATTTGTGTCCCAAA | |
| | AAACAGCCCCAATTGCCCCAATTGACCCCAAATTGACCCA | |
| | GTAGCGGGCCCAACCCCGGCGAGAGCCCCCTTCTCCCCAC | |
| | ATATCAAACCTCCCCCGGTTCCCACACTTGCCGTTAAGGG | |
| | CGTAGGGTACTGCAGTCTGGAATCTACGCTTGTTCAGACT | |
| | TTGTACTAGTTTCTTTGTCTGGCCATCCGGGTAACCCATG | |
| | CCGGACGCAAAATAGACTACTGAAAATTTTTTTGCTTTGT | |
| | GGTTGGGACTTTAGCCAAGGGTATAAAAGACCACCGTCCC | |
| | CGAATTACCTTTCCTCTTCTTTTCTCTCTCTCCTTGTCAA | |
| | CTCACACCCGAAATCGTTAAGCATTTCCTTCTGAGTATAA | |
| | GAATCATTCAAAATGCTCCTGACCTACATGGAGGTCCACC | |
| | TCTACTACACCCTGCCCGTTCTCGGTGTTCTGTCTTGGCT | |
| | CTCCCGACCTTACTACACCGCCACCGACGCTCTCAAGTTC | |
| | AAGTTCCTGACCCTCGTTGCCTTTACCACTGCCTCCGCCT | |
| | GGGACAACTACATCGTCTACCACAAGGCCTGGTCCTACTG | |
| | CCCCACCTGTGTCACCGCCGTCATTGGTTACGTTCCTCTC | |
| | GAGGAGTACATGTTCTTTATCATCATGACCCTGCTCACCG | |
| | TCGCCTTCACCAACCTCGTCATGCGATGGCACCTCCACTC | |
| | CTTCTTCATCCGACCCGAGACTCCCGTCATGCAGTCTGTT | |
| | CTCGTCCGACTTGTCCCCATCACTGCTCTGCTCATCACCG | |
| | CCTACAAGGCTTGGCACCTTGCTGTCCCCGGCAAGCCCCT | |
| | CTTCTACGGCTCTTGTATCCTGTGGTACGCTTGCCCCGTC | |
| | CTCGCTCTCCTCTGGTTTGGCGCCGGCGAGTACATGATGC | |
| | GACGACCCCTGGCCGTTCTCGTCTCCATTGCCCTGCCCAC | |
| | TCTCTTCCTGTGCTGGGTCGATGTTGTCGCCATTGGTGCC | |
| | GGTACCTGGGACATCTCTCTGGCCACTTCCACTGGTAAGT | |
| | TCGTTGTCCCCCACCTCCCCGTCGAGGAGTTCATGTTCTT | |
| | CGCCCTCATCAACACCGTCCTCGTCTTTGGTACCTGTGCC | |
| | ATCGACCGAACCATGGCCATCCTCCACCTCTTCAAGAACA | |
| | AGTCTCCTTACCAGCGACCCTACCAGCACTCCAAGTCCTT | |
| | CCTGCACCAGATTCTCGAGATGACCTGGGCCTTCTGTCTG | |
| | CCCGACCAGGTTCTGCACTCTGACACCTTCCACGACCTCT | |
| | CCGTCTCTTGGGATATCCTCCGAAAGGCCTCTAAGTCCTT | |
| | CTACACCGCTTCCGCTGTCTTCCCCGGTGACGTCCGACAG | |
| | GAGCTTGGTGTTCTCTACGCCTTCTGCCGAGCCACCGATG | |
| | ACCTCTGTGACAACGAGCAGGTCCCCGTCCAGACCCGAAA | |
| | GGAGCAGCTCATCCTCACCCACCAGTTTGTCTCCGACCTC | |
| | TTTGGCCAGAAGACCTCCGCCCCCACCGCCATTGACTGGG | |
| | ACTTCTACAACGACCAGCTCCCCGCTTCCTGCATCTCCGC | |
| | CTTCAAGTCTTTCACCCGACTCCGACACGTTCTCGAGGCT | |
| | GGTGCCATCAAGGAGCTGCTCGACGGTTACAAGTGGGATC | |
| | TCGAGCGACGATCCATCCGAGATCAAGAGGACCTCCGATA | |
| | CTACTCCGCCTGTGTTGCCTCTTCCGTCGGTGAGATGTGC | |
| | ACCCGAATCATCCTCGCCCACGCTGACAAGCCCGCCTCCC | |
| | GACAGCAGACTCAGTGGATCATCCAGCGAGCCCGAGAGAT | |
| | GGGCCTCGTTCTCCAGTACACCAACATTGCCCGAGACATT | |
| | GTCACTGACTCCGAGGAGCTCGGCCGATGCTACCTCCCCC | |
| | AGGACTGGCTCACCGAGAAAGAGGTTGCTCTCATCCAGGG | |
| | CGGTCTTGCTCGAGAGATTGGTGAGGAGCGACTCCTCTCT | |
| | CTGTCCCACCGACTCATCTACCAGGCTGACGAGCTCATGG | |
| | TCGTCGCCAACAAGGGTATCGACAAGCTCCCCTCCCACTG | |
| | CCAGGGCGGCGTCCGAGCTGCCTGCAACGTCTACGCCTCC | |
| | ATCGGTACCAAGCTCAAGTCCTACAAGCACCACTACCCCT | |
| | CTCGAGCCCACGTCGGTAACTCCAAGCGAGTCGAGATTGC | |
| | TCTGCTGTCCGTCTACAACCTGTACACCGCCCCCATTGCC | |
| | ACCTCCTCCACCACCCACTGCCGACAGGGCAAGATGCGAA | |
| | ACCTCAACACGATCTAAACTATTTACAGCATGTGTAATGA | |
| | GGAATATAACGTTGATTGAATTGTTTGTGAAAAATGTAGA | |
| | AAATTTCAGTGAAGTTGTGTTTTCTATATAGTAAGCACTT | |
| | TTGGTACAAGTATCTGCACATCCCTGCATGTTACAAGCCT | |
| | GATCATGCAGGGCAATATTCTGACTATAAATATACCTCGA | |
| | TATTTTAGCAAGCTATAGTTGCACTAACTAACCAACTAGT | |
| | ATCTTCTAAACCTCTCTGCACCTTCTTTCGCTTGCACTGT | |
| | TCCCAGACTCCAAGATTTGTATACGTGCTTGACAAATAGC | |
| | TCAAGGGCGTGGAGCACAGGGTTTAGGAGGTTTTAATGGG | |
| | CGAGAAGGCGCGTAGATGTAGTCTTCCTCGGTCCCATCGG | |
| | TAATCACGTGTGTGCCGATTTGCAAGACGAAAAGCCACGA | |
| | GAATAAACCGGGAGAGGGGATGGAAGTCCCCGAACAGCAA | |
| | CCAGCCCTTGCCCTCGTGGACATAACCTTTCACTTGCCAG | |
| | AACTCTAAGCGTCACCACGGTATACAAGCGCACGTAGAAG | |
| | ATTGTGGAAGTCGTGTTGGAGACTGTTGATTTGGGCGGTG | |
| | GAGGGGGGTATTTGAGAGCAAGTTTGAGATTTGTGCCATT | |
| | GAGGGGGAGGTTATTGTGGCCATGCAGTCGGATTTGCCGT | |
| | CACGGGACCGCAACATGCTTTTCATTGCAGTCCTTCAACT | |
| | ATCCATCTCACCTCCCCCAATGGCTTTTAACTTTCGAATG | |
| | ACGAAAGCACCCCCCTTTGTACAGATGACTATTTGGGACC | |
| | AATCCAATAGCGCAATTGGGTTTGCATCATGTATAAAAGG | |
| | AGCAATCCCCCACTAGTTATAAAGTCACAAGTATCTCAGT | |
| | ATACCCGTCTAACCACACATTTATCACAATGACCGATGTC | |
| | CGATTCCGAATCATCGGTACCGGTGCCTACGTTCCCGAGC | |
| | GAATTGTCTCCAACGACGAGGTTGGTGCTCCCGCTGGTGT | |
| | TGACGACGACTGGATCACCCGAAAGACCGGTATCCGACAG | |
| | CGACGATGGGCTGCTGACGACCAGGCCACCTCCGATCTCG | |
| | CCACCGCTGCCGGCCGAGCTGCTCTCAAGGCTGCTGGTAT | |
| | CACCCCCGAGCAGCTGACCGTCATTGCCGTTGCCACCTCT | |
| | ACCCCCGACCGACCCCAGCCTCCCACTGCCGCCTACGTCC | |
| | AGCACCACCTCGGTGCCACCGGCACCGCCGCCTTCGATGT | |
| | CAACGCCGTCTGCTCCGGTACCGTCTTTGCCCTCTCTTCC | |
| | GTCGCCGGTACTCTGGTCTACCGAGGCGGCTACGCCCTCG | |
| | TCATTGGTGCTGACCTCTACTCCCGAATCCTCAACCCCGC | |
| | CGACCGAAAGACTGTTGTTCTCTTCGGTGACGGTGCCGGC | |
| | GCCATGGTTCTCGGCCCCACCTCCACTGGTACCGGCCCCA | |
| | TTGTCCGACGAGTTGCTCTGCACACCTTCGGTGGTCTGAC | |
| | CGATCTCATCCGAGTCCCCGCCGGTGGCTCTCGACAGCCT | |
| | CTCGACACCGACGGTCTGGACGCCGGTCTGCAGTACTTCG | |
| | CCATGGACGGCCGAGAGGTCCGACGATTCGTCACCGAGCA | |
| | CCTGCCCCAGCTCATCAAGGGCTTCCTCCACGAGGCTGGT | |
| | GTCGACGCCGCTGACATCTCCCACTTTGTCCCCCACCAGG | |
| | CCAACGGTGTCATGCTCGACGAGGTCTTTGGCGAGCTCCA | |
| | CCTCCCCCGAGCCACCATGCACCGAACCGTCGAGACTTAC | |
| | GGCAACACCGGTGCTGCCTCCATCCCCATCACCATGGACG | |
| | CCGCCGTCCGAGCCGGTTCCTTCCGACCCGGAGAGCTCGT | |
| | TCTGCTCGCCGGCTTCGGCGGTGGTATGGCCGCCTCTTTC | |
| | GCCCTCATTGAGTGGTAAACCAACAGTAGCATGACGAGTA | |
| | TATAGTGTTTTATGAATTTAATGTGTATACAAGGGTGTGT | |
| | GTAGTTGAGTAATGAGAAACCCCAATCATCATCTGCCTAA | |
| | AGCTAACGAGGATTGTAACGACACCGTATGTACTCACCAC | |
| | AGGTACAAAGTACAACTACGAGCATGTACAGTATACCAAG | |
| | ATCGGTGATGACTTGAATTTGTACAGCTCAAACCCCCCAG | |
| | ATACGGTATAATACACAGTTCTGTACAAGCCGTCACAATT | |
| | GCCAAAGTTGCAAAAATGATTGTATAAAAATATATGCTCC | |
| | TCCCAATACAAGGCAGAACTTTCCTCCACCGTATCCGCTC | |
| | GCAATTTCTTTCGTGCGGGAAGCGAACCGGCAATATTCCA | |
| | CAATGTGCTGGCATTTACTTGTGCTGGCAAAAGAGGCACA | |
| | AAGAATACTTGTAGTCGGAGCCACTCACTGTCCCACAAAT | |
| | AGCTCCCCGCTGTCAATCTCTCCTGCACCGCCTGCTCACA | |
| | TGGATGCTAAGCCGCACTAGGTCGCATATATGGCTCTGCA | |
| | CTAAAAATTAGGGGTCAACCACAGTGCGGTATTTTTAGAT | |
| | TCGCACCAAGCAGCGAGTAAGCAAAAATACGCCTACCGGG | |
| | GTCCGATATTATTCAGGAGGTGCCATTAGAGGAGGGCAGA | |
| | TGAGAGTCGGATATCGGAGATATTACCGAGGCTATAATTA | |
| | CCCCATCCACGCCTTTCACCCCTCCCACTCTCTCCCTCAC | |
| | CGCACACCAACCCACCACTTTCAAAATATACCGCAACATT | |
| | GACATAATCTCCGGTACAGTGGTTAGCACCGAGAGGACCC | |
| | CAAAAAGCTTGGGGGAGATAGAGGTAGGCTTTTTTTTGTC | |
| | AGTCAAATCGTATATGCCAATACACACACACACACACACA | |
| | CACACACACAGTTTCGTACATAACAGTATATTGGAAGGGA | |
| | GTGTGCTTGGCAAAGACAGGAGAAGACGGTGCTGTTAGAG | |
| | GGCAATCCAGACGGGCTAGAGCGCTGTAACTTTCGGATCG | |
| | ATTTCAATTCCTCTAGAATACCAAATACCAGTGGTTAAGC | |
| | GGCTCATTTACCAGTCCTAATACCCCCTCCACCAGCCACC | |
| | TTCCCCTATTCCTCGGCAGTGCTTTTTTACCTTTGAGATG | |
| | TGGCCTTGTCTCCGTTACTTCCCAACCGTGAGTGCTGTGT | |
| | GGTGTGCTGGACAGTGCGACATAACTAACCCTAACCCAGA | |
| | CGAGCCAGCGCACCCCAATTTTGTGTTTGCCAACTCCTAC | |
| | TTTTCTCCTCTCCTCCATCGGTATTTCATCGACAAATCTC | |
| | TTTGCTACCAACAACCACACAAATTAAAAATGGACTACAT | |
| | CATCTCCGCTCCCGGCAAGGTCATCCTCTTCGGTGAGCAC | |
| | GCCGCCGTCTTTGGCAAGCCCGCCATTGCTGCTGCCATCG | |
| | ATCTGCGAACCTACCTCCTCGTCGAAACCACCACCTCCGA | |
| | CACCCCCACCGTCACTCTCGAGTTCCCCGATATCCACCTT | |
| | AACTTCAAGGTTCAGGTCGACAAGCTCGCCTCTCTCACTG | |
| | CCCAGACCAAGGCCGACCACCTCAACTGGTCCACCCCCAA | |
| | GACCCTCGACAAGCACATCTTCGACTCTCTCTCTTCTCTC | |
| | GCTCTGCTGGAGGAGCCCGGTCTGACCAAGGTCCAGCAGG | |
| | CTGCTGTTGTTTCTTTCCTGTACCTCTACATCCACCTCTG | |
| | TCCTCCCTCCGTCTGCGAGGACTCCTCCAACTGGGTTGTC | |
| | CGATCCACCCTCCCCATTGGTGCTGGCCTCGGTTCTTCTG | |
| | CCTCCATCTGTGTCTGCCTCGCCGCCGGCCTCCTCGTTCT | |
| | CAACGGCCAGCTCTCCATCGACCAGGCTCGAGACTTCAAG | |
| | TCTCTGACCGAGAAGCAGCTGTCTCTGGTCGACGACTGGT | |
| | CCTTCGTCGGTGAGATGTGCATCCACGGCAACCCCTCCGG | |
| | TATCGACAACGCCGTTGCCACCCAGGGTGGTGCTCTGCTG | |
| | TTCCAGCGACCCAACAACCGAGTTCCTCTCGTTGATATCC | |
| | CCGAGATGAAGCTGCTGCTCACCAACACCAAGCACCCCCG | |
| | ATCCACTGCCGATCTGGTCGGTGGTGTCGGTGTTCTCACC | |
| | AAGGAGTTTGGCTCCATCATGGACCCCATCATGACCTCCG | |
| | TCGGCGAGATCTCCAACCAGGCCATGGAGATCATCTCCCG | |
| | AGGCAAGAAGATGGTTGACCAGTCCAACCTCGAGATTGAG | |
| | CAGGGTATCCTCCCCCAGCCCACTTCCGAGGACGCCTGCA | |
| | ACGTCATGGAGGACGGTGCCACCCTCCAGAAGCTCCGAGA | |
| | CATTGGCTCCGAGATGCAGCACCTCGTCCGAATCAACCAC | |
| | GGTCTGCTCATTGCCATGGGTGTCTCCCACCCCAAGCTCG | |
| | AGATCATCCGAACCGCCTCCATTGTCCACAACCTCGGTGA | |
| | AACCAAGCTCACCGGTGCTGGTGGTGGTGGCTGTGCCATC | |
| | ACCCTCGTCACCTCCAAGGACAAGACCGCCACCCAGCTCG | |
| | AGGAGAACGTCATTGCCTTCACTGAGGAGATGGCCACCCA | |
| | CGGCTTCGAGGTCCACGAGACTACCATTGGCGCCCGAGGT | |
| | GTCGGTATGTGCATTGACCACCCCTCTCTCAAGACTGTCG | |
| | AGGCCTTCAAGAAGGTCGAGCGAGCCGATCTCAAGAACAT | |
| | TGGCCCCTGGACCCACTAAACGTAAAAGCGGTATGTATTA | |
| | ATTGAGAGTTTTATAAGACAGTGTTTGCGCGAGCTGACGA | |
| | GTGTTTGCCGAGTGCATATCACAACGAGAACCTGAGAGCC | |
| | ACGCAATGTGTGGTTGTAGTGTGGATATGCCCATGAGACA | |
| | ATGGTAGTGTGGAGAATACCCATGAGACGATGGTAATGTA | |
| | AAGAATGGCCCACGAGATGCAATTGAGGCCCAATTGACTA | |
| | ATTGATATTGTAGACCGAGTCAATGGGTCAGTGCAGAGGA | |
| | GCGGCGTTTGCTCAATTGACTCGGGAACGGACATGTGCAC | |
| | CTCGGTGTTGCACACGAATACGACACTTCCCAATACACTT | |
| | TGCCTCTCCAAGGGGTAACATGATGTAATGGCGCGTTCCA | |
| | CAAGACCCAAGACATTTGTTACT | |

1. A composition of matter for use in a food, feed, pharmaceutical, or beauty care product comprising, consisting of, or consisting essentially of
(1) a retinoid component comprising a mixture of cis-isomers and trans-isomers; and
(2) a fermentative residue thereof;
wherein, the retinoid component is present, relative to the fermentative residue thereof, in a ratio by weight of greater than 4:1;
wherein the cis-isomers are present by weight, relative to the weight of the entire retinoid component, in an amount of less than 3 wt. %.

2. The composition of claim 1, wherein the fermentative residue comprises fatty acid retinyl ester (FARE), retinal, retinol, farnesol, a fermentation carbon source, or β-carotene.

3. The composition of claim 2, wherein composition possesses a weighted select fermentative residue value (WSFRV) between 0.1 and 2.5, wherein WSFRV is determined according to the following formula:
WSFRV=C+0.1×F
wherein
C=the percentage by weight of cis-isomers present relative to the total amount of retinoid component (i) and fermentative residue (ii), and
F=the percentage by weight of FARE present relative to the total amount of retinoid component (i) and fermentative residue (ii).

4. The composition of claim 1, wherein the retinoid component comprises, consists of, or consists essentially of compounds according to formula (I): 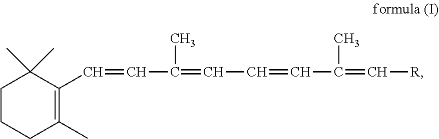
R is —CHO, —CH₂OH, —COOH, —CH(R¹)₂, —CH₂OR², —COOR³, —CONHR⁴, or —CO(NR⁴)₂,
R1 are independently lower alkoxy or R¹′ and R¹″ taken together are lower alkylenedioxy,
R² is alkanoyl or aroyl,
R³ is alkyl, aryl or aralkyl; and
R⁴, R^{4′} and R^{4″} are independently hydrogen, alkyl, aryl or aralkyl;
wherein the term “lower alkoxy” is an alkoxy group with 1 to 6 carbon atoms, such as, for example, methoxy, ethoxy or propoxy;
the term “lower alkylenedioxy” is such a group which likewise contains 1 to 6 carbon atoms, e.g., methylenedioxy or ethylenedioxy and wherein the alkyl or alkylene part can be straight-chain or branched depending on the number of carbon atoms;
the term “alkanoyl” refers to either straight-chain or branched alkanoyl groups with 1 to 18 carbon atoms, such as, for example, formyl, acetyl, propionyl, butyryl, stearoyl and palmitoyl;
the term “aroyl” refers to aromatic carboxylic acids with 7 and 11 carbon atoms, including benzoyl or naphthoyl, respectively;
the term “alkyl” is a straight-chain or branched alkyl group with 1 to 18 carbon atoms, e.g., methyl, ethyl, propyl, butyl, decyl, dodecyl, hexadecyl or octadecyl;
the term “aryl” as such or as part of “aralkyl” is phenyl or naphthyl; and
the term “aralkyl” embraces such groups with 1 to 4 carbon atoms in the aliphatic part, e.g., benzyl and phenylpropyl.

5. The composition of claim 4, wherein the retinoid component comprises, consists of, or consists essentially of retinol or retinyl esters, wherein the retinyl esters comprise, consist of, or consist essentially of retinyl acetate or retinyl palmitate.

6. (canceled)

7. The composition of claim 2, wherein the retinoid component comprises a mixture of cis- and trans-isomers, wherein the cis-isomers are present by weight, relative to the weight of the entire mixture, between 0.1-3 wt. %.

8. (canceled)

9. The composition of claim 2, wherein the fermentative residue comprises FARE, beta-carotene, retinol, retinal, and a fermentation carbon source.

10. The composition of claim 9, wherein the fermentative residue comprises retinol and retinal, wherein retinol comprises E-retinol; and wherein the retinal comprises 9Z-retinal, wherein 9Z-retinal is present, relative to the total weight of the retinoid component, in an amount between 0.1 to 10 wt. %.

11. (canceled)

12. The composition of claim 2, wherein the fermentative residue comprises one or more FAREs, wherein, relative to the total weight of retinoid component present, the one or more FAREs are present in an amount of between 0.1 to 4 wt. %.

13. (canceled)

14. The composition of claim 12, wherein the FARE comprises retinyl palmitate and/or retinyl oleate.

15.-16. (canceled)

17. The composition of claim 12, wherein the fermentative residue comprises β-carotenes, wherein, relative to the total weight of retinoid component present, the β-carotenes are present in an amount of less than 4 wt. %.

18. (canceled)

19. The composition of claim 12, wherein the fermentative residue comprises farnesol, wherein, relative to the total weight of retinoid component present, farnesol is present in an amount of between 2-10,000 parts per million.

20. (canceled)

21. The composition of claim 12, wherein the composition is free-from farnesol.

22. The composition of claim 2, wherein the fermentation carbon source comprises a biogenic carbon source.

23. The composition of claim 22, wherein the fermentation carbon source comprises linear alkanes, free fatty acids, ethanol, glucose, triglycerides, and vegetable oil, including the respective free fatty acids therefrom.

24. The composition of claim 2, wherein the fermentative residue comprises ethanol or glucose, wherein, relative to the total weight of retinoid component present, ethanol or glucose is present in an amount of between 0.05 to 2 wt. %.

25. (canceled)

26. The composition of claim 1, wherein the fermentative residue comprises an isoparaffinic fluid in an amount, relative to the weight of the entire retinoid component (i) and fermentative residue thereof (ii), of less than 1.25 wt. %.

27. (canceled)

28. The composition of claim 26, wherein the isoparaffinic fluid comprises Isopar M.

29. The composition of claim 2, wherein the fermentative residue comprises a dihydro-form of the retinoid component.

30. The composition of claim 29, wherein the dihydro-form is present, relative to the weight of the entire the retinoid component (i) and the fermentative residue thereof (ii) in an amount of less than 0.5 wt. %.

31. (canceled)

32. The composition of claim 30, wherein the dihydro-form of the retinoid component comprises dihydro-retinyl acetate.

33. The composition of claim 2, wherein the fermentative residue comprises fewer than 0.5 wt. % of rosafluene, phytoene, ergosterol, and dihydro-beta-ionone.

34. The composition of claim 2, wherein the cis-isomers are present by weight, relative to the weight of the entire mixture of cis- and trans-isomers, in an amount of less than 1 wt. %;
and/or
wherein, relative to the total weight of the retinoid component, FARE is present in an amount of less than 1.5 wt. %.

35. (canceled)

36. The composition of claim 2, wherein the retinoid component is present, relative to the fermentative residue thereof, in a ratio by weight between 4:1 to 100:1.

37. The composition of claim 2, wherein the retinoid component comprises, consists of, or consists essentially of retinyl acetate, and wherein the retinyl acetate is present, relative to the fermentative residue thereof, in a ratio by weight of greater than 25:1.

38.-39. (canceled)

40. The composition of claim 2, wherein a bio-based carbon content of the retinoid component (i) and the fermentative residue thereof (ii) is greater than 90%, wherein the bio-based carbon content is determined by ASTM D6866-20 or ¹⁴C and ¹³C isotope characterization.

41. (canceled)

42. The composition of claim 37, wherein the amount, measured relative to the weight of the entire composition, of retinyl acetate is within 5 wt. % of the amount of the retinyl acetate present in the composition after it is subjected to a heat stability test whereby the composition is heated to 105 degrees Celsius for 3 hours.

43. The composition of claim 2, wherein the composition is present in a crystalline form.

44. The composition of claim 43, wherein the crystalline form comprises a plurality of crystals having an average particle length D₅₀ as determined by a microscopic imaging method, of between 200-800 micrometers.

45.-46. (canceled)

47. The composition of claim 1, wherein the retinoid component (i) and the fermentative residue thereof (ii) are present as an emulsion in oil, wherein the emulsion in oil comprises a fat-soluble antioxidant.

48.-202. (canceled)


SEQUENCE LISTING (7 sequences)

SEQ ID NO: 1 - DNA, 8089 bp, synthetic construct
  source 1..8089: mol_type = other DNA
aagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatgatggctccctccattcgaaagttctttgctggtggtgtgtgtcgaactaacgttcagcttcccggtaaggtggttgtcatcactggtgccaacaccggcattggcaaggagactgcccgagagctcgcttcccgaggagcccgagtttacattgcttgccgagatgttctgaagggcgagtctgctgcctctgagattcgagttgacactaagaactcccaggtgctcgtgcgaaagctcgacctttccgacactaagtctatccgagcctttgctgagggctttctcgctgaggagaagcagcttcacattctgattaacaacgctggagttatgatgtgtccttactctaagactgctgatggtttcgagactcacctcggagtcaaccacctgggccacttcctcctcacctacctgctcctggagcgactcaaggtgtctgcccctgcccgagtggttaacgtttcctccgtggctcaccacattggcaagattcccttccacgacctccagtccgagaagcgatactcccgaggttttgcttactgccactccaagctggccaacgttctctttacccgagagctggccaagcgactccagggaaccggcgtcaccacctacgccgttcaccccggtgtcgtccgatccgagctggtccgacactcctccctgctctgcctgctctggcgactcttctcccccttcgttaagaccgcccgagagggtgcccagacctccctgcactgcgccctggctgagggcctggagcccctgtctggcaagtacttctctgactgcaagcgaacctgggtgtctccccgagctcgaaacaacaagactgccgagcgactctggaacgtttcctgtgagcttctcggtattcgatgggagtagacgcgtgcaattaacagatagtttgccggtgataattctcttaacctcccacactcctttgacataacgatttatgtaacgaaactgaaatttgaccagatattgttgtaaatagaaaatctggcttgtaggtggcaaactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcatctagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgagg

SEQ ID NO: 2 - DNA, 8159 bp, synthetic construct
  source 1..8159: mol_type = other DNA
aagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagcccacaaaaatgacccgatccaagttcaccgagtacaagcaggttctccgaaactcttgtgttggcaagccccagtacactgagaaggattaccccgatctcgacggaaagaactttctcgtcactggtgctactggtggagttggtctggaggccactaagctgctccttgagaagaagtctcacgttattatggttggccgatctaagactaagtctcagtctacccttgacgagcttcagaagacttactctcacggtactttcgatttcgttgaggctgatctctctgaccttaccactgttgagcgagccggagagtacattcgatctaagtacactacccttgacggtgccattcttaacgctggagtcatggctcccccctactctctcactccccagggtcacgagtctcagtggggtattaacgttgttgctcactttctcctgtctaagtacatctctcctgctctgatttctgctgctcagactgcccccaaggacaccgttcgacttgtttgggtgtcttcttctgtcgttgccatgtctccttacgagggaggtattaagtttgacgatattaaccactctaaggtcaagaacccttctccttggaccctttactctcagtctaagattggtgatgcttacctggcttacctttggtctaagcaccaccccgattctggagtcctttctgtctctctggaccccggtaacctggcttctgatctctctcgacacacttcttggctttcttctattaagaactacgttctttaccctcccaagtacggtgcctacactgagctgtctgctcttctcaacccttccgttaagaacaacgagcaccttattccctggggtattgagggacaccttcgacaggatgtcgacgatggtcgacgaggaaaggacggagaggagctctggcagggtcttaacaaggatgtcgagggtttctttaaggaggagtaaacgcgtgcaattaacagatagtttgccggtgataattctcttaacctcccacactcctttgacataacgatttatgtaacgaaactgaaatttgaccagatattgttgtaaatagaaaatctggcttgtaggtggcaaactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcatctagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgagg

SEQ ID NO: 3 - DNA, 14006 bp, synthetic construct
  source 1..14006: mol_type = other DNA
gcggagcctaggccggccagcgtggcgcgtggccatattggccagcagcttactacagctcagccaaagtggatcatgatggacggaaatatcggactggaggccaaaaaggaggtgctcaaatacgcacgggacaagtctgcacaggtggcattcgaacccacgtctgtccccaaagccgctgctctttccgagctcaatctgcccgtttaccccaacaactccattgccttagccacccccaacactgcagagctcgaggccatgttcgaggcattccacgagaagggccgatttgacgttgacgactggtttccagtcatagacggtcttgctctcggagctgactttagaaacggagccaccatgttgtcgcaccaacatcgtggactcaaagctattctcgaacaaggaactctggcccaggcaatccacatgctaccatacatcccaactttgatcatcaagggcggagccaacggcgtcgtcgtcttccaactcattgatgatatcgagtctgccattcattcacagcgttctgcttccaataaaacgcctggcctgttccagaagggcaacgctgcgagtggaaacaccaaggtcggcgtctacatgcagtactttgagcccgaagaagtgggcagtcagtcgattgtaagcgtgactggagctggagacactctgtttggaaccctggccatggagattgtcaaggacgagtcctggttgaacgatatgggagacaagaagagtgcagttgtttctcgagccatgaacaatgctgtgaagactattcagagtaaggacgctgtctgcaagagcatcctttaagtgatttgccatgctttctcttcttccacgatgtaaatacttattttacacactactgtgcagtagcaaatacagaacaagagttgtcgcctattgacagtacagtacgagtagtgtatgtacagtagttatacaatatctatgtgaaattcgtcggcagctttcggctgatgaactacgagttcttcgtaataaatcatcaacgtaataagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatgggtgatctcgatgcccgaggaacctctgctcaccccgagctctctgagcgaccttctattatgccttctatgtctgatattcaggacccttctggtgatgacaaggctactccccgaggatctgctgctggtctgccccagcttgagcttgctggacacgcccgacgacttggccaccttgagaacttctttgctgtccaggctcgacagcagatttactcttcttttgctgttttttgtgagtttgacactgcttgttctctcgctcagcttgcttctgctgtgcgaaacgtttgtctttctaacccccttctccttcacactgttgagcctaagcaccctgacatcgctggattctaccactctgacgagtacctttcccgaccttggcctcagcacgattacatgcgagttcttcgagaggttcacgtcgctgacgttgttatgaacggacagaaggagcacgctcacgttgttcgagatgctgttgacgtttttcaggctcacggaaaccaggttacttctgagctccttgagcttatgactcagattgagattcctcacgcttctcagactcgaccctcttggcgacttctctgttttccccacggagaggctaaccgatggcgaacctttgcttttgtttctaaccactgttcttctgatggtctttctggtcttaacttctttcgagatctccagaaggagcttgctcacggccccacctctggtgctcctggtgcccccggagcttccggagttattttcgattacgctcaggacgctgctaccctgcccaagctgccccctcccattgatcagaagctcgattaccgaccttctaagaaggctcttctcggccttctcgctggcaagttcgttcgagagaagctcggttacgtttctgctgctcctcccactacccctacctctgaccttgctcaccctgagggtcaccagtactactgttaccttgttaacgttcccacttcttctgttgcccacattaagactcaggtgcgagagaacgttcctcacaagtgtactctcactccctttctccaggcttgttggcttgtttctctgttcaagtacggtcgagttttttctggttcttggcttgagcgatacaccgatgttcttgttgctatgaacactcgacagcttctccccgaggaccttgagcttcagcgacagtaccgatacggttctaacattggagctgttcgatacaactaccctattgctccccttgacgttcgagataacgatcagaagttctggtcccttgttgagtcttaccgacttgccctttctgatgcccgagataagaacgattacctttacgctcttggtgctcttatgctccctgagatttacgagaagaagaacgttgatgctgttgttaacgataccattcttaaccagcgacgacagggaacccttctttctaacgttggttacgttcgagatgagcagcccactgcttttgctattaagaaccacgttttttctcagggagttggagctaaccgaaacgcttttgttcttaacatttgtgctaccgatcagggtggtcttaacatcgctatttctattgctaagggaacccttgcttctcgacaggagggacaggagctttgtgatatttttaagtctactctccttcgattttaaacgcgtctatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatctgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtaccattccaaccgtctcagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcgtagaccccggtgtatataaaggggtggagaggacggattattagcaccaacacacacacttatactacatgctagccacaaaaatgctctctttcttctggcgaaacggtatcgagactcccgagcccctcaaggctgacgtttccggctctatccctccctggcttcagggaacccttctccgaaacggtcctggtctgttctccgttggcaacacttcctacaagcactggttcgatggtatggctctcattcactccttcacctttaaggatggtgaggttttttaccgatctaagtacctgaagtctgagacttacaagaagaacatcgctgccgaccgaatcgttgtgtctgagttcggaaccatggtgtaccccgatccctgcaagaacattttctcccgagccttctcttacatgatgaacgccattcctgactttaccgataacaacctcattaacatcattaagtacggtgaggattactacgcctcctctgaggtcaactacatcaaccagattgaccccctgacccttgagactctcggacgaactaactaccgaaaccacattgccatcaaccttgccactgctcaccctcactacgacgaggagggtaacacttacaacatgggcactgctattatgaacctcggtcgacccaagtacgtgattttcaaggtgcccgccaacacctctgataaggagaacaagaagcctgccctctctgaggtggagcaggtttgctccattcccatccgaccctccctttacccttcttacttccactcttttggcatgactgagaactacatcatcttcgttgagcaggccttcaagctggacatcgtcaagctggctactgcttacttccgagatattaactggggatcttgccttaagttcgaccaggatgacattaacgtgttccacctggtcaacaagaagactggtaaggctgtgtccgtgaagtactacactgacccctttgttaccttccaccacatcaacgcttacgaggacgatggccacgtcgtcttcgatctcattacttacaaggactctaagctgtacgatatgttctacattcagaacatgaagcaggacgtcaagcgatttattgagactaacaaggacttcgctcagcccgtgtgccagcgatttgtccttcccgtcaacgttgataaggagactcctcaggacatcaaccttgtcaagctgcaggacaccactgccactgctgtcctgaaggaggacggctctgtctactgcacccctgacatcatttttaagggtcttgagctccctgctatcaactacaagtttaactctaagaagaaccgatacttctacggcacccgagtggagtggtccccttaccctaacaaggtcgctaaggtggacgttgttactcgaacccacaagatttggactgaggaggagtgttacccttctgagcctgtctttattgcctcccctgacgccgttgatgaggatgacggtgtgattctttcttctgtggtttctttcaacccccagcgaccccctttcctggttgtcctcgatgctaagtccttcaaggagattgctcgagctaccatcgatgcctctattcacatggaccttcacggccttttcatccacgacaagtctacctaagttttttgatcaatgatccaatggctttcacatacccccccacgcctataattaaaacacagagaaatataatctaacttaataaatattacggagaatctttcgagtgttcagcagaaatatagccattgtaacaaaagccggctatcgaccgctttatcgaagaatatttcccgccccccagtggccaaacgatatcggtcagaaggggcagctctaaacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacatgctagccacaaaaatgaccactaagtacacttccgttcacgagtctcccaacggccctggtgacgctcgacccaccgcttcccagattatcgacgattacaaccttgagggagagctttctggcaagactgttctcgtcaccggctgttcctctggtattggtgttgagactgcccgagctatttaccgaactggtgccaccctttacctcactgcccgagatgtcgataaggccaagaccgttcttcccgaccttgttgacacttcccgagtccactttctccaccttgaccttaactctctggagtctgttcgaggttttgctgagaacttcaagtctaagtccactcagcttcacattctcatcgagaacgctggcgtgatggcctgtcccgagggccgaaccgtcgatggttttgagactcagtttggtatcaaccaccttgctcactttctcctcttttacctcctcaaggatacccttctcaactcttctacccccgctttcaactcccgagttgtcatcctctcttcttgtgctcaccaggctggttccgttcaccttaacaacctgtctcttgagggtggatacgagccttggaagtcttacggccagtccaagactgccaacctttggactgcccgagagatcgagaagcgatttggtgcttccggtatccactcttgggctgttcaccccggttccatcgctactgagcttcagcgacacgtttccgacgagcttaagcagaagtgggctgacgataaggagggtgccaagctgtggaagtccaccgagcagggtgccgccaccactgtccttgctgctgtttcccctgagcttgagggtaagggcggtctttaccttgaggatacccaggttgccaagccccctgcccgaggaatgtttggtgttgctgactgggcttacgatgaggatggcccctctaagctctgggccaagtctcttgagctccttaagctccagtaaaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcacactattatcacattactacatccaaacccctacagggggaggagctctccaatcaaatatgtacattaactatctctcgtaaatcattgttataagaccgtccgtgactgtctaaatcggttcattcgttgtaacaaatcagtgtaacaactcgtggtacgcccggtttgcttcgctcacgctcccctagattttccgtctaggacacaacaagccctggtggatgacgttcaacgccttcagcgcgtctttttcctcaataacacacgagatgttaatttcgttggctccctgagaaatcatctcaatgttaatacctgcctgggccagagtagagaagaacatgcctgcacatccgaccatggccttcattctcgttccgatcagcgacacaatggtcatgcctcgcttcacatctactgtgccgtactttcgcagctcctccacagcctgcttgaggttggagtcgggagcatggaaggccatcgacacatggacctcagaagtggagatgagatcgacgacaagtttctgctggtcgagagtagcaaagatcttgttcaagaagccatgactcttggttcgcttattcgaatgaacgttgagaacggtgatattcgatttggtagtgacagctgtaggcttcttctcgtcatctacaggagcagacacctggggagtgaatgagccagaggacagagaagttagcgagtcggtggaagaagccagatcctggacattgtttttcgttttcaggttgcccgagccgtcagcagatgggtagatgatagttcctcctcccagggggttttccacgtttttgattcggataggaatatgggccttaatgacctgctccatggtgaaggggtggatgacttcagatccgtagtaggtcaactcagcggcctcctcgggggtaatgatgggcagcagacgggcagtggacacctttctgggatcggcagtgaagacaccatcgacttctttccagatctgaagctccttggcgtccagacccacagccaccagagcagcacacagatcggtgtatcctcgtccgatctggcttaggaggcctcccttgacggggccaaagaaaccggtcaacacgggcaccatgtttcccttgacaggagcagagtccgtgttaggagagccgggaagagtgatgatctcgcccagaacacgtccgaggtccgaatagaacttgggatcttcggcattggtggtggtaactgcgtgagacaggtcaaagtaccgtgcgttaacaccagcgtcccgcataactgcagtcatgtacatgcaactgagcttctccccaatggccatgatggagtcgagtgttcggggggagatctcagaaatgatttcagcagcagccaggattcgcaacagctggtcgcactcgccgttgatgtcggcattgaggttttcgagcagttcggggttcttgacgtcacgcttagcagctgcaagatggtcttctcggatagactcaatgatggggttgtaggcgtctgatccgagaagagcagagtcagcagcagcaataagacgggtggtggttccctcggccttggtggcctctagatggcctccttggccccattccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgag

SEQ ID NO: 4 - DNA, 16291 bp, synthetic construct
  source 1..16291: mol_type = other DNA
gtctggccacctaggccggcctcgcggtcagaaggggcagctctaaacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacatgctagccacaaaaatggattataacagcgcggatttcaaggagatctggggcaaggccgccgacaccgcgctgctgggaccgtacaactacctcgccaacaaccggggccacaacatcagagaacacttgatcgcagcgttcggagcggttatcaaggtggacaagagcgatctcgaaaccatttcgcacatcaccaagattttgcataactcgtcgctgcttgttgatgacgtggaagacaactcgatgctccgacgaggcctgccggcagcccattgtctgtttggagtcccccaaaccatcaactccgccaactacatgtactttgtggctctgcaggaggtgctcaagctcaagtcttatgatgccgtctccattttcaccgaggaaatgatcaacttgcatagaggtcagggtatggatctctactggagagaaacactcacttgcccctcggaagacgagtatctggagatggtggtgcacaagaccggaggactgtttcggctggctctgagacttatgctgtcggtggcatcgaaacaggaggaccatgaaaagatcaactttgatctcacacaccttaccgacacactgggagtcatttaccagattctggatgattacctcaacctgcagtccacggaattgaccgagaacaagggattctgcgaagatatcagcgaaggaaagttttcgtttccgctgattcacagcatccggaccaacccggataaccacgagattctcaacattctcaaacagcgaacaagcgacgcttcactcaaaaagtacgccgtggactacatgagaacagaaaccaagagtttcgactactgcctcaagagaatccaggccatgtcactcaaggcaagttcgtacattgatgatctcgcagcagccggccacgatgtctccaagttgcgagccattttgcattattttgtgtccacctctgactgtgaggagagaaagtactttgaggatgcgcagtgaacgcgtctatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtattagtgcaatcacatgttgctactgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtaccattccaaccgtctcagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcggagaccccggtgtatataaaggggtggagaggacggattattagcaccaacacacacacttatactacatgctagccacaaaaatgtccaagaaacacattgtcattatcggtgctggcgtgggtggcacggctacagctgctcgtttggcccgcgaaggcttcaaggtcactgtggtggagaaaaacgactttggtggcggccgctgctccttgatccatcaccagggccatcgctttgatcagggcccgtcgctctacctgatgcccaagtactttgaggacgcctttgccgatctggacgagcgcattcaagaccacctggagctgctgcgatgcgacaacaactacaaggtgcactttgacgacggtgagtcgatccagctgtcgtctgacttgacacgcatgaaggctgaattggaccgcgtggagggcccccttggttttggccgattcctggatttcatgaaagagacacacatccactacgaaagcggcaccctgattgcgctcaagaagaatttcgaatccatctgggacctgattcgcatcaagtacgctccagagatctttcgcttgcacctgtttggcaagatctacgaccgcgcttccaagtacttcaagaccaagaagatgcgcatggcattcacgtttcagaccatgtatatgggcatgtcgccctacgatgcgcctgctgtctacagcctgttgcagtacaccgagttcgctgaaggcatctggtatccccgtggcggcttcaacatggtggttcagaagctagaggcgattgcaaagcaaaagtacgatgccgagtttatctacaatgcgcctgttgccaagattaacaccgatgatgccaccaaacaagtgacaggtgtaaccttggaaaatggccacatcatcgatgccgatgcggttgtgtgtaacgcagatctggtctatgcttatcacaatctgttgcctccctgccgatggacgcaaaacacactggcttccaagaaattgacgtcttcttccatttccttctactggtccatgtccaccaaggtgcctcaattggacgtgcacaacatctttttggccgaggcttatcaggagagctttgacgaaatcttcaaggactttggcctgccttctgaagcctccttctacgtcaatgtgccctctcgcatcgatccttctgctgctcccgacggcaaggactctgtcattgtcttggtgcctattggtcatatgaagagcaagacgggcgatgcttccaccgagaactacccggccatggtggacaaggcacgcaagatggtgctggctgtgattgagcgtcgtctgggcatgtcgaatttcgccgacttgattgagcatgagcaagtcaatgatcccgctgtatggcagagcaagttcaatctgtggagaggctcaattctgggtttgtctcatgatgtgcttcaggtgctgtggttccgtcccagcacaaaggattctaccggtcgttatgataacctattctttgtgggtgcaagcacgcatcccggaactggtgttcccattgtccttgcaggaagcaagctcacctctgaccaagttgtcaagagctttggaaagacgcccaagccaagaaagatcgagatggagaacacgcaagcacctttggaggagcctgatgctgaatcgacattccctgtgtggttctggttgcgcgctgccttttgggtcatgtttatgttcttttacttcttccctcaatccaatggccaaacgcccgcatcttttatcaataatttgttacctgaagtattccgcgttcataactctaatgtcatttaaacgcgccactacaagtaggtactacaagtaggacagtatccccccaaaatgtaacataacacaaaataaatgtatctatacctaatcgacatggactgcaccgcattcgttatcttatcgtagtcctgcttactttgatccgtatcacattcgtgtatcgcgataagaagtaaacattgctgcaagtataaatcaggtctcaaatctcttcttgaagatagttttaagctcaccgtgaactgaacgtgaacactacgagtacgattttcaatattattaccgtcgatataacaaccatgcaatttctatatacatatatacattccctcattcgtctgtgctgtntggttcactgcagcaaatcaaacagcttttcgtactttctgtgctgcgaatgtttgttactatccgcgtgcattctttgcagctttngagatcccgtatagtccttccactagctagtaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagacacaaatgctgctcacctacatggaagtccacctctactacacgctgcctgtgctgggcgtcctgtcctggctgtcgcggccgtactacacagccaccgatgcgctcaaattcaaatttctgacactggttgccttcacgaccgcctccgcctgggacaactacattgtctaccacaaggcgtggtcctactgccccacctgcgtcaccgctgtcattggctacgtgcccttggaggagtacatgttcttcatcatcatgactctgttgaccgtggcattcaccaatctggtgatgcgctggcacctgcacagcttctttatcaggcctgaaacgcccgtcatgcagtccgtcctggtccgtcttgtccccataacagccttattaatcactgcatacaaggcttggcatttggcggtccctggaaagccactgttctacggatcatgcattttgtggtacgcctgtccggttttggccttattgtggtttggtgctggcgagtacatgatgcgtcgtccgctggcggtgctcgtctccattgcgctgcccacgctgtttctctgctgggtcgatgtcgtcgctattggcgccggcacatgggacatttcgctggccacaagcaccggcaagttcgtcgtgccccacctgcccgtggaggaattcatgttctttgcgctaattaataccgttttggtatttggtacgtgtgcgatcgatcgcacgatggcgatcctccacctgttcaaaaacaagagtccttatcagcgcccataccagcacagcaagtcgttcctccaccagatcctcgagatgacctgggccttctgtttacccgaccaagtgctgcattcagacacattccacgacctgtccgtcagctgggacatcctgcgcaaggcctccaagtccttttacacggcctctgctgtctttcccggcgacgtgcgccaagagctcggtgtgctatacgccttttgcagagccacggacgatctctgcgacaacgagcaggtccctgtgcagacgcgaaaggagcagctgatactgacacatcagttcgtcagcgatctgtttggccaaaagacaagcgcgccgactgccattgactgggacttttacaacgaccaactgcctgcctcgtgcatctctgccttcaagtcgttcacccgtttgcgccatgtgctggaagctggagccatcaaggaactgctcgacgggtacaagtgggatttggagcgtcgctccatcagggatcaggaggatctcagatattactcagcttgtgtcgccagcagtgttggtgaaatgtgcactcgcatcatactggcccacgccgacaagcccgcctcccgccagcaaacacagtggatcattcagcgtgcgcgtgaaatgggtctggtactccaatatacaaacattgcaagagacattgtcaccgacagcgaggaactgggcagatgctacctgcctcaggattggcttaccgagaaggaggtggcgctgattcaaggcggccttgcccgagaaattggcgaggagcgattgctctcactgtcgcatcgcctcatctaccaggcagacgagctcatggtggttgccaacaagggcatcgacaagctgcccagccattgtcaaggcggcgtgcgtgcggcctgcaacgtctatgcttccattggcaccaagctcaagtcttacaagcaccactatcccagcagagcacatgtcggcaattcgaaacgagtggaaattgctcttcttagcgtatacaacctttacaccgcgccaattgcgactagtagtaccacacattgcagacagggaaaaatgagaaatctaaataccatttaaacgcgtgcaattaacagatagtttgccggtgataattctcttaacctcccacactcctttgacataacgatttatgtaacgaaactgaaatttgaccagatattgttgtaaatagaaaatctggcttgtaggtggcaaactagtaacggccgccagtgtgctggaattgaatatttaccgttcgtataatgtatgctatacgaagttataccggtctcgtagtgttcacgttcagttcacggtgagcttaaaactatcttcaagaagagatttgagacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccataacttcgtataatgtatgctatacgaacggtacccgggcaattctgcagatatccatcacactggcggccgctcgagcatgcatctagatggcctccttggccgggtttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaccccccccccgcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcaggtgggccattctcatgaagaatatcttgaatttattgtcatattactagttggtgtggaagtccatatatcggtgatcaatatagtggttgacatgctggctagtcaacattgagccttttgatcatgcaaatatattacggtattttacaatcaaatatcaaacttaactattgactttataacttatttaggtggtaacattcttataaaaaagaaaaaaattactgcaaaacagtactagcttttaacttgtatcctaggttatctatgctgtctcaccatagagaatattacctatttcagaatgtatgtccatgattcgccgggtaaatacatataatacacaaatctggcttaataaagtctataatatatctcataaagaagtgctaaattggctagtgctatatatttttaagaaaatttcttttgactaagtccatatcgactttgtaaaagttcactttagcatacatatattacacgagccagaaattgtaacttttgcctaaaatcacaaattgcaaaatttaattgcttgcaaaaggtcacatgcttataatcaacttttttaaaaatttaaaatacttttttattttttatttttaaacataaatgaaataatttatttattgtttatgattaccgaaacataaaacctgctcaagaaaaagaaactgttttgtccttggaaaaaaagcactacctaggagcggccaaaatgccgaggctttcatagcttaaactctttacagaaaataggcattatagatcagttcgagttttcttattcttccttccggttttatcgtcacagttttacagtaaataagtatcacctcttagagttaactatgagataagcaagtatcatctcatttcatttacctgaagtcgagtaaacagaaaatccaattgttgatgaacctcaatgacttagaactatctatcggcagatcatataaagaggatttaggtacctagaggactgtacctggagtatatatatatatatatatatattatctcaactatagtccatagaggtttctttcttgaggccttaaactgctaaagaatgatattggtggaatgcaagcaccaatctctcttctttcgtaactgttcatatacttcaaaccaagaatgtaacgggcattgacccatccaaaaccttcagtagctgcccctttaaagtcagcaccttgattaccgtattctgcttcaacacgatgaggatctgttcctcttgtgacatcatatttttcaaccacaataccattataatcgacaaaagcctttgtcatcatgaaaagccatctataagctagcctattcgttacagttaaataaccataagaacggaggccttcccaagcaagaatttgatggggtgcccaaccaaatggatagtcccattgtctaattggtctcgaaatagaaattgggcctcgagaacgctccgtacatgcagctaaacctccaagcatctctaacttgggtagtgctttctccaccattttctgtgcttgctccttcgtggcaagtccagcccataatgcccagaatgtagttgcggattcgtatgacgttctgtgcttgatttttgtgttgtagtcaaagaaaaaccccgactcgtcatcccacatatatttggtaattgatgaggcaacgctaattatcaacatatagattgttatctatctgcatgaacacgaaatctttacttgacgacttgaggctgatggtgtttatgcaaagaaaccactgtgtttaatatgtgtcactgtttgatattactgtcagcgtagaagataatagtaaaagcggttaataagtgtatttgagataagtgtgataaagtttttacagcgaaaagacgataaatacaagaaaatgattacgaggatacggagagaggtatgtacatgtgtatttatatactaagctgccggcggttgtttgcaagaccgagaaaaggctagcaagaatcgggtcattgtagcgtatgcgcctgtgaacattctcttcaacaagtttgattccattgcggtgaaatggtaaaagtcaaccccctgcgatgtatattttcctgtacaatcaatcaaaaagccaaatgatttagcattatctttacatcttgttattttacagattttatgtttagatcttttatgcttgcttttcaaaaggcctgcaggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttc

SEQ ID NO: 5 - DNA, 13246 bp, synthetic construct
  source 1..13246: mol_type = other DNA
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgacgagtctgatgagtccgtgaggacgaaacgagtaagctcgtcactcgtctacgcatgcctgggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgttggaccccgtcttcaattacacttcccaactgggaacacccctctttatcgacccattttaggtaatttaccctagcccattgtctccataaggaatattaccctaacccacagtccagggtgcccaggtccttctttggccaaattttaacttcggtcctatggcacagcggtagcgcgtgagattgcaaatcttaaggtcccgagttcgaatctcggtgggacctagttgaaaaatacctctaatgcgccgatggtttagtggtaaaatccatcgttgccatcgatgggcccccggttcgattccgggtcggcgcaggttgacgtcgaacgtcttgtcttccgaagttttagagctagaaatagcaagttaaaataaggctagtccgttatcaacttgaaaaagtggcaccgagtcggtgcttttttttttcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

SEQ ID NO: 6 - DNA, 2611 bp, synthetic construct
  source 1..2611: mol_type = other DNA
aggccaagagactgtgaggcccatgtccacttggtcacgggttgacaaaaagaaacgcctccttccctgtgtttgtccacgatggccagaataccacgcagtgctagaatatttctgtatttttgaaatatcaattttgaccacgtccgactccaaatagcaccatataaccaacccctggtttagacccacacacactacttaccacctgcagcacatcacgtcgacatccccgcgatcaatcgcatcagaaaccctagccctatttcgggaggtcagctctcatgcagtcaccagctcccctaaccctatttctcgttctcctaacagcgctatcagagattcgctatcagaaactctaaatttattaccggattggggaccgattgggagttggagcgccctaaccctattgttagcactaccctagccctatttcaaactcccaacaggcaaaatgcaccacaattttgacacaaagtcccaatactggaaaatcttgacaaccaagggggccaaacatgcaccatgaattaggcgcaaacacgggtatttcgacacaacacagcagagcatactctaaattactcccggccaaacaacacagcacgctccgcagcaaatctatttaaacactcccacgcccccccagaaatccttcttcacacaacacaacaactcaaccactccaacaaacaaatacaatgacccagtccgtcaaggtcgttgagaagcacgtccccattgtcattgagaagccctccgagaaagaggaagatacctcttccgaggactccatcgagctcaccgtcggcaagcagcccaagcccgtcaccgagactcgatctctcgacgatctcgaggccatcatgaaggctggtaagaccaagctgctcgaggaccacgaggttgtcaagctctctctcgagggcaagctgcccctctacgctctcgagaagcagctcggtgacaacacccgagccgtcggtatccgacgatccatcatctcccagcagtccaacaccaagaccctcgagacttccaagctcccctacctgcactacgactacgaccgagttttcggtgcctgctgcgagaacgtcatcggttacatgcctctgcccgtcggtgttgccggccccatgaacattgacggcaagaactaccacatccccatggccaccaccgagggctgtctggttgcttccaccatgcgaggctgcaaggccatcaacgctggtggtggtgtcaccaccgttctcacccaggacggtatgacccgaggtccttgtgtctccttcccctctctcaagcgagctggtgctgccaagatctggctcgactccgaggaaggcctcaagtccatgcgaaaggccttcaactccacttctcgatttgcccgactccagtctctgcactccactctcgccggtaacctcctcttcatccgattccgaaccaccaccggtgacgccatgggtatgaacatgatctccaagggtgtcgagcactctctggccgtcatggtcaaggagtacggtttccccgacatggacattgtctctgtttccggcaactactgcaccgacaagaagcccgctgccatcaactggatcgagggccgaggcaagtctgttgttgctgaggccaccatccccgcccacattgtcaagtccgttctcaagtccgaggtcgatgctctggtcgagctcaacatctccaagaacctcattggctctgccatggccggctccgtcggtggtttcaacgcccacgccgccaacctggtcaccgccatctacctcgccaccggccaggaccccgcccagaacgtcgagtcctccaactgtatcaccctcatgtccaacgtcgacggtaacctgctcatctctgtctccatgccctccattgaggtcggtaccattggtggcggtaccatcctcgagccccagggtgccatgctcgagatgctcggtgtccgaggcccccacattgagactcccggtgccaacgcccagcagctcgcccgaatcattgcctccggtgttctggctgctgagctgtctctgtgctctgctctggccgccggccacctcgtccagtcccacatgacccacaaccgatcccaggctcccactcccgccaagcagtcccaggccgatctccagcgactccagaacggctccaacatctgtatccgtagctaaacagtttgtttagcaaaatatatttaacgagtttgatagaggcgctggactacataattactgaatcacgcgtacatgtttcagctcaaattgtatcacggtttctttgtagcaatggagggggagagttgacaaggcattagagaagagagcgagaggagaagacaagtggatagacgactgcaatcatatgatctgcacaaactgcgatgttttcctgtcagatcatgttcttttgctcatagttaagctatcgtgactttacggatccgccgagcatcttagtagcgaggtttgttgcctcgtgtctccaggcctcctcttgtgtgtgattccgaagcatagtgtacgattggtaaattggtgatgaggggcctaccgtgtggcaatccagaggctcgagg

SEQ ID NO: 7 - DNA, 10383 bp, synthetic construct
  source 1..10383: mol_type = other DNA
tatgcaaaagcacgaagcaagtgtctcccacgtaggccgttggacgccgctcctgtacgcactttgtcccacaaattcccgattccgcaatttgttcgccgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgtccaagaagcacattgtcatcattggtgctggtgtcggtggtactgccaccgccgcccgactcgcccgagagggtttcaaggtcaccgtcgttgagaagaacgacttcggtggtggtcgatgctctctcatccaccaccagggccaccgattcgaccagggcccctctctctacctcatgcccaagtacttcgaggacgcctttgccgacctcgacgagcgaatccaggaccacctcgagctgctccgatgtgacaacaactacaaggtccactttgacgacggcgagtccatccagctctcttccgatctgacccgaatgaaggccgagctcgaccgagtcgagggtcctctcggctttggccgattcctcgactttatgaaggagactcacatccactacgagtccggtaccctcattgctctcaagaagaactttgagtccatctgggatctcatccgaatcaagtacgcccccgagattttccgactccacctcttcggcaagatctacgaccgagcctccaagtatttcaagaccaagaagatgcgaatggccttcaccttccagactatgtacatgggcatgtccccctacgacgcccccgctgtctactctctgctccagtacaccgagtttgccgagggtatctggtacccccgaggcggcttcaacatggttgtccagaagctcgaggccattgccaagcagaagtacgatgctgagttcatctacaacgctcccgttgccaagatcaacaccgacgacgccaccaagcaggtcaccggtgtcactctcgagaacggccacatcatcgacgccgacgccgttgtctgcaacgccgatctggtctacgcctaccacaacctgctgcctccctgccgatggacccagaacaccctcgcctctaagaagctgacctcctcctccatctccttctactggtccatgtccaccaaggtcccccagctcgacgtccacaacatcttcctcgccgaggcctaccaggagtctttcgacgagatcttcaaggactttggtctgccctccgaggcttctttctacgtcaacgttccctctcgaatcgacccctctgctgctcccgatggtaaggactccgtcattgttctcgtccccatcggccacatgaagtccaagaccggtgatgcttccaccgagaactaccccgccatggtcgacaaggctcgaaagatggttcttgctgtcattgagcgacgactcggtatgtccaactttgccgatctcattgagcacgagcaggtcaacgaccccgccgtctggcagtccaagttcaacctgtggcgaggctccatcctcggtctgtcccacgacgttctccaggttctgtggttccgaccctctaccaaggactccaccggccgatacgacaacctcttcttcgtcggtgcctccacccaccccggtactggtgtccccattgttctggccggttccaagctcacctccgaccaggttgtcaagtctttcggtaagacccccaagccccgaaagatcgagatggagaacacccaggctcctctggaggagcccgacgccgagtctactttccccgtctggttctggctccgagctgccttctgggtcatgttcatgttcttctacttcttcccccagtccaacggccagacccccgcctccttcatcaacaacctcctccccgaggtcttccgagtccacaactccaacgtaatttaaaggatataaaagtgcatgcacgagagtgtaataaatttataatcgaacgactgacaggccagtgcgatccacgagtctggccggtgacacgagcaccatgtagataacacgacgacaaatgacggtgacaagagcatgggcggagtttgactgatggaggatgtagatctggatttccgctgggatgtcagtatgaaatgctgaaattctaccgagtgtcctcgttatcgtgtggtatctgctatatctgcaaaggccatgctggtgtgttgtggttatcgtgtgtggagtgtagatgttcctcgaacaggatatttcgatcttggatacgtactcgcttgtgtctggtttcgtgtgccgggttggcggcgcatttgtgtcccaaaaaacagccccaattgccccaattgaccccaaattgacccagtagcgggcccaaccccggcgagagcccccttctccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatgctcctgacctacatggaggtccacctctactacaccctgcccgttctcggtgttctgtcttggctctcccgaccttactacaccgccaccgacgctctcaagttcaagttcctgaccctcgttgcctttaccactgcctccgcctgggacaactacatcgtctaccacaaggcctggtcctactgccccacctgtgtcaccgccgtcattggttacgttcctctcgaggagtacatgttctttatcatcatgaccctgctcaccgtcgccttcaccaacctcgtcatgcgatggcacctccactccttcttcatccgacccgagactcccgtcatgcagtctgttctcgtccgacttgtccccatcactgctctgctcatcaccgcctacaaggcttggcaccttgctgtccccggcaagcccctcttctacggctcttgtatcctgtggtacgcttgccccgtcctcgctctcctctggtttggcgccggcgagtacatgatgcgacgacccctggccgttctcgtctccattgccctgcccactctcttcctgtgctgggtcgatgttgtcgccattggtgccggtacctgggacatctctctggccacttccactggtaagttcgttgtcccccacctccccgtcgaggagttcatgttcttcgccctcatcaacaccgtcctcgtctttggtacctgtgccatcgaccgaaccatggccatcctccacctcttcaagaacaagtctccttaccagcgaccctaccagcactccaagtccttcctgcaccagattctcgagatgacctgggccttctgtctgcccgaccaggttctgcactctgacaccttccacgacctctccgtctcttgggatatcctccgaaaggcctctaagtccttctacaccgcttccgctgtcttccccggtgacgtccgacaggagcttggtgttctctacgccttctgccgagccaccgatgacctctgtgacaacgagcaggtccccgtccagacccgaaaggagcagctcatcctcacccaccagtttgtctccgacctctttggccagaagacctccgcccccaccgccattgactgggacttctacaacgaccagctccccgcttcctgcatctccgccttcaagtctttcacccgactccgacacgttctcgaggctggtgccatcaaggagctgctcgacggttacaagtgggatctcgagcgacgatccatccgagatcaagaggacctccgatactactccgcctgtgttgcctcttccgtcggtgagatgtgcacccgaatcatcctcgcccacgctgacaagcccgcctcccgacagcagactcagtggatcatccagcgagcccgagagatgggcctcgttctccagtacaccaacattgcccgagacattgtcactgactccgaggagctcggccgatgctacctcccccaggactggctcaccgagaaagaggttgctctcatccagggcggtcttgctcgagagattggtgaggagcgactcctctctctgtcccaccgactcatctaccaggctgacgagctcatggtcgtcgccaacaagggtatcgacaagctcccctcccactgccagggcggcgtccgagctgcctgcaacgtctacgcctccatcggtaccaagctcaagtcctacaagcaccactacccctctcgagcccacgtcggtaactccaagcgagtcgagattgctctgctgtccgtctacaacctgtacaccgcccccattgccacctcctccaccacccactgccgacagggcaagatgcgaaacctcaacacgatctaaactatttacagcatgtgtaatgaggaatataacgttgattgaattgtttgtgaaaaatgtagaaaatttcagtgaagttgtgttttctatatagtaagcacttttggtacaagtatctgcacatccctgcatgttacaagcctgatcatgcagggcaatattctgactataaatatacctcgatattttagcaagctatagttgcactaactaaccaactagtatcttctaaacctctctgcaccttctttcgcttgcactgttcccagactccaagatttgtatacgtgcttgacaaatagctcaagggcgtggagcacagggtttaggaggttttaatgggcgagaaggcgcgtagatgtagtcttcctcggtcccatcggtaatcacgtgtgtgccgatttgcaagacgaaaagccacgagaataaaccgggagaggggatggaagtccccgaacagcaaccagcccttgccctcgtggacataacctttcacttgccagaactctaagcgtcaccacggtatacaagcgcacgtagaagattgtggaagtcgtgttggagactgttgatttgggcggtggaggggggtatttgagagcaagtttgagatttgtgccattgagggggaggttattgtggccatgcagtcggatttgccgtcacgggaccgcaacatgcttttcattgcagtccttcaactatccatctcacctcccccaatggcttttaactttcgaatgacgaaagcacccccctttgtacagatgactatttgggaccaatccaatagcgcaattgggtttgcatcatgtataaaaggagcaatcccccactagttataaagtcacaagtatctcagtatacccgtctaaccacacatttatcacaatgaccgatgtccgattccgaatcatcggtaccggtgcctacgttcccgagcgaattgtctccaacgacgaggttggtgctcccgctggtgttgacgacgactggatcacccgaaagaccggtatccgacagcgacgatgggctgctgacgaccaggccacctccgatctcgccaccgctgccggccgagctgctctcaaggctgctggtatcacccccgagcagctgaccgtcattgccgttgccacctctacccccgaccgaccccagcctcccactgccgcctacgtccagcaccacctcggtgccaccggcaccgccgccttcgatgtcaacgccgtctgctccggtaccgtctttgccctctcttccgtcgccggtactctggtctaccgaggcggctacgccctcgtcattggtgctgacctctactcccgaatcctcaaccccgccgaccgaaagactgttgttctcttcggtgacggtgccggcgccatggttctcggccccacctccactggtaccggccccattgtccgacgagttgctctgcacaccttcggtggtctgaccgatctcatccgagtccccgccggtggctctcgacagcctctcgacaccgacggtctggacgccggtctgcagtacttcgccatggacggccgagaggtccgacgattcgtcaccgagcacctgccccagctcatcaagggcttcctccacgaggctggtgtcgacgccgctgacatctcccactttgtcccccaccaggccaacggtgtcatgctcgacgaggtctttggcgagctccacctcccccgagccaccatgcaccgaaccgtcgagacttacggcaacaccggtgctgcctccatccccatcaccatggacgccgccgtccgagccggttccttccgacccggagagctcgttctgctcgccggcttcggcggtggtatggccgcctctttcgccctcattgagtggtaaaccaacagtagcatgacgagtatatagtgttttatgaatttaatgtgtatacaagggtgtgtgtagttgagtaatgagaaaccccaatcatcatctgcctaaagctaacgaggattgtaacgacaccgtatgtactcaccacaggtacaaagtacaactacgagcatgtacagtataccaagatcggtgatgacttgaatttgtacagctcaaaccccccagatacggtataatacacagttctgtacaagccgtcacaattgccaaagttgcaaaaatgattgtataaaaatatatgctcctcccaatacaaggcagaactttcctccaccgtatccgctcgcaatttctttcgtgcgggaagcgaaccggcaatattccacaatgtgctggcatttacttgtgctggcaaaagaggcacaaagaatacttgtagtcggagccactcactgtcccacaaatagctccccgctgtcaatctctcctgcaccgcctgctcacatggatgctaagccgcactaggtcgcatatatggctctgcactaaaaattaggggtcaaccacagtgcggtatttttagattcgcaccaagcagcgagtaagcaaaaatacgcctaccggggtccgatattattcaggaggtgccattagaggagggcagatgagagtcggatatcggagatattaccgaggctataattaccccatccacgcctttcacccctcccactctctccctcaccgcacaccaacccaccactttcaaaatataccgcaacattgacataatctccggtacagtggttagcaccgagaggaccccaaaaagcttgggggagatagaggtaggcttttttttgtcagtcaaatcgtatatgccaatacacacacacacacacacacacacacacagtttcgtacataacagtatattggaagggagtgtgcttggcaaagacaggagaagacggtgctgttagagggcaatccagacgggctagagcgctgtaactttcggatcgatttcaattcctctagaataccaaataccagtggttaagcggctcatttaccagtcctaataccccctccaccagccaccttcccctattcctcggcagtgcttttttacctttgagatgtggccttgtctccgttacttcccaaccgtgagtgctgtgtggtgtgctggacagtgcgacataactaaccctaacccagacgagccagcgcaccccaattttgtgtttgccaactcctacttttctcctctcctccatcggtatttcatcgacaaatctctttgctaccaacaaccacacaaattaaaaatggactacatcatctccgctcccggcaaggtcatcctcttcggtgagcacgccgccgtctttggcaagcccgccattgctgctgccatcgatctgcgaacctacctcctcgtcgaaaccaccacctccgacacccccaccgtcactctcgagttccccgatatccaccttaacttcaaggttcaggtcgacaagctcgcctctctcactgcccagaccaaggccgaccacctcaactggtccacccccaagaccctcgacaagcacatcttcgactctctctcttctctcgctctgctggaggagcccggtctgaccaaggtccagcaggctgctgttgtttctttcctgtacctctacatccacctctgtcctccctccgtctgcgaggactcctccaactgggttgtccgatccaccctccccattggtgctggcctcggttcttctgcctccatctgtgtctgcctcgccgccggcctcctcgttctcaacggccagctctccatcgaccaggctcgagacttcaagtctctgaccgagaagcagctgtctctggtcgacgactggtccttcgtcggtgagatgtgcatccacggcaacccctccggtatcgacaacgccgttgccacccagggtggtgctctgctgttccagcgacccaacaaccgagttcctctcgttgatatccccgagatgaagctgctgctcaccaacaccaagcacccccgatccactgccgatctggtcggtggtgtcggtgttctcaccaaggagtttggctccatcatggaccccatcatgacctccgtcggcgagatctccaaccaggccatggagatcatctcccgaggcaagaagatggttgaccagtccaacctcgagattgagcagggtatcctcccccagcccacttccgaggacgcctgcaacgtcatggaggacggtgccaccctccagaagctccgagacattggctccgagatgcagcacctcgtccgaatcaaccacggtctgctcattgccatgggtgtctcccaccccaagctcgagatcatccgaaccgcctccattgtccacaacctcggtgaaaccaagctcaccggtgctggtggtggtggctgtgccatcaccctcgtcacctccaaggacaagaccgccacccagctcgaggagaacgtcattgccttcactgaggagatggccacccacggcttcgaggtccacgagactaccattggcgcccgaggtgtcggtatgtgcattgaccacccctctctcaagactgtcgaggccttcaagaaggtcgagcgagccgatctcaagaacattggcccctggacccactaaacgtaaaagcggtatgtattaattgagagttttataagacagtgtttgcgcgagctgacgagtgtttgccgagtgcatatcacaacgagaacctgagagccacgcaatgtgtggttgtagtgtggatatgcccatgagacaatggtagtgtggagaatacccatgagacgatggtaatgtaaagaatggcccacgagatgcaattgaggcccaattgactaattgatattgtagaccgagtcaatgggtcagtgcagaggagcggcgtttgctcaattgactcgggaacggacatgtgcacctcggtgttgcacacgaatacgacacttcccaatacactttgcctctccaaggggtaacatgatgtaatggcgcgttccacaagacccaagacatttgttact